(19) United States
(12) Patent Application Publication
(10) Pub. No.: US 2020/0277449 A1
(43) Pub. Date: Sep. 3, 2020

(54) INJECTABLE HYBRID ALGINATE HYDROGELS AND USES THEREOF

(71) Applicants: Université de Strasbourg, Strasbourg (FR); Centre National de la Recherche Scientifique, Paris (FR); IHU Strasbourg - Institut Hospitalo-Universitaire de Strasbourg, Strasbourg (FR)

(72) Inventors: Luisa DE COLA, Strasbourg (FR); Silvana PERRETTA, Strasbourg (FR); Giuseppe ALONCI, Strasbourg (FR); Pietro RIVA, Cesano Maderno (IT); Etienne PIANTANIDA, Strasbourg (FR); Ludovica GUERRIERO, Napoli (IT)

(21) Appl. No.: 16/647,225

(22) PCT Filed: Sep. 17, 2018
(86) PCT No.: PCT/EP2018/075097

§ 371 Date: Mar. 13, 2020

(30) Foreign Application Priority Data
Sep. 15, 2017 (EP) 17306195.3
Dec. 1, 2017 (EP) 17306692.9
Dec. 1, 2017 (EP) 17306693.7
Jan. 17, 2018 (EP) 18152172.5
Jan. 17, 2018 (EP) 18152173.3

(51) Int. Cl. C08J 3/075; A61L 27/26; A61L 27/52; A61K 8/04; A61K 8/88; A61K 8/73; A61Q 19/00; A61K 47/36; A61K 9/06; A61K 9/51; C08K 3/36

(52) U.S. Cl. CPC C08J 3/075; A61L 27/26; A61L 27/52; A61K 8/042; A61K 8/88; A61K 8/733; B82Y 5/00; A61K 47/36; A61K 9/06; A61K 9/5161; C08K 3/36; C08K 2201/011; A61Q 19/00

(57) ABSTRACT

The invention relates to a hybrid hydrogel, in particular degradable or non-degradable, comprising a first hydrogel polymer of formula (I) in association with an alginate hydrogel polymer, and optionally organosilica particles in particular degradable or non-degradable nanoparticles, or porous silicon particles; pharmaceutical, veterinary and/or cosmetic compositions thereof; and uses thereof as a medicament. The invention notably relates to the use of such hybrid hydrogel in the treatment of fistulas and physiological leaks/leakages, notably in the gastrointestinal tract. The present invention finds applications in the therapeutic and diagnostic medical technical fields and also in cosmetic and veterinary technical fields.

### PRIORITY

This PCT Application claims priority to European Provisional Patent Application n^{o} EP 17306195.3 filed on 15 Sep. 2017; European Provisional Patent Application n^{o} EP 17306692.9 filed on 1 Dec. 2017; European Provisional Patent Application n^{o} EP 17306693.7 filed on 1 Dec. 2017; European Patent Application n^{o} EP 18152172.5 filed on 17 Jan. 2018; and European Patent Application n^{o} EP 18152173.3 filed on 17 Jan. 2018; the entire contents of each of which are hereby incorporated by reference.

### FIELD

The invention relates to a hybrid hydrogel, in particular degradable or non-degradable, comprising a first hydrogel polymer of formula (I) in association with an alginate hydrogel polymer, and optionally organosilica particles in particular degradable or non-degradable nanoparticles, or porous silicon particles; pharmaceutical, veterinary and/or cosmetic compositions thereof; and uses thereof as a medicament. The invention notably relates to the use of such hybrid hydrogel in the treatment of fistulas and physiological leaks/leakages, notably in the gastrointestinal tract.

The present invention finds applications in the therapeutic and diagnostic medical technical fields and also in cosmetic and veterinary technical fields.

### BACKGROUND

Biocompatible soft materials, and in particular hydrogels and liquids that can form interlayers between tissues, have been recently used in surgery to facilitate resection of tumors.

They find applications as submucosal fluid cushions (SFC), to avoid perforation and thus to facilitate endoscopic submucosal dissection (ESD). ESD is a clinical procedure applied for early stage neoplastic lesions in the gastrointestinal tract that allows en bloc resection of large lesions. During surgery it is necessary to lift the mucosa and to prevent the occurrence of damages to deeper tissues.

The injection of materials, which can gel in situ forming a protective layer between the part to be removed and the healthy tissue, has been proposed as a way to avoid perforation and thus to facilitate ESD.

Even though several injection solutions have been proposed and tested, normal saline solution (NS) is the most commonly used in the clinic because of its low cost and ease of use. However, it is hampered by low mucosal elevations, making the procedure difficult and often resulting in electrocautery damage of the muscularis (i.e. the thin layer of muscle of the GI tract, which is located underneath the submucosa). In addition, its rapid absorption in the surrounding tissues requires repeated injections for extensive resection.^{[8]}

Various substances, including glycerol,^{[10]} hydroxypropyl methylcellulose^{[11]} and hyaluronic acid^{[12]} have been exploited to achieve sustained mucosal elevation and avoid injuries to the muscularis propria. Although hyaluronic acid solution is one of the best options,^{[12]} it has been shown to induce a serious side effect which corresponds to a stimulation of the growth of residual tumors proliferation in animal models.^{[13]} Moreover, a large amount of hyaluronic acid is necessary to create a SFC and its use is associated with high costs (US $550.58/g) and a general lack of availability.^{[7]}

In the last years, injectable hydrogels have brought a shift in the search for the optimal SFC material towards the development of solutions that rely on in situ gel formation. For example, a photo-crosslinked chitosan hydrogel has been recently reported as a submucosal injection agent: mucosal elevation was created after the injection of the chitosan viscous solution, which was crosslinked in situ via UV irradiation, resulting in an insoluble hydrogel.

However, the use of UV light for the photoinitiated radical polymerization may be difficult in hard-to-reach areas and resulted somehow inconvenient as performed by the authors: was irradiated with UV light for a total of 5 min (30 s each at 10 different places by using an UV light-fiber through the endoscopic accessory channel and UV lamp system). Moreover, the authors mentioned that UV irradiation may be associated with inflammation of the residual tissue.

Thermoresponsive polymers, or thermogels, have been investigated as well for ESD applications, such as the recently proposed water solution of a PEG/PLGA-based temperature-sensitive polymer. However, many of these materials have been shown to clog inside long delivery tools at normal body temperature.

There is therefore a real need to find a compound which allows more efficient treatment and/or effective treatment and/or a compound which is not rapidly absorbed after injection and/or a compound that would reduce the number of injections.

There is also a real need to find a product/compound that could be clinically applicable, for example be biocompatible, easily injectable, able to provide a prolonged and thick SFC to allow the ESD procedure safely, cost-effective.

Biocompatible soft materials, and in particular hydrogels have been also proposed as dressing for example for topical wound. For example, hydrogels are particularly useful on superficial and deep chronic wounds, ulcers, leg ulcers, restorative and reconstructive surgery, sluggish wounds, dermabrasion, severe sunburn, superficial and deep burns of the second degree. Such dressings are commercially available, for example, Askina Gel sold by B Braun, Duoderm Hydrogel sold by Convatec, Hydrosorb sold by Hartmann, IntraSite Gel marketed by Smith & Nephew, Normgel sold by Molnlycke, Purilon sold by Coloplas and Urgo hydrogel sold by Urgo.

However, known hydrogels have limited spectra of uses and are particularly designed to fit to specific wound and/or to be used in particular environments. In addition, known hydrogel are most of the time roughly applied onto the surface of the wound and cannot be injected at the wound and/or lesion site.

In addition, when the lesion and/or the wound is located between tissues and/or at the interface of tissues, for example in the gastrointestinal tract and/or at chirurgical site within the body of a mammal, the known hydrogels can, most of the time, not be used due to their rheological and/or biocompatible properties. In particular, most of the known hydrogels used as wound dressing are not biocompatible and/or biodegradable in-situ.

In addition, the known hydrogels are reticulated previously to their use and thus cannot be injected, for example with a needle, due to their viscosity.

There is thus a real need to find a product/compound that could be used as fillers and/or materials that could be injected/applied onto/into a wound. There is also a need to find product/compound, that could be an adaptive to fit to any wound whatever its form and/or size and/or localization.

There is also a need to find a biocompatible and/or biodegradable product that could avoid/reduce after injection side effect such as inflammation and be naturally resorbed and/or degraded after injection.

There is also a real need to find a product/compound that could be controlled and be tunably biodegradable, and/or could possibility release active components and/or molecules, for example to prevent bacterial infection and/or enhance healing at the lesion site.

### Definitions

To facilitate an understanding of the present invention, a number of terms and phrases are defined below:

As used herein other than the claims, the terms “a,” “an,” “the,” and/or “said” means one or more. As used herein in the claim(s), when used in conjunction with the words “comprise,” “comprises” and/or “comprising,” the words “a,” “an,” “the,” and/or “said” may mean one or more than one. As used herein and in the claims, the terms “having,” “has,” “is,” “have,” “including,” “includes,” and/or “include” has the same meaning as “comprising,” “comprises,” and “comprise.” As used herein and in the claims “another” may mean at least a second or more. As used herein and in the claims, “about” refers to any inherent measurement error or a rounding of digits for a value (e.g., a measured value, calculated value such as a ratio), and thus the term “about” may be used with any value and/or range.

The phrase “a combination thereof” “a mixture thereof” and such like following a listing, the use of “and/or” as part of a listing, a listing in a table, the use of “etc” as part of a listing, the phrase “such as,” and/or a listing within brackets with “e.g.,” or i.e., refers to any combination (e.g., any sub-set) of a set of listed components, and combinations and/or mixtures of related species and/or embodiments described herein though not directly placed in such a listing are also contemplated. Such related and/or like genera(s), sub-genera(s), specie(s), and/or embodiment(s) described herein are contemplated both in the form of an individual component that may be claimed, as well as a mixture and/or a combination that may be described in the claims as “at least one selected from,” “a mixture thereof” and/or “a combination thereof.”

In general, the term “substituted” whether preceded by the term “optionally” or not, and substituents contained in formulae of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds.

As used herein, the term “alkyl”, refers to straight and branched alkyl groups. An analogous convention applies to other generic terms such as “alkenyl”, “alkynyl” and the like. In certain embodiments, as used herein, “lower alkyl” is used to indicate those alkyl groups (substituted, unsubstituted, branched or unbranched) having about 1-6 carbon atoms. Illustrative alkyl groups include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “C₁₋ₓalkylenyl”, as used herein, refers to a linear or branched saturated divalent radical consisting solely of carbon and hydrogen atoms, having from one to x carbon atoms, having a free valence “-” at both ends of the radical. Likewise, the term “C₁₋ₓheteroalkylenyl”, as used herein, refers to a linear or branched saturated divalent C₁₋ₓalkylenyl radical as defined above, comprising at least one heteroatom such as O, N, or S, and having a free valence “-” at both ends of the radical. When the C₁₋ₓalkylenyl or C₁₋ₓheteroalkylenyl is optionally substituted, at least one of the H atoms may be replaced by a substituent such as halogen or —OR where R may represent C1-6alkyl.

The term “ethenylenyl”, as used herein, refers to the divalent radical —CH═CH—. When the ethenylenyl is optionally substituted, one or both the H atoms may be replaced by a substituent such as halogen or —OR where R may represent C1-6alkyl.

In general, the term “aromatic moiety” or “aryl”, as used herein, refers to stable substituted or unsubstituted unsaturated mono- or polycyclic hydrocarbon moieties having preferably 3-14 carbon atoms, comprising at least one ring satisfying the Hackle rule for aromaticity. Examples of aromatic moieties include, but are not limited to, phenyl, indanyl, indenyl, naphthyl, phenanthryl and anthracyl.

The term “halogen” as used herein refers to an atom selected from fluorine, chlorine, bromine and iodine.

As used herein, the term “independently” refers to the fact that the substituents, atoms or moieties to which these terms refer, are selected from the list of variables independently from each other (i.e., they may be identical or the same).

As used herein, the term “template” or “supramolecular template” refers to a self-aggregation of ionic or non-ionic molecules or polymers that have a structure directing function for another molecule or polymer.

As used herein, the term “and/or” means any one of the items, any combination of the items, or all of the items with which this term is associated.

As used herein, the term “about” refers to a variation of +5-10% of the value specified. For example, “about 50” percent can in some embodiments carry a variation from 45 to 55 percent. For integer ranges, the term “about” can include one or two integers greater than and/or less than a recited integer. Unless indicated otherwise herein, the term “about” is intended to include values, e.g., weight percents, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible subranges and combinations of subranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to,” “at least,” “greater than,” “less than,” “more than,” “or more,” and the like, include the number recited and such terms refer to ranges that can be subsequently broken down into subranges as discussed above. In the same manner, all ratios recited herein also include all subratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

An “effective amount” refers to an amount effective to treat a disease, disorder, and/or condition, or to bring about a recited effect. For example, an amount effective can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” extend to prophylaxis and include prevent, prevention, preventing, lowering, stopping or reversing the progression or severity of the condition or symptoms being treated. As such, the term “treatment” includes medical, therapeutic, and/or prophylactic administration, as appropriate.

The term “responsively disintegrable”, when referring to organosilica matrix/material used as part of the hydrogels according to the invention, refers to the property of the organosilica material that undergoes degradation (i.e., breakdown of the structural integrity of the organosilica material) triggered by a particular stimulus. The stimulus can be, for example, a change in pH (either an increase or decrease), a change in redox potential, the presence of reduction or oxidation agent, the presence of UV, visible, infrared or near infrared light, ultrasounds, electromagnetic radiation, an enzymatic cleavage, a change in temperature, etc. The organosilica material may be porous (preferably mesoporous), and may be in the form of an organosilica nanoparticle or core-shell nanocapsule, as further described herein.

The term “responsively cleavable”, when referring to a chemical bond, polymer fragment or linking group, refers to a covalent bond, polymer fragment or linking group that is cleaved upon application of one of the aforementioned particular stimuli. Generally speaking, the presence of a responsively cleavable bond, polymer fragment or linker moiety within the framework of a hydrogel according to the invention confers to the hydrogel properties of degradation (it becomes degradable upon application of said stimulus). Likewise, the presence of a responsively cleavable bond, polymer fragment or linker moiety within the structure of an organosilica material that may be used in mixture with and/or that may be covalently conjugated to the framework of a hydrogel polymer or non-covalently embedded (mixed in or dispersed) within the hydrogel network according to the invention, confers to the organosilica matrix/material its disintegrable properties (the property of structurally breaking down upon application of a specific signal/stimulus, akin to “self-destructive” behavior). When the organosilica material is covalently conjugated to the hydrogel framework, for example as part of a molecular crosslinker of structure:

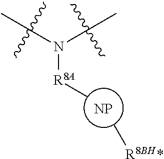

as further detailed and defined herein, the presence of a responsively cleavable bond, polymer fragment or linker moiety within the organosilica matrix confers in turn responsively degradable properties to the hydrogel (i.e., the hydrogel becomes degradable upon application of one of the aforementioned particular stimuli). As mentioned above, the organosilica material may be porous (preferably mesoporous), and may be in the form of an organosilica nanoparticle or core-shell nanocapsule, as further described herein.

Conversely, the term “stable covalent bond” refers to a covalent bond that is not cleaved in the environment to which it is exposed and/or upon application of one of the aforementioned particular signals. In that sense, the term “stable covalent bond” may be used interchangeably with “non-responsively cleavable covalent bond”.

As used herein, the term “hydrogel” refers to polymers comprising a solid polymer lattice and an interstitial aqueous phase.

As used herein, the term “degradable hydrogel” refers to hydrogels comprising at least one crosslinker within its structure, which can be cleaved upon application of a suitable signal/stimulus, or by biodegradation of the linker, resulting in the breakdown of the hydrogel crosslinked structure. For example, the hydrogel may comprise a redox-responsive crosslinker, such as cystamine crosslinker, which can be cleaved in response to a change in the redox potential of the environment. For example, a cystamine crosslinker may cleave in response to a variation in glutathione concentration in the surrounding environment. In yet another example, the hydrogel may comprise a pH-responsive crosslinker, such as an imine-bond containing crosslinker, which can be cleaved in response to a change in pH of the environment. The hydrogel may be said to be biodegradable when the environment is a physiological environment, and/or when the hydrogel contains at least one crosslinker which can undergo cleavage by biological means (bacteria, enzymes, etc.). Examples of degradable linkers being sugars, hyaluronic acids derivatives, aminoacids and peptides.

As used herein, the term “biological polymer” or “biopolymer” refers to polymers produced by living organisms, or synthetic mimics of those. There are three main classes of biopolymers, classified according to the monomeric units used and the structure of the biopolymer formed: polynucleotides (RNA and DNA), which are long polymers composed of 4 or more, for example 13 or more nucleotide monomers; polypeptides, which are short polymers of amino acids; and polysaccharides, which are often linear bonded polymeric carbohydrate structures.

As used herein, the term “biodegradable polymer” refers to natural or synthetic polymers, which can undergo chemical dissolution by biological means (bacteria, enzymes, etc.) As used herein, the term “hybrid hydrogel” refers to a hydrogel comprising at least two different polymers and/or formed by the combination of at least two different polymers.

As used herein, the term “surfactant” refers to an ordered supramolecular assembly of surfactant or block copolymer molecule micelles, with translation symmetry between about 2 and about 50 nm.

As used herein, a “bioactive macromolecule” refers to a macromolecular biomolecule in an undenatured state, which still shows a conformation suited to carry on its supposed biological activity.

As used herein, a “biomolecule” refers to a naturally-occurring molecule (e.g., a compound) comprising of one or more chemical moiety(s) [“specie(s),” “group(s),” “functionality(s),” “functional group(s)” ], including but not limited to, polynucleotides (RNA and DNA), which are long polymers composed of 4 or more, for example 13 or more nucleotide monomers; polypeptides, which are short polymers of amino acids; proteins; and polysaccharides, which are often linear bonded polymeric carbohydrate structures, or a combination thereof. Examples of a macromolecule includes, an enzyme, an antibody, a receptor, a transport protein, structural protein, a prion, an antibiological proteinaceous molecule (e.g., an antimicrobial proteinaceous molecule, an antifungal proteinaceous molecule), or a combination thereof.

As used herein a “proteinaceous molecule,” proteinaceous composition,” and/or “peptidic agent” comprises a polymer formed from an amino acid, such as a peptide (i.e., about 3 to about 100 amino acids), a polypeptide (i.e., about 101 or more amino acids, such as about 50,000 or more amino acids), and/or a protein. As used herein a “protein” comprises a proteinaceous molecule comprising a contiguous molecular sequence of three amino acids or greater in length, matching the length of a biologically produced proteinaceous molecule encoded by the genome of an organism. Examples of a proteinaceous molecule include an enzyme, an antibody, a receptor, a transport protein, a structural protein, or a combination thereof. Examples of a peptide (e.g., an inhibitory peptide, an antifungal peptide) of about 3 to about 100 amino acids (e.g., about 3 to about 15 amino acids). A peptidic agent and/or proteinaceous molecule may comprise a mixture of such peptide(s) (e.g., an aliquot of a peptide library), polypeptide(s) and/or protein(s), and may also include materials such as any associated stabilizer(s), carrier(s), and/or inactive peptide(s), polypeptide(s), and/or protein(s).

### DESCRIPTION OF EXEMPLARY EMBODIMENTS

As discussed above, it may be advantageous to provide an injectable product that is biocompatible and biodegradable, and compound that could be used as to fit any wound whatever its form and/or size and/or localization. In this context, there is provided herein efficient polymerization methods able to address or accomplish this goal. In one aspect, there is provided a hybrid hydrogel comprising:

A) A first hydrogel polymer comprising monomers of formula (I):

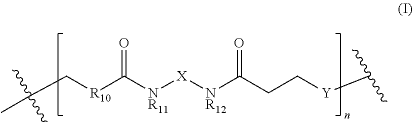

wherein
n is an integer representing the number of monomers (I) in the hydrogel polymer; for each occurrence of the bracketed structure n, Y independently represents:
   a molecular crosslinker for connecting at least a monomer of formula (I) in the framework to at least another monomer of formula (I) in another framework through a linker having the following structure:

*-R¹-L₁-R²-*;
wherein:
each occurrence of *-R¹-L₁-R²-* independently represents a responsively cleavable moiety or a non-cleavable moiety;
each occurrence of * denotes a point of attachment of the linker to a monomer of formula (I) in the hydrogel's framework;
L₁ represents a responsively cleavable covalent bond, a moiety containing a responsively cleavable covalent bond and/or a stable covalent bond;
R¹ and R² independently represent an optionally substituted C1-20 alkylenyl moiety, an optionally substituted C1-20heteroalkylenyl moiety, an optionally substituted ethenylenyl moiety, —C≡C— or an optionally substituted phenyl moiety (i.e., a moiety comprising a phenyl group that may have on one or both sides an alkylenyl or heteroalkylenyl group: in other words “optionally substituted phenyl moiety” encompasses moieties such as —C₀₋₁₀alkyl-Ph-, —C₀₋₁₀heteroalkyl-Ph-, —C₀₋₁₀alkyl-Ph-C₀₋₁₀alkyl-, —C₀₋₁₀alkyl-Ph-C₀₋₁₀heteroalkyl-, —C₀₋₁₀heteroalkyl-Ph-C₀₋₁₀alkyl-, —C₀₋₁₀heteroalkyl-Ph-C₀₋₁₀heteroalkyl-, wherein Ph may bear one or more additional substituents independently, such as halogen, C1-6alkyl, —NO₂, —CN, isocyano, —ORp, —N(Rp)2 as defined below); The aforementioned C1-20 alkylenyl, C1-20 heteroalkylenyl or ethenylenyl moiety may bear one or more substituents, independently, such as halogen or —OR where R may represent H or C1-6 alkyl; The aforementioned phenyl moiety may bear one or more substituents independently, such as halogen, C1-6alkyl, —NO₂, —CN, isocyano, —ORp, —N(Rp)2 wherein each occurrence of Rp independently represents H or C1-6alkyl;
wherein *-R¹-L₁-R²-* may independently comprise sugar derivatives such as mannose, hyaluronic acid derivatives, collagene, aminoacids or peptides;
or
O, S or a group of formula

*-R₇(R₈)-*
wherein
the * symbols denote the points of attachment of Y within the monomer backbone of formula (I);
R⁷ represents N,
R8 represents an optionally substituted C1-20 alkyl, C1-20alkenyl or C1-20alkynyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety (i.e., a moiety comprising a phenyl group that may have on one or both sides an alkyl or heteroalkyl branch or an alkylenyl or heteroalkylenyl group: in other words “optionally substituted phenyl moiety” encompasses moieties such as C₀₋₁₀alkyl-Ph, C₀₋₁₀heteroalkyl-Ph, C₀₋₁₀alkyl-Ph-C₀₋₁₀alkyl, C₀₋₁₀alkyl-Ph-C₀₋₁₀heteroalkyl, C₀₋₁₀heteroalkyl-Ph-C₀₋₁₀ alkyl, C₀₋₁₀heteroalkyl-Ph-C₀₋₁₀heteroalkyl, wherein Ph may bear one or more additional substituents independently, such as halogen, C1-6alkyl, —NO₂, —CN, isocyano, —ORp, —N(Rp)2 as defined below); Each of the foregoing C1-20 alkyl, C1-20alkenyl, C1-20alkynyl, C1-20heteroalkyl or C1-20alkylphenyl moieties may bear one or more substituents, independently, such as halogen, —OR, —CO₂R or —N(Rp)2; where R may represent H or C1-6alkyl and each occurrence of Rp may independently represent H or C1-6alkyl; The foregoing phenyl moiety may bear one or more substituents independently, such as halogen, C1-6alkyl, —NO₂, —CN, isocyano, —ORp, —N(Rp)2 wherein each occurrence of Rp independently represents H, C1-6alkyl or C1-6 alkoxy; wherein R⁸ may be optionally crosslinked to another monomer of formula (I) in another hydrogel polymer chain;
   a hyaluronic acid, alginic acid, peptide, cellulose, amino acid, sugar (for example glucose, lactose or mannose derivatives), or oligonucleotide moiety;
for each occurrence of the bracketed structure n, R₁₀ independently represents an optionally substituted C1-20 alkylenyl moiety, wherein the C1-20 alkylenyl moiety may bear one or more substituents, independently, such as halogen or —OR where R may represent H or C1-6alkyl;
for each occurrence of the bracketed structure n, R₁₁ and R₁₂ independently represent H, an optionally substituted C1-20 alkyl, C1-20alkenyl or C1-20alkynyl moiety, an optionally substituted C1-20heteroalkyl moiety, or an optionally substituted phenyl moiety; wherein each of the foregoing C1-20 alkyl, C1-20alkenyl, C1-20alkynyl or C1-20heteroalkyl moiety may bear one or more substituents, independently, such as halogen or —OR where R may represent H or C1-6alkyl, and the phenyl moiety may bear one or more substituents independently, such as halogen, C1-6alkyl, —NO₂, —CN, isocyano, —ORp, —N(Rp)2 wherein each occurrence of Rp independently represents H or C1-6alkyl;
   for each occurrence of the bracketed structure n, X independently represents an optionally substituted C1-20 alkylenyl moiety, wherein the C1-20 alkylenyl moiety may bear one or more substituents, independently, such as halogen or —OR where R may represent H or C1-6alkyl; and

B) at least a second polymer and/or hydrogel framework.

In formula (I), it is to be understood that the n bracketed structures may be the same or different.

Advantageously, the first hydrogel polymer may be composed of a succession of repeat units of formula I (no other monomer is used to make up the first hydrogel polymer structure).

Advantageously, R¹⁰ may represent CH or CH—CH₂, preferably CH.

Advantageously, the hyaluronic acid, alginic acid, peptide, cellulose, amino acid, sugar (for example glucose, lactose or mannose derivatives), or oligonucleotide moiety may be incorporated via an amino group (NH₂) naturally present on the hyaluronic acid, alginic acid, peptide, cellulose, amino acid, sugar, or oligonucleotide moiety. Alternatively, the hyaluronic acid, alginic acid, peptide, cellulose, amino acid, sugar, or oligonucleotide moiety may be chemically modified to bear an amino group, prior to incorporation in the hydrogel polymer structure, as variable Y.

In exemplary embodiments, at least one occurrence of Y in the first hydrogel polymer may bear or comprise an organosilica particle (organosilica nanoparticle or core-shell nanocapsule, wherein the organosilica matrix may be porous (preferably mesoporous) and may contain responsively cleavable bonds within the organosilica framework (in other words, the organosilica nanoparticle or core-shell nanocapsule may be degradable upon application of an external stimulus, or may be non-degradable)), as further described infra. Advantageously, at least a subset of occurrences of Y in the first hydrogel polymer bears or comprises an organosilica particle, as defined immediately above. Preferably, the organosilica particles may be functionalized so as to allow crosslinking between the first hydrogel polymers (in other words, the organosilica particles allow connecting at least a monomer of formula (I) in the framework to at least another monomer of formula (I) in another framework).

In other exemplary embodiments, organosilica material may be non-covalently embedded (e.g., mixed or dispersed) in the hybrid hydrogel network. As mentioned previously, the organosilica material may be porous (preferably mesoporous), and may be in the form of an organosilica nanoparticle or core-shell nanocapsule, as further described herein.

Advantageously, the first hydrogel polymer may be terminated by appropriate termination groups, as dictated by the chemical synthesis and reaction conditions used. For example, the first hydrogel polymer may be terminated independently at each end with H, or a starting material used in the synthesis (one of the building blocks used to make up the monomer of formula (I)).

Advantageously, n, the number of monomers (I), can be such that the mass of said hydrogel polymer may be greater than about 100 kilodaltons. The number of monomers, “n”, can be such that the mass of the hydrogel polymer of formula (I) is less than about 1000 daltons. Advantageously, the mass of the hydrogel polymer of formula (I) may range from about 300 daltons to infinite, for example from about 500 daltons to infinite. The molecular mass of the hydrogel can be considered to be infinite, on account that the hydrogel network may be completely crosslinked.

Advantageously, n may be an integer between 2 and 10000, for example between 2 and 1000, between 4 and 100, between 10 and 100, between 4 and 50, preferably between 2 and 10.

Advantageously R₁₀ may independently represent a C1-20 alkylenyl moiety, for example a C1-6 alkylenyl moiety, for example —CH₂— or —CH₂—CH₂—, advantageously —CH₂—. R11 and R12 may independently represent H or C1-C6 alkyl.

It will be understood that, in the hydrogel polymer of Formula I, each occurrence of the linker having the structure:

*-R¹-L₁-R²-*;

may independently be non-responsively cleavable (e.g., L₁ may be a stable covalent bond) or responsively cleavable (—R¹-L₁-R²— contains at least one responsively cleavable bond).

When occurrences of —R¹-L₁-R²— are responsively cleavable, they may each independently contain at least one bond (any bond) that is cleavable upon application of a particular stimulus. For example a responsively cleavable —R¹-L₁-R²— linker may contain at least one bond or moiety that may be cleaved upon a change in pH (either an increase or decrease), a change in redox potential, the presence of reduction or oxidation agent, the presence of UV, visible, infrared or near infrared light, ultrasounds, electromagnetic radiation, an enzymatic cleavage, a change in temperature, etc. Preferably “change in temperature” does not encompass large temperature increase above the decomposition temperature of the overall material containing the —R¹-L₁-R²— linker (e.g., calcination of the material). Examples of cleavable bonds envisaged in the context of the invention include, but are not limited to disulfide, diselenide, anhydride, carboxylic ester, amide, imine, acetal, ketal, urea, thiourea, hydrazine, oxyme, boronic acid derivatives such as

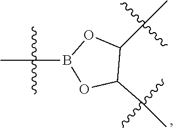 [SMILES: *B1OC(*)C(*)O1]

carbamoyl, thioketal and peptides, to name a few. Examples of cleavable moieties envisaged in the context of the invention include, but are not limited to, pH-cleavable such as

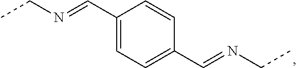

and light-cleavable moieties such as

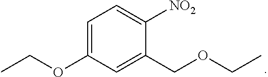 [SMILES: CCOCc1cc(OCC)ccc1[N+](=O)[O-]]

Advantageously R₁₁ and R₁₂ may independently represent H, a C1-20 alkyl, C1-20alkenyl or C1-20alkynyl moiety, a C1-20heteroalkyl-moiety, or a phenyl moiety. Advantageously R₁₁ and R₁₂ may independently represent H or C1-C6 alkyl. Advantageously R₁₁ and R₁₂ may be identical. Advantageously R₁₁ and R₁₂ may represent H.

Advantageously, X may independently represent a C1-20 alkylenyl moiety, for example a C1-6 alkylenyl moiety, for example —CH₂— or —CH₂—CH₂—, advantageously —CH₂—.

Advantageously, in the linker *-R¹-L₁-R²-*, each occurrence of R¹ and R² may be identical.

Advantageously, in the linker *-R¹-L₁-R²-*, R¹ and R² may independently represent —CH₂—, —(CH₂)₂—, —(CH₂)₃—, —(CH₂)₄—, or phenyl.

Advantageously, R¹ and R² may be identical and may each represent —CH₂—, —(CH₂)₂—, —(CH₂)₃—, —(CH₂)₄—, or phenyl.

Advantageously, when *-R¹-L₁-R²-* represents a responsively cleavable moiety, the substituent(s) on R¹ and R² may be suitably selected to facilitate the cleavage of the responsively cleavable linker L₁ when an external signal/stimulus is applied (e.g., a change in pH (either an increase or decrease), a change in redox potential, the presence of reduction or oxidation agent, the presence of UV light or near infrared light, an enzymatic cleavage, a change in temperature, etc.). For example, the substituent(s) on R¹ and R² may be selected based on their electron-withdrawing or -donating properties, to facilitate the cleavage of the linker moiety. For example, for illustrative purposes, when L may be an imine bond and R₁ and/or R₂ may be a phenyl group, the phenyl group may bear a nitro group to make the imine bond more reactive (i.e., more responsive to cleavage upon application of a suitable stimulus).

For example, L₁ may represent independently a responsively cleavable covalent bond such as:

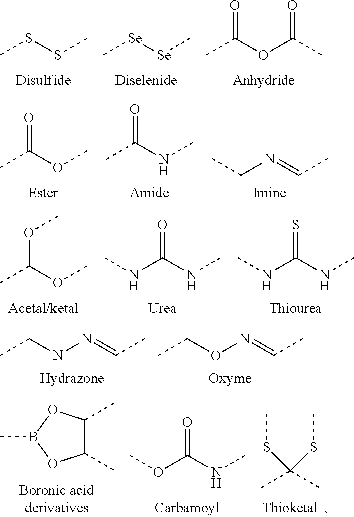

Advantageously, L₁ may independently represent or comprise a disulfide, ester, imine or hydrazone bond, preferably a disulfide bond.

Advantageously, when L₁ represents an imine bond, *-R¹-L₁-R²-* may preferably be a di-imine linker conjugated with an aromatic group such as phenyl. More preferably, *-R¹-L₁-R²-* may comprise a para di-imino phenyl moiety. Such di-imine linkers may be cleaved in acidic conditions (e.g., at pH 5-6 for 24 hours, for example pH=5.2). For example, the linker —R¹-L₁-R²— may comprise the structure

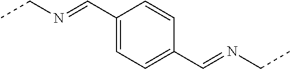

which is pH cleavable.

Advantageously, *-R¹-L₁-R²-* may independently comprise sugar derivatives such as mannose, hyaluronic acid derivatives, collagene, aminoacids or peptides; all of which may serve as degradable crosslinker.

Advantageously, *-R¹-L₁-R²-* may represent independently a responsively pH cleavable moiety of formula (III):

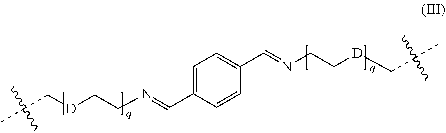

wherein each occurrence of q independently represents an integer, for example q may be an integer from 1 to 6,

D independently represents for each occurrence a C1-C3 alkylenyl moiety, or —N(Rz)- wherein Rz represents H or C1-6alkyl. As such, *-R¹-L₁-R²-* may contain more than one responsively cleavable covalent bond. In this case (linker of formula (III)), *-R¹-L₁-R²-* contains two responsively pH cleavable covalent bond (two imine bonds). Advantageously, the responsively pH cleavable moiety of formula (III) may be bound on either side to a monomer of formula (I) via a nitrogen atom (in other words, Y may be a molecular crosslinker having the structure

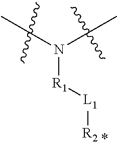

where —R¹-L₁-R²-* may have formula (III) as defined above, and * denotes the point of attachment of the molecular crosslinker to another monomer of formula (I) in the first hydrogel polymer network.

Advantageously, *-R¹-L₁-R²-* may represent independently a responsively pH cleavable moiety of formula IIIa, IIIa′ or IIIb:

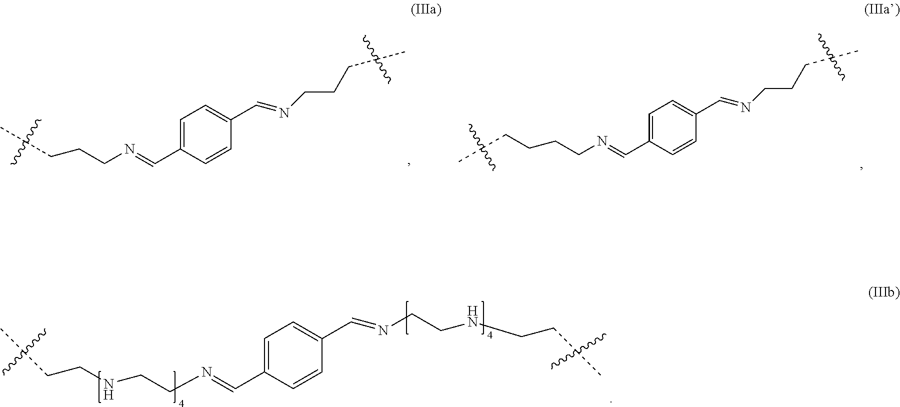

Advantageously, the responsively pH cleavable moiety of formula (IIIa), (IIIa′) or (IIIb) may be bound on either side to a monomer of formula (I) via a nitrogen atom (in other words, Y may be a molecular crosslinker having the structure

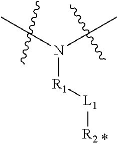

where —R¹-L₁-R²-* may have formula (IIIa), (IIIa′) or (IIIb) as defined above, and * denotes the point of attachment of the molecular crosslinker to another monomer of formula (I) in the first hydrogel polymer network.

Advantageously, L₁ or —R¹-L₁-R²-* may represent independently a light responsively cleavable group and/or a photo-responsive cleavable group. The light-responsively cleavable group and/or photo-responsive cleavable group may be any suitable light responsively cleavable group and/or photo-responsive cleavable group known from the person of ordinary skill in the art. For example, —R¹-L₁-R²-* may represent a light-induced cleavable linker having formula:

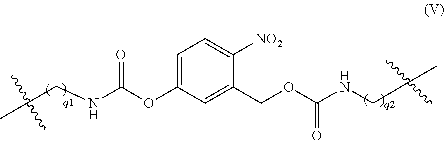

wherein q1 and q2 independently represent an integer from 1 to 6, preferably from 1 to 3. For example, q1 and q2 may both represent an integer from 1 to 6, preferably from 1 to 3, more preferably q1=q2=3. The light-sensitive linker (V) may be cleaved by irradiation with light produced by a Hg lamp. For example the light-responsive linker may comprise the structure:

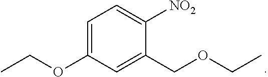 [SMILES: CCOCc1cc(OCC)ccc1[N+](=O)[O-]]

Likewise, advantageously, the light-sensitive cleavable moiety of formula (V) may be bound on either side to a monomer of formula (I) via a nitrogen atom (in other words, Y may be a molecular crosslinker having the structure

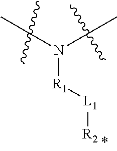

where —R¹-L₁-R²-* may have formula (V) as defined above, and * denotes the point of attachment of the molecular crosslinker to another monomer of formula (I) in the first hydrogel polymer network.

Advantageously, *-R¹-L₁-R²-* may represent independently a responsively cleavable moiety such as:

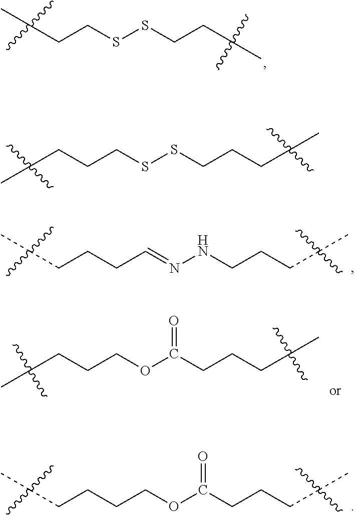

Likewise, these linkers may be bound on either side to a monomer of formula (I) via a nitrogen atom, as described above for crosslinkers III, IIIa, (IIIa′) and IIIb.

Advantageously, L₁ and *-R¹-L₁-R²-* may independently be a stable covalent bond or moiety, respectively (i.e., which is not cleaved under the conditions in which it is used/intended), for example it may be any stable bond or moiety known to the person of ordinary skill in the art and adapted to cross-link monomer and/or polymer frameworks. It may be for example a C1-20 alkylenyl moiety or C1-20 heteroalkylenyl moiety, for example a C1-6 alkylenyl or C1-6 heteroalkylenyl moiety, polyglycols, or lipids. When L₁ and *-R¹-L₁-R²-* represent a stable covalent bond or moiety, for each iteration of the monomer (I), the first hydrogel is said to be non-degradable. For example, *-R¹-L₁-R²-* may represent:

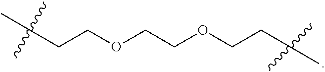 [SMILES: *CCOCCOCC*]

Advantageously, in the group of formula *-R₇(R₈)-*, R⁷ may be N and R⁸ may represent an optionally substituted C1-20 alkyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety (i.e., a moiety comprising a phenyl group that may have on one or both sides an alkyl or heteroalkyl branch or alkylenyl or heteroalkylenyl group: in other words “optionally substituted phenyl moiety” encompasses moieties such as C₀₋₁₀alkyl-Ph, C₀₋₁₀heteroalkyl-Ph, C₀₋₁₀alkyl-Ph-C₀₋₁₀alkyl, C₀₋₁₀alkyl-Ph-C₀₋₁₀heteroalkyl, C₀₋₁₀heteroalkyl-Ph-C₀₋₁₀alkyl, C₀₋₁₀heteroalkyl-Ph-C₀₋₁₀ heteroalkyl, wherein Ph may bear one or more substituents independently, such as halogen, C1-6alkyl, —NO₂, —CN, isocyano, —ORp, —N(Rp)2 as defined below); wherein each of the foregoing C1-20 alkyl, C1-20heteroalkyl or C1-20alkylphenyl moieties may bear one or more substituents, independently, such as halogen, —OR, —CO₂R or —N(Rp)2 where R may represent H or C1-6alkyl, and each occurrence of Rp may independently represent H or C1-6alkyl; and the phenyl moiety may bear one or more substituents independently, such as halogen, C1-6alkyl, —NO2, —CN, isocyano, —ORp, —N(Rp)2 wherein each occurrence of Rp independently represents H, C1-6alkyl or C1-6 alkoxy.

Advantageously, Y may represent a group of formula *-N(R⁸)-*, wherein R⁸ may represent the residue of the corresponding amino acid H₂NR⁸. For example, gamma-aminobutyric acid may be used, and Y may represent *-N[(CH₂)₃CO₂H]-*.

Advantageously, in the group of formula *-R₇(R₈)-*, R⁷ may be N and R⁸ may represent a C1-C6 alkyl substituted with a carboxyl moiety, a C1-C6 alkyl substituted with one or more hydroxyl groups, C1-C6 alkoxy, C1-C6 alkyl substituted with —N(Rp)2 wherein each occurrence of Rp independently represents a C1-6alkyl.

Advantageously, in the group of formula *-R₇(R₈)-*, R⁸ may represent a C1-C6 alkyl substituted with —N(Rp)2 wherein each occurrence of Rp independently represents a C1-6alkyl; for example a C1-C2 alkyl substituted with-N(Rp)2 wherein each occurrence of Rp independently represents a C1-2alkyl. R⁸ may represent a C2 alkyl substituted with-N(Rp)2 wherein each occurrence of Rp independently represents a C1alkyl. For example R⁸ mar represent —(CH₂)NMe₂.

Advantageously, in the group of formula *-R₇(R₈)-*, R⁷ may be N and R⁸ may represent R⁸ may represent a C2 alkyl substituted with-N(Rp)2 wherein each occurrence of Rp independently represents a C1alkyl. For example R⁸ mar represent —(CH₂)NMe₂.

Advantageously, in the group of formula *-R₇(R₈)-*, R⁷ may be may be N, and R⁸ may represent independently from other occurrences of R8 a C1-20alkylphenyl moiety optionally substituted with one or more —OR wherein R may represent H or C1-6alkyl. For example, R⁸ may represent independently from other occurrences of R8 a C1-6alkylphenyl moiety optionally substituted with one or more —OR wherein R may represent H or C1-6alkyl. For example, R⁸ may represent independently from other occurrences of R8 a C1-6alkyl moiety bearing a catechol moiety.

Advantageously, in the group of formula *-R₇(R₈)-*, R⁷ may be may be N, and R⁸ may be independently a group of following formula:

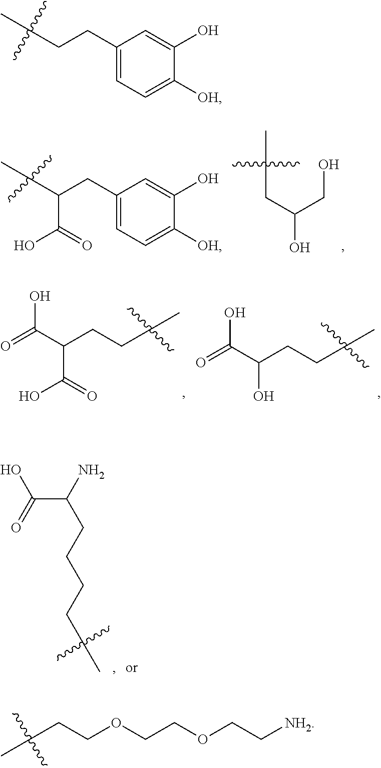

Advantageously, the hybrid hydrogels of the invention may carry biologicals molecules. In particular, Y may advantageously represent a moiety selected from the group comprising hyaluronic acid, alginic acid, amino acid, peptide, cellulose, sugar (for example glucose, lactose or mannose derivatives) and oligonucleotide moieties.

Advantageously, the hyaluronic acid derivatives may be any hyaluronic acid derivatives known to the person of ordinary skill in the art. It may be for example any commercially available hyaluronic acid derivatives, for example a hyaluronic acid derivative disclosed in Voigt J et al. “Hyaluronic acid derivatives and their healing effect on burns, epithelial surgical wounds, and chronic wounds: a systematic review and meta-analysis of randomized controlled trials.” Wound Repair Regen. 2012 May-June; 20(3):317-31 [30]. For example, a hyaluronic acid moiety may be introduced in monomers of formula (I) as Y=*-N(R₈)-*, via a hydrolysed version of the naturally occurring hyaluronic acid molecule (e.g., hydrolysis of —NHAc moiety into —NH₂).

Advantageously, the alginic acid derivatives may be any alginic acid derivatives known to the person of ordinary skill in the art. It may be, for example, commercially available alginic acid or alginic acid sodium salt, from different sources and of any available molecular weight, such as alginic acid sodium salt derived from brown algae, including Laminaria hyperborea, Laminaria digitata, Laminaria japonica, Ascophyllum nodosum, and Macrocystis pyrifera, or obtained from genetic engineered bacteria. It may be chemically modified to improve adhesion or biocompatibility, for example through oxidation, functionalization or conjugation with small molecules, for example Dodecylamine, or with biomolecules, such as peptides, cellulose or sugars, as disclosed for example in K. J. Lee, D. J. Mooney, “Alginate: properties and biomedical applications”, Prog Polym Sci., 2012 January; 37(1) 106-126. [31]

Advantageously, when Y represents an amino acid it may be any amino acid known to the person of ordinary skill in the art. It may be for example D or L amino acid. It may be for example amino acid selected from the group comprising alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. It may also be gamma aminobutyric acid.

Advantageously, when Y represents a peptide moiety, it may be peptide moiety comprising for 3 to 20 amino acids, for example 3 to 5 amino acids.

Advantageously, when Y represents a sugar moiety (carbohydrate moiety), it may be any sugar known to the person of ordinary skill in the art and adapted to be linked to a polymer framework. It may be for example a sugar selected from the group comprising Arabinose, Fructose, Galactose, Glucose, Lactose, Inositol, Mannose, Ribose, Trehalose and Xylose, preferably glucose, lactose or mannose. Advantageously, these sugars may be functionalized with an amino-containing moiety, for proper incorporation of the sugar moiety as Y into the monomer of formula (I).

Advantageously, when Y represents an oligonucleotide moiety it may be derived from any oligonucleotide known to the person of ordinary skill in the art and adapted to be linked to a polymer framework. It may be for example an oligonucleotide moiety comprising from 2 to 25 Deoxyribonucleic acid and/or Ribonucleic acid. Advantageously, the oligonucleotide moiety may be functionalized with an amino-containing moiety, for proper incorporation of the oligonucleotide moiety as Y into the monomer of formula (I).

Advantageously, the at least a second polymer and/or hydrogel framework may be a polysaccharide-based hydrogel, preferably an alginate-based hydrogel. In the present disclosure, the alginate may be any alginate known to the person of ordinary skill in the art. It may be for example commercially available alginate, for example extracted from brown algae (Phaeophyceae), including Laminaria hyperborea, Laminaria digitata, Laminaria japonica, Ascophyllum nodosum, and Macrocystis pyrifera by treatment with aqueous alkali solutions, typically with NaOH, and/or alginate provided by bacterial synthesis, for example from Azotobacter and Pseudomonas. It may be, for example alginate as described in Kuen Yong Lee et al. “Alginate: properties and biomedical applications” Prog Polym Sci. 2012 January; 37(1): 106-126 [31].

Advantageously, an exemplary alginate hydrogel useable within the context of the invention may be of formula (II):

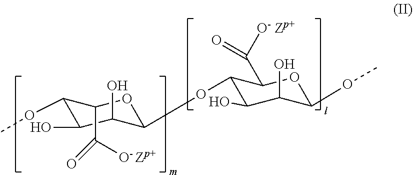

wherein each occurrence of Z independently represents a counterion such as Ca, Mg, Na, K, Li, Rb and m, l, p are independently integers.

In formula (II), it is to be understood that the m and l bracketed structures do not necessarily represent two distinct blocks m and l, but rather that the alginate polymer contains m bracketed monomers “m” and l bracketed monomers “l”, the distribution of which may vary from one alginate to another.

Advantageously, the sum of m+l may be such that the mass of said alginate hydrogel may be greater than about 500.000 daltons and/or less than about 20.000 daltons.

Advantageously, the mass of said alginate hydrogel may range from 30 to 400 kdaltons.

Advantageously, m may be an integer between 2 and 1000, for example between 10 and 1000.

Advantageously, 1 may be an integer between 2 and 1000, for example between 10 and 1000.

Advantageously, the ratio m/1 may be from 0.01 to 100, for example between 0.1 and 10.

Typically, hydrogels according to the invention may be prepared using an aqueous solution of alginate ranging from 0% (pure water) to about 2% (very concentrated alginate).
Advantageously, hybrid hydrogels combining a first hydrogel of formula (I) and an alginate hydrogel are particularly preferred. Any ratio/concentration of alginate polymer in the hybrid hydrogel can be used. As such, hybrid hydrogels according to the present invention may have a dry alginate content from 0.01% to 99.99% by weight in respect to the dry weight of the hybrid hydrogel. The content of alginate in the hybrid hydrogel will depend on the intended application, and the desired gel properties, notably the physico-mechanical properties of the hydrogel (for example, hydrogel elasticity and/or mechanical stability).
For applications where hydrogel elasticity and/or mechanical stability is important, a relatively low content of alginate will be preferred. For example, a dry alginate content from 0.01% to 20% by weight in respect to the dry weight of the hybrid hydrogel may be used, for example 0.01% to 15%, 0.01% to 10%, 0.01% to 8%, 0.01% to 5%, 0.01% to 4%, 0.01% to 3%, 0.01% to 2% dry weight of alginate in respect to the dry weight of the hybrid hydrogel.

Advantageously, the final percentage in weight of the alginate over the entire mass of the hydrogel may be between 0 and 3%. Advantageously, the percentage in weight of alginate in respect to the dry weight of the hydrogel may be between 0 and 20%. Advantageously, the ratio between the mass of the first hydrogel of formula (I) and the alginate can range from about 5 to nearly infinite (nearly the first hydrogel of formula (I) may be present).

Advantageously, the hybrid hydrogel comprising a first hydrogel of formula (I) and alginate hydrogel have improved formation properties. The supramolecular gelation of the alginate upon Ca addition create a network that facilitates the covalent cross linking of the polyamidoamines and most important renders the gel already semisolid (cf. FIG. 12 which shows that alginate has a lower module to PAAm while PAAm+alginate has similar module but higher linear elastic range, i.e. it is more resistant and less fragile). The advantage of the hybrid hydrogel of the invention is that it has rheological properties much more performant than the alginate or the hydrogel alone. In particular, it has been surprisingly demonstrated that the hybrid hydrogel according to the invention has higher polymerization capacities and can be formed in-situ, for example via Michael-type addition reaction under physiological conditions from simple mixing of the monomers in aqueous solution through the formation of amine bonds.

Advantageously, contrary to known hydrogels and/or commonly used hydrogels, hybrid hydrogels of the invention may be obtained and formed under physiological conditions, for example in aqueous solution at a temperature around 37° C. and a pH about 7.4.

The hybrid hydrogels according to the invention may advantageously be associated with non covalently or covalently with organosilica material for example in the form of particles (organosilica nanoparticles or core-shell nanocapsules), wherein the organosilica matrix may be porous (preferably mesoporous) and may contain responsively cleavable bonds L₂ or responsively cleavable linkers #-R³-L₂-R⁴-# within the organosilica framework (in other words, the organosilica nanoparticles or core-shell nanocapsules may be degradable upon application of an external stimulus, or may be non-degradable)), as further described infra. The particles may be mixed in a solution of the first hydrogel polymer of formula (I) and the second polymer (preferably a polysaccharide-based hydrogel, more preferably an alginate-based hydrogel polymer) composing the hybrid hydrogel, followed by gelation of the hybrid hydrogel. As such, the organosilica particles may be embedded within the hydrogel matrix, and may be released upon degradation of the hybrid hydrogel framework, for example if the hydrogel is degradable.
The hybrid hydrogels according to the invention may be advantageously functionalized, for example with organosilica material for example in the form of particles (organosilica nanoparticles or core-shell nanocapsules), wherein the organosilica matrix may be porous (preferably mesoporous) and may contain responsively cleavable bonds L₂ or responsively cleavable linkers #-R³-L₂-R⁴-# within the organosilica framework (in other words, the organosilica nanoparticles or core-shell nanocapsules may be degradable upon application of an external stimulus, or may be non-degradable)), as further described infra.

Advantageously, at least a subset of occurrences of Y in the first hydrogel polymer may represent *-N(R⁸)-* wherein R⁸ represents a C1-20alkyl or C1-20heteroalkyl moiety, preferably C1-6alkyl or C1-6heteroalkyl, most preferably C1-6alkyl, bearing an organosilica nanoparticle, preferably the organosilica matrix may be porous, most preferably mesoporous, and may contain responsively cleavable bonds L₂ or responsively cleavable linkers #-R³-L₂-R⁴-# within the organosilica framework (R₃, R₄ and L₂ are as defined below). Advantageously, when R⁸ comprises an organosilica particle, preferably an organosilica nanoparticle, it may be bound on either side to a monomer of formula (I) via a nitrogen atom (in other words, Y may be a molecular crosslinker having the structure

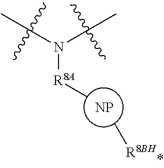

wherein R^{8A} and R^{8B} independently represent a C1-10alkyl or C1-10heteroalkyl moiety, preferably C1-6alkyl or C1-6heteroalkyl; NP denotes an organosilica nanoparticle; and * denotes the point of attachment of the molecular crosslinker to another monomer of formula (I) in the first hydrogel polymer network).

It is understood that the above is but a non-limiting illustration of how organosilica material can be embedded into the hydrogel. The anchoring point of the organosilica material can be anywhere in the gel: covalently or non-covalently. The organosilica material, which may be porous (preferably mesoporous), and may be in the form of an organosilica nanoparticle or core-shell nanocapsule, as further described herein, may be dispersed/mixed in the hydrogel. Alternatively or additionally, the organosilica material may be covalently bound to a compound/moiety that is dispersed or covalently conjugated to the hydrogel polymer (for example, the organosilica material may be covalently bound to through the carboxylate of a dopamine moiety present in the gel).

The organosilica material, whether covalently conjugated to the hydrogel polymer of formula I or non-covalenty embedded (mixed/dispersed) within the hydrogel network, may be nanometric or micrometric in size.

For example, the organosilica material may be organosilica particles, preferably porous organosilica particles, most preferably mesoporous organosilica particles, with a diameter ranging from 1 nanometer to 10 micrometers. In exemplary embodiments, the organosilica material may be organosilica nanoparticles, preferably porous organosilica nanoparticles, most preferably mesoporous organosilica nanoparticles, with a diameter ranging from 1 nanometer to 999 nanometers, for example from 1 to 500 nm. As non-limiting illustrative examples, organosilica nanoparticles about 20, 30, 45, 60, 100, 250, 500 nm may be used, preferably organosilica nanoparticles with a porous organosilica matrix, most preferably mesoporous organosilica matrix.

In additional exemplary embodiments, the organosilica material may be organosilica core-shell capsules, preferably with porous organosilica matrix, most preferably with mesoporous organosilica matrix, with a diameter ranging from 1 nanometer to 999 nanometers, for example from 1 to 500 nm. As non-limiting illustrative examples, organosilica core-shell nanocapsules about 60, 100, 120 nm may be used, preferably organosilica core-shell nanocapsules with a porous organosilica matrix, most preferably a mesoporous organosilica matrix.

However, a wide range of size of organosilica material may be used in the context of the present invention. The reader will select appropriate organosilica material size depending on the intended application.

Organosilica materials are well known, as well as method for preparing them, such as sol gel chemistry-based methods. The organosilica material, optionally in the form of nanoparticles, may be degradable as described in WO 2015/107087, the entire contents of which are hereby incorporated by reference herein. The reader may refer to the teachings of this document for guidance as to how to prepare such degradable/disintegrable organosilica materials.

Advantageously, at least a subset of occurrences of Y in the first hydrogel polymer may represent *-N(R⁸)-* wherein R⁸ represents a C1-20alkyl or C1-20heteroalkyl moiety, preferably C1-6alkyl or C1-6heteroalkyl, most preferably C1-6alkyl, bearing an organosilica core/shell nanocapsule, preferably the organosilica matrix may be porous, most preferably mesoporous, and may contain responsively cleavable bonds L₂ or responsively cleavable linkers #-R³-L₂-R⁴-# within the organosilica framework (R₃, R₄ and L₂ are as defined below). Preferably, the organosilica core/shell nanocapsule may be degradable/disintegrable in that its shell framework contains Si adjacent sites covalently bound via a responsively cleavable linker, as described in WO 2015/189402, the entire contents of which are hereby incorporated by reference herein. Advantageously, the organosilica core/shell nanocapsule may encapsulate a bioactive macromolecule or bioactive macromolecule cluster, and/or another molecule of interest that may or may not have biological activity and/or pharmaceutical or cosmetic activity. Advantageously, the bioactive macromolecule or bioactive macromolecule cluster encapsulated within the nanocapsule may be in active conformation (i.e., in a biologically active form). The reader may refer to the teachings of WO 2015/189402 for guidance as to how to prepare such degradable/disintegrable organosilica nanocapsules.

Briefly, such nanocapsules may be prepared by a method comprising steps of:
I. Producing a water-in-oil emulsion from (i) a solution of a suitable surfactant and alcohol in a suitable organic solvent, and (ii) an aqueous solution of a bioactive macromolecule or bioactive macromolecule clusters and/or another molecule of interest, a silane precursor Si(Z^{A})₄ and a selected precursor having the structure (Z)₃Si—R³-L2-R⁴—Si(Z)₃;
II. Stirring the water-in-oil emulsion obtained in step I) under alkaline conditions; thereby coating the bioactive macromolecule or bioactive macromolecule clusters with an organosilica sol-gel mixture obtained by hydrolysis-condensation of silicon alkoxide; and
III. Adding a suitable organic solvent, thereby precipitating the nanoencapsulated bioactive macromolecules or bioactive macromolecule clusters and/or other molecule of interest;
   wherein
each occurrence of Z and Z^{A} independently represents a hydrolysable or nonhydrolyzable group, provided that on each occurrence of Si of the precursor (Z)₃Si—R³-L2-R⁴—Si(Z)₃, at least one occurrence of Z represents a hydrolysable group, and at least two occurrences of Z^{A} in the the precursor Si(Z^{A})₄ independently represent a hydrolysable group; wherein (i) when Z or Z^{A} represents a nonhydrolyzable group, it may be selected from the group comprising an optionally substituted C1-20alkyl, C2-20alkenyl or C2-20alkynyl moiety, an optionally substituted C1-20heteroalkyl, C2-20heteroalkynyl or C2-20heteroalkynyl moiety, or an optionally substituted phenyl moiety; wherein the substituents on the phenyl, alkyl, alkenyl, alkynyl, heteroalkyl, heteroalkenyl and heteroalkynyl moieties may be, independently, substituents such as halogen, —NO₂, —CN, isocyano, C1-6alkoxy, an oxirane/epoxyde moiety, —N(R)₂ wherein each occurrence of R may be independently selected from the group comprising H or C1-6alkyl; and (ii) when X or X^{A} represents a hydrolysable group, it may be selected from the group comprising a C1-6alkoxy, C1-6acyloxy, halogen or amino moiety; and
   R₃, R₄, L₂ and #, are as defined generally and in any variants herein.

Likewise, advantageously, when R⁸ comprises an organosilica core/shell nanocapsule, it may be bound on either side to a monomer of formula (I) via a nitrogen atom (in other words, Y may be a molecular crosslinker having the structure

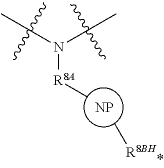

wherein R^{8A} and R^{8B} independently represent a C1-10alkyl or C1-10heteroalkyl moiety, preferably C1-6alkyl or C1-6heteroalkyl; NP denotes an organosilica core/shell nanocapsule; and * denotes the point of attachment of the molecular crosslinker to another monomer of formula (I) in the first hydrogel polymer network).

Advantageously, the aforementioned organosilica material for example in the form of particles (organosilica nanoparticles or core-shell nanocapsules), may be chemically modified to bear an amino-containing tether group at the outer surface, prior to incorporation in the first hydrogel polymer structure, as variable Y (cf. crosslinker *-R^{8A}-NP-R^{8B}-* mentioned above). Such functionalization may be effected by any suitable ways known in the art.

For example, such functionalization may be carried out by reacting organosilica material for example in the form of particles (e.g., organosilica nanoparticles or core-shell nanocapsules), with a silylated starting material (W)₃Si—R⁸—N(Rp)₂; each occurrence of W independently represents a hydrolysable group selected from the group comprising a C1-6 alkoxy, C1-6 acyloxy, halogen or an amino moiety; R⁸ represents an optionally substituted C1-20 alkyl, C2-20 alkenyl or C2-20 alkynyl moiety, an optionally substituted C1-20 heteroalkyl, C2-20 heteroalkynyl or C2-20 heteroalkynyl moiety, or an optionally substituted phenyl moiety (i.e., a moiety comprising a phenyl group that may have on one or both sides an alkyl or heteroalkyl branch or an alkylenyl or heteroalkylenyl group: in other words “optionally substituted phenyl moiety” encompasses moieties such as C₀₋₁₀alkyl-Ph, C₀₋₁₀heteroalkyl-Ph, C₀₋₁₀alkyl-Ph-C₀₋₁₀alkyl, C₀₋₁₀alkyl-Ph-C₀₋₁₀heteroalkyl, C₀₋₁₀heteroalkyl-Ph-C₀₋₁₀alkyl, C₀₋₁₀heteroalkyl-Ph-C₀₋₁₀heteroalkyl, wherein Ph may bear one or more additional substituents independently, such as halogen, C1-6alkyl, —NO₂, —CN, isocyano, —ORp, —N(Rp)₂ as defined below). The substituents on the aforementioned phenyl, alkyl, alkenyl, alkynyl, heteroalkyl, heteroalkenyl and heteroalkynyl moieties may be independently selected from the group comprising halogen, —NO₂, —CN, isocyano, C1-6 alkoxy, an oxirane/epoxyde moiety, —N(R)₂ wherein each occurrence of R is independently selected from the group comprising H or C1-6 alkyl; and each occurrence of Rp independently represents H or C1-6 alkyl. Advantageously, each occurrence of W may independently represent Cl, -OMe, -OEt, -OiPr or -OtBu. Advantageously, R⁸ may represent a C1-20alkyl or C1-20heteroalkyl moiety, preferably C1-6alkyl or C1-6heteroalkyl, most preferably C1-6alkyl.

Alternatively, such functionalization may be carried out by preparing the organosilica material for example in the form of particles (organosilica nanoparticles or core-shell nanocapsules), with a silylated starting material (W)₃Si—R⁸—N(Rp)₂ as defined above, under conventional sol gel chemistry conditions.

As a result, organosilica material for example in the form of particles (organosilica nanoparticles or core-shell nanocapsules) bearing —R⁸—N(Rp)₂ tethers at the outer surface will be obtained, and may be used for incorporation in a subset of monomers of formula (I) of the first hydrogel polymer of the hybrid hydrogels of the invention, as variable Y (cf. crosslinker *-R^{8A}-NP-R^{8B}-* mentioned above). If at least one occurrence of Rp differs from H, the —R⁸—N(Rp)₂ tethers may be first deprotected to yield —R⁸—NH₂ tethers prior to proceeding with the functionalization of the organosilica material.

Advantageously, at least a subset of occurrences of Y may further comprise a core/shell nanocapsule, advantageously an organosilica core/shell nanocapsule, preferably the shell organosilica matrix may be porous, most preferably mesoporous, preferably the shell matrix may additionally degradable/disintegrable, with a bioactive macromolecule or bioactive macromolecule cluster encapsulated within said nanocapsule. The nanocapsule may alternatively or additionally contain another molecule of interest that may or may not have biological activity and/or pharmaceutical or cosmetic activity.

Advantageously, at least a subset of occurrences of Y may comprise a nanoencapsulated molecule or bioactive macromolecule or biomacromolecule cluster comprising
a. a nanocapsule, having a core/shell structure, and
b. a molecule of interest or bioactive macromolecule or bioactive macromolecule cluster encapsulated within said nanocapsule.

Advantageously, the shell of said nanocapsule may be made of hybrid organosilica material comprising a three-dimensional framework of Si—O bonds, wherein at least a subset of Si atoms in the material's framework are connected to at least another Si atom in the framework through a linker having the following structure:

#-R³-L₂-R⁴-#;

wherein:
each occurrence of # denotes a point of attachment to a Si atom in the hybrid organosilica material's framework;
L₂ represents a responsively cleavable covalent bond or a stable bridging ligand; preferably a responsively cleavable covalent bond; and
R³ and R⁴ independently represent an optionally substituted C1-20 alkylenyl moiety, an optionally substituted C1-20 heteroalkylenyl moiety, an optionally substituted ethenylenyl moiety, —C≡C— or an optionally substituted phenyl moiety (i.e., a moiety comprising a phenyl group that may have on one or both sides an alkylenyl or heteroalkylenyl group: in other words “optionally substituted phenyl moiety” encompasses moieties such as —C₀₋₁₀alkyl-Ph-, —C₀₋₁₀heteroalkyl-Ph-, —C₀₋₁₀alkyl-Ph-C₀₋₁₀alkyl-, —C₀₋₁₀alkyl-Ph-C₀₋₁₀heteroalkyl-, —C₀₋₁₀heteroalkyl-Ph-C₀₋₁₀alkyl-, —C₀₋₁₀heteroalkyl-Ph-C₀₋₁₀heteroalkyl-, wherein Ph may bear one or more substituents independently, such as halogen, C1-6alkyl, —NO₂, —CN, isocyano, —ORp, —N(Rp)₂ as defined below); wherein the C1-20alkylenyl, C1-20 heteroalkylenyl or ethenylenyl moiety may bear one or more substituents, independently, such as halogen or —OR where R may represent H or C1-6alkyl, and the phenyl moiety may bear one or more substituents independently, such as halogen, C1-6alkyl, —NO₂, —CN, isocyano, —OR^{p}, —N(R^{p})₂ wherein each occurrence of R^{p} independently represents H or C1-6alkyl.
   It is to be understood that the definition of R₃, R₄, L₂ and # above, and variants detailed below, also apply to the organosilica matrix making up the organosilica nanoparticles mentioned before (plain nanoparticles), that may be incorporated into the first hydrogel framework as Y variable.

It will be understood that, in the hydrogel polymer of Formula I, each occurrence of the linker having the structure:

*-R³-L₂-R⁴-*;

may independently be non-responsively cleavable (e.g., L₂ may be a stable bridging ligand or a covalent bond) or responsively cleavable (—R³-L₂-R⁴— contains at least one responsively cleavable bond).

When occurrences of —R³-L₂-R⁴ are responsively cleavable, they may each independently contain at least one bond (any bond) that is cleavable upon application of a particular stimulus. For example a responsively cleavable —R³-L₂-R⁴— linker may contain at least one bond or moiety that may be cleaved upon a change in pH (either an increase or decrease), a change in redox potential, the presence of reduction or oxidation agent, the presence of UV, visible, infrared or near infrared light, ultrasounds, electromagnetic radiation, an enzymatic cleavage, a change in temperature, etc. Preferably “change in temperature” does not encompass large temperature increase above the decomposition temperature of the overall material containing the —R³-L₂-R⁴— linker (e.g., calcination of the material). Examples of cleavable bonds envisaged in the contect of the invention include, but are not limited to disulfide, diselenide, anhydride, carboxylic ester, amide, imine, acetal, ketal, urea, thiourea, hydrazine, oxyme, boronic acid derivatives such as

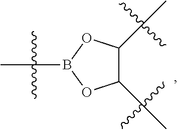 [SMILES: *B1OC(*)C(*)O1]

carbamoyl, thioketal and peptides, to name a few.

Advantageously, L₂ may be any moiety that contains a responsively cleavable covalent bond, which can be cleaved upon exposure to a determined stimulus. As non limiting examples, L₂ may represent a responsively cleavable covalent bond such as:

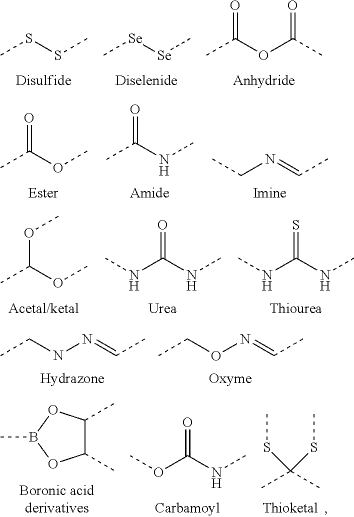

peptide moieties, light-responsively cleavable moieties, pH-responsively cleavable moieties, in any variant described supra for —R¹-L₁-R²— type linkers.

Advantageously, when L₂ represents an imine bond, #-R³-L₂-R⁴-# may preferably be a di-imine linker conjugated with an aromatic group such as phenyl. More preferably, #-R³-L₂-R⁴-# may comprise a para di-imino phenyl moiety. As discussed above, such di-imine linkers may be cleaved in acidic conditions (e.g., at pH 5-6 for 24 hours, for example pH=5.2) thereby leading to disintegration of the organosilica particles. For example, the linker —R³-L₂-R⁴— may comprise the structure

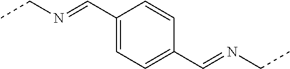

which is pH cleavable.
Advantageously, #-R³-L₂-R⁴-# may represent independently a responsively pH cleavable moiety of formula (III):

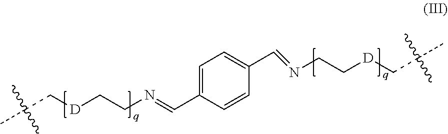

wherein q is an integer, for example q may be equal to 1 to 6,

D independently represents for each occurrence a C1-C3 alkylenyl moiety, or —N(Rz)- wherein Rz represents H or C1-6alkyl. As such, *-R³-L₂-R⁴-* may contain more than one responsively cleavable covalent bond. In this case (linker of formula (III)), *-R³-L₂-R^{4-*}contains two responsively pH cleavable covalent bond (two imine bonds).

Advantageously, #-R³-L₂-R⁴-# may represent independently a responsively pH cleavable moiety of formula IIIa, IIIa′ or IIIb:

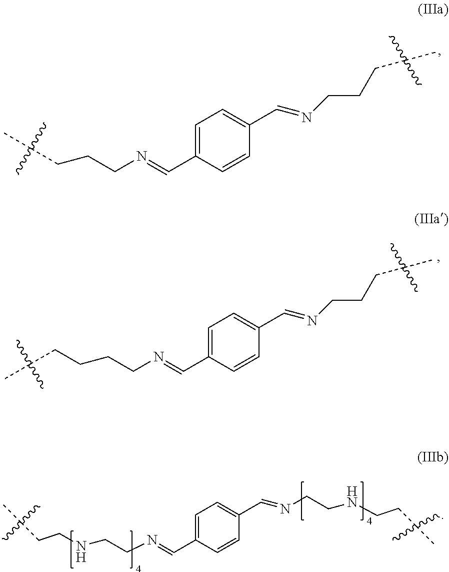

Advantageously, L₂ or #-R³-L₂-R⁴-# may represent independently a light responsively cleavable group and/or a photo-responsive cleavable group. The light-responsively cleavable group and/or photo-responsive cleavable group may be any suitable light responsively cleavable group and/or photo-responsive cleavable group known from the person of ordinary skill in the art. For example, it may be a group that can be cleaved upon application of UV, visible, infrared or near infrared irradiation. For example, #-R³-L₂-R⁴-# may represent a light-sensitive linker having formula:

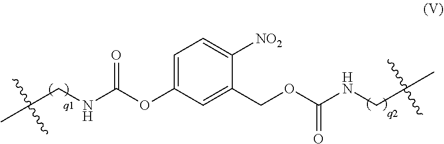

wherein q1 and q2 independently represent an integer from 1 to 6, preferably from 1 to 3. For example, q1 and q2 may both represent an integer from 1 to 6, preferably from 1 to 3, more preferably q1=q2=3. The light-sensitive linker (V) may be cleaved by irradiation with light produced by a Hg lamp. For example the light-responsive linker may comprise the structure:

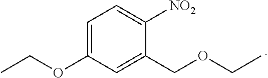 [SMILES: CCOCc1cc(OCC)ccc1[N+](=O)[O-]]

Advantageously, #-R³-L₂-R⁴-# may represent independently a responsively cleavable moiety such as

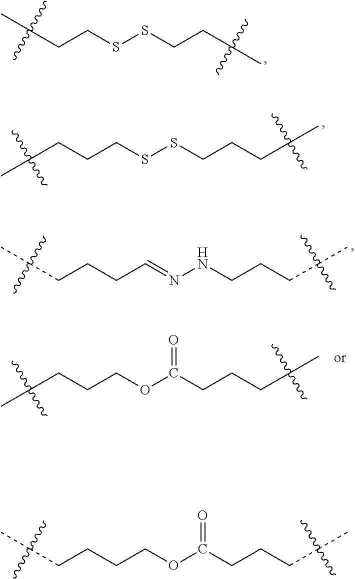

Preferably, L₂ may represent a responsively cleavable covalent bond selected from the group comprising disulfide, diselenides, imine, amide, ester, urea, hydrazone or thiourea; preferably disulfide, imine (preferably #-R³-L₂-R⁴-# may comprise a para di-imino phenyl moiety), ester, or hydrazone; more preferably disulfide.

Advantageously, the bioactive macromolecule or bioactive macromolecule cluster encapsulated within the nanocapsule may be in active conformation (i.e., in a biologically active form).

Advantageously, the bioactive macromolecule or bioactive macromolecule cluster encapsulated within the nanocapsule may be in a undenatured state.

Advantageously, the bioactive macromolecule or bioactive macromolecule cluster encapsulated within the nanocapsule may remain in a folded position and retain an active conformation.

Advantageously, in the linker #-R³-L₂-R⁴-#, each occurrence of R³ and R⁴ may be identical.

Advantageously, in the linker #-R³-L₂-R⁴-#, R³ and R⁴ may be any organic radical from any commercially available silylated derivative suitable for sol-gel chemistry. For example R³ and R⁴ may independently represent-CH₂—, —(CH₂)₂—, —(CH₂)₃—, —(CH₂)₄—, or phenyl.

Advantageously, the substituent(s) on R³ and R⁴ may be suitably selected to facilitate the cleavage of the responsively cleavable linker L₂ when an external signal/stimulus is applied (e.g., a change in pH (either an increase or decrease), a change in redox potential, the presence of reduction or oxidation agent, the presence of UV light or near infrared light, an enzymatic cleavage, a change in temperature, etc.). For example, the substituent(s) on R³ and R⁴ may be selected based on their electron-withdrawing or -donating properties, to facilitate the cleavage of the linker moiety. For example, for illustrative purposes, when L may be an imine bond and R₁ and/or R₂ may be a phenyl group, the phenyl group may bear a nitro group to make the imine bond more reactive (i.e., more responsive to cleavage upon application of a suitable stimulus).

Advantageously the nanocapsule outer surface may comprise one or more groups of formula

#-R⁵R⁶
wherein
each occurrence of # denotes a point of attachment to a Si atom at the outer surface of the hybrid organosilica material's framework;
R⁵ represents an optionally substituted C1-20alkylenyl moiety, an optionally substituted C1-20heteroalkylenyl moiety, an optionally substituted ethenylenyl moiety, —C≡C— or an optionally substituted phenyl moiety (i.e., a moiety comprising a phenyl group that may have on one or both sides an alkylenyl or heteroalkylenyl group: in other words “optionally substituted phenyl moiety” encompasses moieties such as —C₀₋₁₀alkyl-Ph-, —C₀₋₁₀heteroalkyl-Ph-, —C₀₋₁₀alkyl-Ph-C₀₋₁₀alkyl-, —C₀₋₁₀alkyl-Ph-C₀₋₁₀heteroalkyl-, —C₀₋₁₀heteroalkyl-Ph-C₀₋₁₀alkyl-, —C₀₋₁₀heteroalkyl-Ph-C₀₋₁₀heteroalkyl-, wherein Ph may bear one or more substituents independently, such as halogen, C1-6alkyl, —NO₂, —CN, isocyano, —ORp, —N(Rp)₂ as defined below). The aforementioned C1-20alkylenyl, C1-20heteroalkylenyl or ethenylenyl moiety may bear one or more substituents, independently, such as halogen or —OR where R may represent H or C1-6alkyl, and the phenyl moiety may bear one or more substituents, independently, such as halogen, C1-6alkyl, —NO2, —CN, isocyano, —ORp, —N(Rp)2 wherein each occurrence of Rp independently represents H or C1-6alkyl;
R⁶ represents —OR, —SR or —N(Rf)₂; preferably —N(Rf)₂; wherein each occurrence of R and Rf independently represents H or C1-6alkyl.

Advantageously, R⁵ may represent a C1-20 alkyl moiety, for example a C1-6alkyl, for example CH₂—, —(CH₂)₂—, —(CH₂)₃—, —(CH₂)₄—.

Advantageously, R⁶ represents an amino group, preferably —N(Rf)₂ wherein each occurrence of Rf independently represents H or C1-6alkyl, for example R⁶ may represent —NH₂.

Advantageously, the shape of the organosilica particles (filled particles or core/shell nanocapsules) may be tuned to obtain mostly particles of a specific shape (spherical, rice-shape, etc. . . . ) according to known methods. The particle shape may in turn have an effect on the mechanical properties of the hybrid hydrogel.

Advantageously, the molecule of interest may be selected from the group comprising proteins, enzymes, oligonucleotides, antibodies, peptides, PNA, DNA, RNA, gene fragments and small molecules with or without pharmaceutical or cosmetic activity.

Advantageously, the proteins may be fluorescence protein family such as GFP, RFP; Cytotoxic proteins such as: TRAIL/APO-2L, Onconase, Ricin, Parasporin; Therapeutic proteins: Insulin Family, Angiopoietin family, Coagulation factor proteins, Dystrophin, HIV antigen, Hepatitis C antigen.

Advantageously, the protein may be proteins for cosmetic for example Botulinum toxin protein family, Elastin, Collagen, Keratin, Calcitonin, Silk proteins.

Advantageously, the enzymes may be RNAase, Hyaluronidase, Lysosomal enzyme acid alpha-glucosidase, Galactosidase, Glucocerebrosidase, Streptokinase, Urokinase, Altepase, Thymidine kinase, cytosine deaminase.

Advantageously, the oligonucleotides may be DNA (Deoxyribonucleic acid), RNA (Ribo Nucleic acid), PNA (Peptide Nucleic acid), LNA (Locked Nucleic Acid).

Advantageously, the antibodies may be selected from the group comprising Trastuzumab, Bevacizumab, Cetuximab, Mylotarg, Alemtuzumab, Rituximab, Brentuximab.

Advantageously, the small molecules with or without pharmaceutical activity may be for example sugars and/or polypeptide.

Advantageously, the nanoencapsulated biomolecule may be selected from the group comprising proteins, enzymes, oligonucleotides, antibodies, peptides, PNA, DNA, RNA, and gene fragments.

Advantageously, the cleavage/degradation of the linker *-R¹-L₁-R²-* and/or #-R³-L₂-R⁴-# may be independently triggered by any suitable means. For example, it may be a change in pH (either an increase or a decrease), a change in redox potential, the presence of reduction or oxidation agent, application of UV, visible, infrared or near infrared light, ultrasounds, electromagnetic radiation, a change in temperature, enzymatic cleavage, DNA binding, etc. . . . . The following Table 1 gives examples of cleavage/degradation triggering means for each of the aforementioned types of responsively cleavable linkers:

TABLE 1

| | Exemplary |
|---|---|
| Cleavable bond or linker | Triggers |
| Disulfide | Reducing |
| | agents (e.g., |
| | NaBH₄, |
| | dithiothreitol |
| | (DTT), |
| | glutathione |
| | (GSH)) |
| Diselenide | Reducing |
| | agents (e.g. |
| | thiols, metal |
| | complexes) |
| Ester | pH, |
| | enzymatic |
| | cleavage |
| | (e.g. |
| | esterase) [8] |
| Amide | Enzymatic |
| | cleavage |
| | (e.g. |
| | amidase) [9] |
| Imine | pH |
| Acetal/ketal/thioketal | pH |
| Anhydride | pH |
| Urea/thiourea | Enzymatic |
| | cleavage |
| | (e.g. urease) |
| | [10] |
| Hydrazone | pH |
| Oxyme | pH |
| Boronic acid (complexed with diols) | pH, sugars |
| Boronic esters | pH, reducing |
| | agents (e.g., |
| | LiAlH₄) |
| 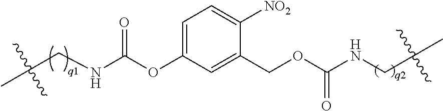 | Light |
| 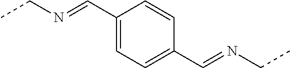 | pH |
| 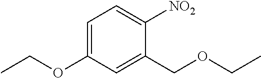 [SMILES: CCOCc1cc(OCC)ccc1[N+](=O)[O-]] | Light |
| 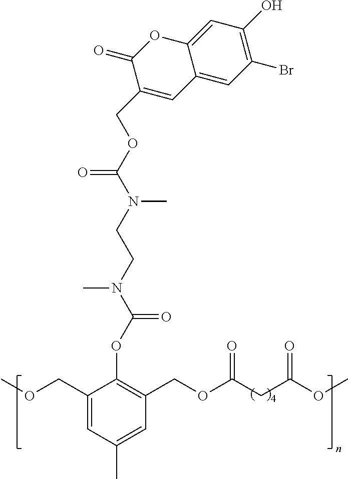 | Infrared |

L₁ and L₂ may be the same or different. When they are different (especially when the type of cleavable bond(s) in the linkers is different and/or are cleaved with a different stimulus), the degradation of the first hydrogel network may be controlled/effected independently from the degradation of the organosilica material (degradable nanoparticles or core/shell nanocapsules) that may be covalently bound to the first hydrogel framework.

In yet another aspect, there is provided a method for producing a new class of hybrid hydrogel materials.

This new class of materials includes polymer framework systems whose framework is formed from precursors having one of the following structures:
a monomer precursor of formula (IV)

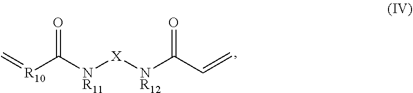

at least one bivalent molecular crosslinker precursor having the structure A-R¹-L₁-R²-A,
wherein each occurrence of A independently represents a hydrolysable or nonhydrolyzable group, provided that at least one occurrence of A represents a hydrolysable group, wherein (i) when A represents a nonhydrolyzable group, it may be selected from the group comprising an optionally substituted C1-20alkyl, C2-20alkenyl or C2-20alkynyl moiety, an optionally substituted C1-20heteroalkyl, C2-20heteroalkynyl or C2-20heteroalkynyl moiety, or an optionally substituted phenyl moiety (substituents on the aforementioned phenyl, alkyl, alkenyl, alkynyl, heteroalkyl, heteroalkenyl and heteroalkynyl moieties may be independently selected from the group comprising halogen, —NO₂, —CN, isocyano, C1-6alkoxy, an oxirane/epoxyde moiety, —N(R)₂ wherein each occurrence of R is independently selected from the group comprising H or C1-6alkyl); and (ii) when A represents a hydrolysable group, it may be selected from the group comprising a C1-6alkoxy, C1-6acyloxy, halogen or amino moiety;
L₁ independently represents a stable or responsively cleavable covalent bond; and
R¹ and R² independently represent an optionally substituted C1-20alkylenyl moiety, an optionally substituted C1-20heteroalkylenyl moiety, an optionally substituted ethylenyl moiety, —C≡C— or an optionally substituted phenyl moiety (i.e., a moiety comprising a phenyl group that may have on one or both sides an alkylenyl or heteroalkylenyl group: in other words “optionally substituted phenyl moiety” encompasses moieties such as —C₀₋₁₀alkyl-Ph-, —C₀₋₁₀heteroalkyl-Ph-, —C₀₋₁₀alkyl-Ph-C₀₋₁₀alkyl-, —C₀₋₁₀alkyl-Ph-C₀₋₁₀heteroalkyl-, —C₀₋₁₀heteroalkyl-Ph-C₀₋₁₀alkyl-, —C₀₋₁₀heteroalkyl-Ph-C₀₋₁₀heteroalkyl-, wherein Ph may bear one or more additional substituents, independently, such as halogen, C1-6alkyl, —NO₂, —CN, isocyano, —ORp, —N(Rp)₂ as defined below); The aforementioned C1-20alkylenyl, C1-20heteroalkylenyl or ethylenyl moiety may bear one or more substituents such as halogen or —OR where R may represent H or C1-6alkyl; The aforementioned phenyl moiety may bear one or more substituents, independently, such as halogen, C1-6alkyl, —NO₂, —CN, isocyano, —OR^{p}, —N(R^{p})₂ wherein each occurrence of R^{p} independently represents H or C1-6alkyl,
R₁₀ independently represents an optionally substituted C1-20 alkylenyl moiety,
R₁₁ and R₁₂ independently represent H, an optionally substituted C1-20 alkyl moiety, an optionally substituted C1-20 alkylenyl moiety, an optionally substituted C1-20heteroalkylenyl moiety, an optionally substituted ethylenyl moiety, —C≡C— or an optionally substituted phenyl moiety (i.e., a moiety comprising a phenyl group that may have on one or both sides an alkyl or heteroalkyl branch: in other words “optionally substituted phenyl moiety” encompasses moieties such as C₀₋₁₀alkyl-Ph, C₀₋₁₀heteroalkyl-Ph, C₀₋₁₀alkyl-Ph-C₀₋₁₀alkyl, C₀₋₁₀alkyl-Ph-C₀₋₁₀heteroalkyl, C₀₋₁₀heteroalkyl-Ph-C₀₋₁₀alkyl, C₀₋₁₀heteroalkyl-Ph-C₀₋₁₀heteroalkyl, wherein Ph may bear one or more additional substituents, independently, such as halogen, C1-6alkyl, —NO₂, —CN, isocyano, —ORp, —N(Rp)₂ as defined below); The aforementioned C1-20alkylenyl, C1-20heteroalkylenyl or ethylenyl moiety may bear one or more substituents, independently, such as halogen or —OR where R may represent H or C1-6alkyl; The aforementioned phenyl moiety may bear one or more substituents, independently, such as halogen, C1-6alkyl, —NO₂, —CN, isocyano, —OR^{p}, —N(R^{p})₂ wherein each occurrence of R^{p} independently represents H or C1-6alkyl, X independently represents an optionally substituted C1-20 alkyl moiety.

Advantageously, in the molecular crosslinker precursor A-R¹-L₁-R²-A, each occurrence of A may independently represent a nucleophilic moiety, preferably one that can undergo a Michael-type nucleophilic addition onto the double bond of the monomer precursor (IV). For example, each occurrence of A may independently represent —N(Rf)₂ wherein each occurrence of Rf may represent H or C1-6alkyl.

Advantageously, L1, R1, R2, R10, R11, R12 and X are independently as defined generally and in any variants above.

Thus, in one aspect, there is provided a method of preparing a hydrogel by covalently introducing a preselected precursor (general structure: monomer precursor of formula (IV)) with a molecular crosslinker precursor (general structure: A-R¹-L₁-R²-A) as defined herein, in the framework of the hydrogel material itself.

As such, the hydrogels present controlled self-destructive behavior in the environment where it is intended to perform its activity. The controlled self-destructive behavior is a property that provides numerous avenues of important applications for such hydrogel, ranging from medical to cosmetics.

The practitioner has a well-established literature of polymer and/or hyrdogel materials chemistry to draw upon, in combination with the information contained herein, for guidance on synthetic strategies, protecting groups, and other materials and methods useful for the synthesis of the disintegrable materials of this invention.

Advantageously, the method may comprise steps of:
a) dissolving in water or alcoholic solutions:
   a monomer precursor of formula (IV)

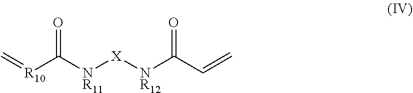

at least one bivalent molecular crosslinker precursor having the structure A-R¹-L₁-R²-A,
Optionally, a nanoencapsulated bioactive macromolecule or bioactive macromolecule cluster, and
Optionally, a selected precursor of formula B—R⁸
b) Stirring the solution obtained in step a), at any appropriate temperature, thereby allowing the polymerization carried out to form the hydrogel (first hydrogel),
c) Optionally, adding a suitable second polymer solution, for example alginate solution, which may be added concomitantly with step a) or separately from step a);
d) Optionally adding a suitable organic solvent, thereby precipitating the hydrogel:
wherein:
each occurrence of A or B independently represents a hydrolysable or nonhydrolyzable group, provided that at least one occurrence of A represents a hydrolysable group, wherein (i) when A or B independently represents a nonhydrolyzable group, it may be selected from the group comprising an optionally substituted C1-20alkyl, C2-20alkenyl or C2-20alkynyl moiety, an optionally substituted C1-20heteroalkyl, C2-20heteroalkynyl or C2-20heteroalkynyl moiety, or an optionally substituted phenyl moiety; wherein the substituents on the phenyl, alkyl, alkenyl, alkynyl, heteroalkyl, heteroalkenyl and heteroalkynyl moieties may be independently selected from the group comprising halogen, —NO₂, —CN, isocyano, C1-6alkoxy, an oxirane/epoxyde moiety, —N(R)₂ wherein each occurrence of R is independently selected from the group comprising H or C1-6alkyl; and (ii) when A or B independently represents a hydrolysable group, it may be selected from the group comprising a C1-6alkoxy, C1-6acyloxy, halogen or amino moiety;
L₁ independently represents a responsively cleavable covalent bond, a moiety containing a responsively cleavable covalent bond or a stable covalent bond; and
R¹ and R² independently represent an optionally substituted C1-20 alkylenyl moiety, an optionally substituted C1-20heteroalkylenyl moiety, an optionally substituted ethenylenyl moiety, —C≡C— or an optionally substituted phenyl moiety (i.e., a moiety comprising a phenyl group that may have on one or both sides an alkylenyl or heteroalkylenyl group: in other words “optionally substituted phenyl moiety” encompasses moieties such as —C₀₋₁₀alkyl-Ph-, —C₀₋₁₀heteroalkyl-Ph-, —C₀₋₁₀alkyl-Ph-C₀₋₁₀alkyl-, —C₀₋₁₀alkyl-Ph-C₀₋₁₀heteroalkyl-, —C₀₋₁₀heteroalkyl-Ph-C₀₋₁₀alkyl-, —C₀₋₁₀ heteroalkyl-Ph-C₀₋₁₀heteroalkyl-, wherein Ph may bear one or more additional substituents, independently, such as halogen, C1-6alkyl, —NO₂, —CN, isocyano, —ORp, —N(Rp)₂ as defined below); The aforementioned C1-20 alkylenyl, C1-20 heteroalkylenyl or ethenylenyl moiety may bear one or more substituents, independently, such as halogen or —OR where R may represent H or C1-6 alkyl; The aforementioned phenyl moiety may bear one or more substituents, independently, such as halogen, C1-6alkyl, —NO₂, —CN, isocyano, —ORp, —N(Rp)2 wherein each occurrence of Rp independently represents H or C1-6alkyl; wherein *-R¹-L₁-R²-* may independently comprise a sugar derivative such as mannose, a hyaluronic acid derivative, collagene, an aminoacid or a peptide moiety;
R₁₀ independently represents an optionally substituted C1-20 alkylenyl moiety, wherein the C1-20 alkylenyl moiety may bear one or more substituents, independently, such as halogen or —OR where R may represent H or C1-6alkyl;
R₁₁ and R₁₂ independently represent an optionally substituted C1-20 alkyl, C1-20alkenyl or C1-20alkynyl moiety, an optionally substituted C1-20heteroalkyl moiety, or an optionally substituted phenyl moiety (i.e., a moiety comprising a phenyl group that may have on one or both sides an alkyl or heteroalkyl branch: in other words “optionally substituted phenyl moiety” encompasses moieties such as C₀₋₁₀alkyl-Ph, C₀₋₁₀heteroalkyl-Ph, C₀₋₁₀alkyl-Ph-C₀₋₁₀alkyl, C₀₋₁₀alkyl-Ph-C₀₋₁₀heteroalkyl, C₀₋₁₀heteroalkyl-Ph-C₀₋₁₀alkyl, C₀₋₁₀heteroalkyl-Ph-C₀₋₁₀heteroalkyl, wherein Ph may bear one or more additional substituents, independently, such as halogen, C1-6alkyl, —NO₂, —CN, isocyano, —ORp, —N(Rp)₂ as defined below); Each of the foregoing C1-20 alkyl, C1-20alkenyl, C1-20alkynyl or C1-20heteroalkyl moiety may bear one or more substituents, independently, such as halogen or —OR where R may represent H or C1-6alkyl; The phenyl moiety may bear one or more substituents, independently, such as halogen, C1-6alkyl, —NO₂, —CN, isocyano, —ORp, —N(Rp)2 wherein each occurrence of Rp independently represents H or C1-6alkyl;
X independently represents an optionally substituted C1-20 alkylenyl moiety, wherein the C1-20 alkylenyl moiety may bear one or more substituents, independently, such as halogen or —OR where R may represent H or C1-6alkyl.

Advantageously, in the molecular crosslinker precursor A-R¹-L₁-R²-A, each occurrence of A may independently represent a nucleophilic moiety, preferably one that can undergo a Michael-type nucleophilic addition onto the double bond of the monomer precursor (IV). For example, each occurrence of A may independently represent —N(Rf)₂ wherein each occurrence of Rf may represent H or C1-6alkyl.

Advantageously, at least two different molecular crosslinker precursors A-R¹-L₁-R²-A are used, wherein in one molecular crosslinker precursor L₁ represents a responsively cleavable covalent bond or a moiety containing a responsively cleavable covalent bond as described generally and in any variant herein, and in the other L₁ represents a stable covalent bond.

Advantageously, R₁, R₂, R₁₀, R₁₁, R₁₂, L₁, and X may be as described generally and in any variant above, and in any combination.

Advantageously, the monomer precursor may be of formula (IVa)

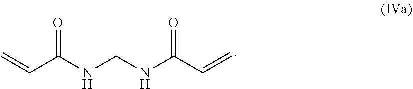

Advantageously, the amount and/or concentration of monomer precursor dissolved in solution of step a) may range anywhere from 0.1% to 100% w/v. For example, it may be from 2% to 30% w/v, for example from 4% to 30% w/v, preferably from 9% to 18% w/v.

Advantageously, the molecular crosslinker precursor A-R¹-L₁-R²-A may be a precursor selected from the group comprising:

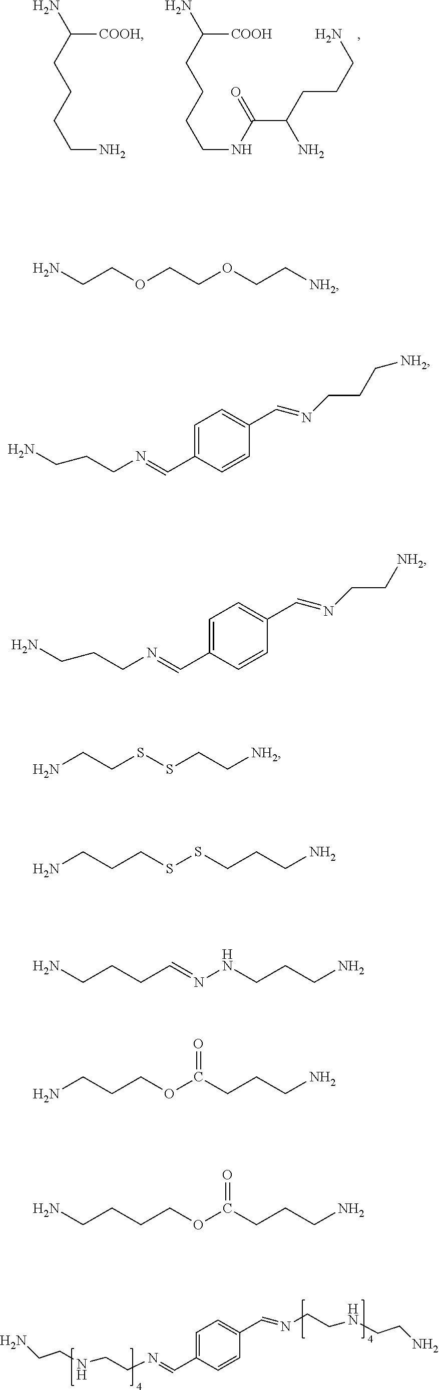

Advantageously, the amount and/or concentration of molecular crosslinker precursor dissolved in solution of step a) may range anywhere from 0.1% to 100% w/v. For example, it may be from 0.5% to 20% w/v, for example from 1% to 20% w/v, preferably from 2% to 8% w/v.

Advantageously, when the process comprises in step a selected precursor of formula B—R⁸, B may independently represent a hydrolysable or nonhydrolyzable group, wherein (i) when B represents a nonhydrolyzable group, it may be selected from the group comprising an optionally substituted C1-20alkyl, C2-20alkenyl or C2-20alkynyl moiety, an optionally substituted C1-20heteroalkyl, C2-20heteroalkynyl or C2-20heteroalkynyl moiety, or an optionally substituted phenyl moiety; wherein the substituents on the phenyl, alkyl, alkenyl, alkynyl, heteroalkyl, heteroalkenyl and heteroalkynyl moieties may be independently selected from the group comprising halogen, —NO₂, —OH, —CN, isocyano, C1-6alkoxy, an oxirane/epoxyde moiety, —N(R)₂ wherein each occurrence of R is independently selected from the group comprising H or C1-6alkyl; and (ii) when B represents a hydrolysable group, it may be selected from the group comprising a C1-6alkoxy, C1-6acyloxy, halogen or amino moiety. Preferably B represents N(R)₂ wherein each occurrence of R is independently selected from the group comprising H or C1-6alkyl.

Advantageously, in a selected precursor of formula B—R⁸, B may independently represent a nucleophilic moiety, preferably one that can undergo a Michael-type nucleophilic addition onto the double bond of the monomer precursor (IV). For example, each occurrence of A may independently represent —N(Rf)₂ wherein each occurrence of Rf may represent H or C1-6alkyl. Advantageously, R⁸ may be as described generally and in any variant above. For example,
R⁸ may represent an optionally substituted C1-20 alkyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety (i.e., a moiety comprising a phenyl group that may have on one or both sides an alkyl or heteroalkyl branch: in other words “optionally substituted phenyl moiety” encompasses moieties such as C₀₋₁₀alkyl-Ph, C₀₋₁₀heteroalkyl-Ph, C₀₋₁₀alkyl-Ph-C₀₋₁₀alkyl, C₀₋₁₀alkyl-Ph-C₀₋₁₀heteroalkyl, C₀₋₁₀heteroalkyl-Ph-C₀₋₁₀alkyl, C₀₋₁₀heteroalkyl-Ph-C₀₋₁₀heteroalkyl, wherein Ph may bear one or more additional substituents, independently, such as halogen, C1-6alkyl, —NO₂, —CN, isocyano, —ORp, —N(Rp)₂ as defined below); Each of the foregoing C1-20 alkyl, C1-20heteroalkyl or C1-20alkylphenyl moieties may bear one or more substituents, independently, such as halogen, —OR, —CO₂R or —N(Rp)2 where R may represent H or C1-6alkyl, and each occurrence of Rp may independently represent H or C1-6alkyl; The phenyl moiety may bear one or more substituents, independently, such as halogen, C1-6alkyl, —NO2, —CN, isocyano, —ORp, —N(Rp)2 wherein each occurrence of Rp independently represents H, C1-6alkyl or C1-6 alkoxy;
R⁸ may represent the residue of the corresponding amino acid H₂NR⁸;
R⁸ may represent a C1-C6 alkyl substituted with a carboxyl moiety, a C1-C6 alkyl substituted with one or more hydroxyl groups, C1-C6 alkoxy, C1-C6 alkyl substituted with —N(Rp)2 wherein each occurrence of Rp independently represents a C1-6alkyl;
R⁸ may represent a C1-C6 alkyl substituted with —N(Rp)2 wherein each occurrence of Rp independently represents a C1-6alkyl; for example a C1-C2 alkyl substituted with-N(Rp)2 wherein each occurrence of Rp independently represents a C1-2alkyl. R⁸ may represent a C2 alkyl substituted with-N(Rp)2 wherein each occurrence of Rp independently represents a C1 alkyl. For example R⁸ mar represent —(CH₂)NMe₂;
R⁸ may represent a C2 alkyl substituted with-N(Rp)2 wherein each occurrence of Rp independently represents a C1 alkyl. For example R⁸ mar represent —(CH₂)NMe₂;
R⁸ may represent independently from other occurrences of R8 a C1-20alkylphenyl moiety optionally substituted with one or more —OR wherein R may represent H or C1-6alkyl. For example, R⁸ may represent independently from other occurrences of R8 a C1-6alkylphenyl moiety optionally substituted with one or more —OR wherein R may represent H or C1-6alkyl. For example, R⁸ may represent independently from other occurrences of R8 a C1-6alkyl moiety bearing a catechol moiety;
R⁸ may be independently a group of following formula:

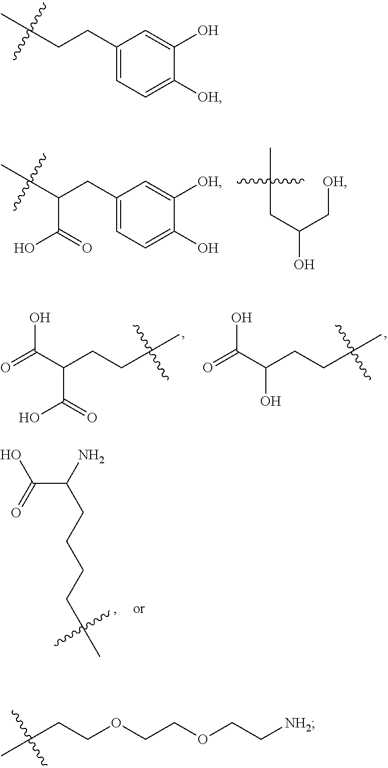

R⁸ may be independently a hyaluronic acid, alginic acid, peptide, cellulose, amino acid, sugar (for example glucose, lactose or mannose derivatives), or oligonucleotide moiety;
R⁸ may be independently a C1-20alkyl or C1-20heteroalkyl moiety, preferably C1-6alkyl or C1-6heteroalkyl, most preferably C1-6alkyl, bearing an organosilica core/shell nanocapsule, preferably the organosilica matrix may be porous, most preferably mesoporous. Preferably, the organosilica core/shell nanocapsule may be degradable/disintegrable in that its shell framework contains Si adjacent sites covalently bound via a responsively cleavable linker, as described in WO 2015/189402. Advantageously, the organosilica core/shell nanocapsule may encapsulate a bioactive macromolecule or bioactive macromolecule cluster, and/or another molecule of interest that may or may not have biological activity and/or pharmaceutical or cosmetic activity.

Advantageously, when the process comprises in step a) a selected precursor of general formula B—R⁸, it may be selected from the group comprising:

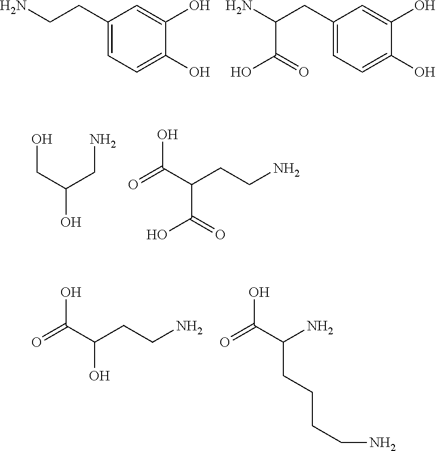

Advantageously, the amount and/or concentration of precursor (general formula B—R⁸) dissolved in solution of step a) may range anywhere from 0.1% to 100% w/v. For example, it may be from 1% to 10% w/v, preferably from 1% to 5% w/v.

Advantageously, when the process comprises in step a) the addition of nanoencapsulated molecules or bioactive macromolecules or biomacromolecule cluster, it advantageously allows to prepare hydrogels comprising nanoencapsulated molecules or bioactive macromolecules or biomacromolecule cluster.

Advantageously, the nanoencapsulated molecules or bioactive macromolecules or biomacromolecule cluster is as mentioned above, generally and in any variant described above, and in any combination.

Advantageously, the shell of said nanocapsule may be made of hybrid organosilica material comprising a three-dimensional framework of Si—O bonds, wherein at least a subset of Si atoms in the material's framework are connected to at least another Si atom in the framework through a linker having the following structure:

#-R³-L₂-R⁴-#;

wherein:
each occurrence of # denotes a point of attachment to a Si atom in the hybrid organosilica material's framework;
L₂ independently represents a responsively cleavable covalent bond or a stable bridging ligand; preferably a responsively cleavable covalent bond; and
R³ and R⁴ independently represent an optionally substituted C1-20 alkylenyl moiety, an optionally substituted C1-20 heteroalkylenyl moiety, an optionally substituted ethenylenyl moiety, —C≡C— or an optionally substituted phenyl moiety (i.e., a moiety comprising a phenyl group that may have on one or both sides an alkylenyl or heteroalkylenyl group: in other words “optionally substituted phenyl moiety” encompasses moieties such as —C₀₋₁₀alkyl-Ph-, —C₀₋₁₀heteroalkyl-Ph-, —C₀₋₁₀alkyl-Ph-C₀₋₁₀alkyl-, —C₀₋₁₀alkyl-Ph-C₀₋₁₀heteroalkyl-, —C₀₋₁₀heteroalkyl-Ph-C₀₋₁₀alkyl-, —C₀₋₁₀heteroalkyl-Ph-C₀₋₁₀heteroalkyl-, wherein Ph may bear one or more additional substituents, independently, such as halogen, C1-6alkyl, —NO₂, —CN, isocyano, —ORp, —N(Rp)₂ as defined below); The aforementioned C1-20alkylenyl, C1-20 heteroalkylenyl or ethenylenyl moiety may bear one or more substituents, independently, such as halogen or —OR where R may represent H or C1-6alkyl; The aforementioned phenyl moiety may bear one or more substituents, independently, such as halogen, C1-6alkyl, —NO₂, —CN, isocyano, —OR^{p}, —N(R^{p})₂ wherein each occurrence of R^{p} independently represents H or C1-6alkyl,
and
the nanocapsule outer surface may comprise a group of formula

#-R⁵R⁶
wherein
each occurrence of # denotes a point of attachment to a Si atom in the hybrid organosilica material's framework;
R⁵ independently represents an optionally substituted C1-20alkylenyl moiety, an optionally substituted C1-20heteroalkylenyl moiety, an optionally substituted ethenylenyl moiety, —C≡C— or an optionally substituted phenyl moiety (i.e., a moiety comprising a phenyl group that may have on one or both sides an alkylenyl or heteroalkylenyl group: in other words “optionally substituted phenyl moiety” encompasses moieties such as —C₀₋₁₀alkyl-Ph-, —C₀₋₁₀heteroalkyl-Ph-, —C₀₋₁₀alkyl-Ph-C₀₋₁₀alkyl-, —C₀₋₁₀alkyl-Ph-C₀₋₁₀heteroalkyl-, —C₀₋₁₀heteroalkyl-Ph-C₀₋₁₀alkyl-, —C₀₋₁₀heteroalkyl-Ph-C₀₋₁₀heteroalkyl-, wherein Ph may bear one or more additional substituents, independently, such as halogen, C1-6alkyl, —NO₂, —CN, isocyano, —ORp, —N(Rp)₂ as defined below); The aforementioned C1-20alkylenyl, C1-20heteroalkylenyl or ethenylenyl moiety may bear one or more substituents, independently, such as halogen or —OR where R may represent H or C1-6alkyl; The aforementioned phenyl moiety may bear one or more substituents, independently, such as halogen, C1-6alkyl, —NO2, —CN, isocyano, —ORp, —N(Rp)2 wherein each occurrence of Rp independently represents H or C1-6alkyl;

R⁶ independently represents —OR, —SR or —N(Rf)₂; preferably —N(Rf)₂; wherein each occurrence of R and Rf independently represents H or C1-6alkyl.

Advantageously, #-R³-L₂-R⁴-# may be as defined generally and in any variant above. For example, #-R³-L₂-R⁴-# may represent independently a responsively cleavable moiety such as:

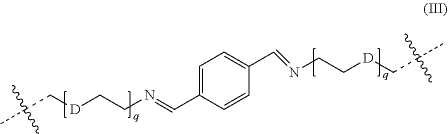

wherein q and D are as as defined generally and in any variant above;

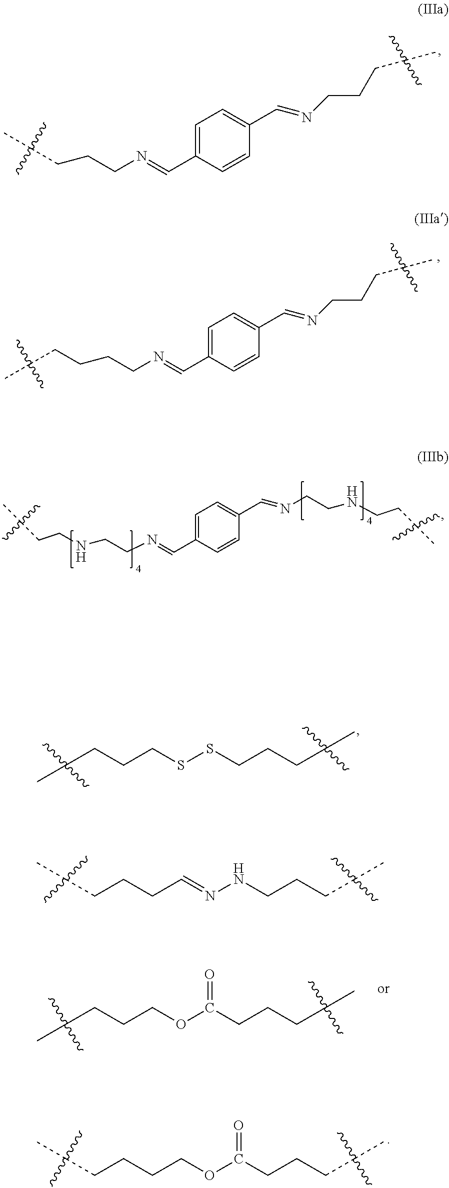

#-R³-L₂-R⁴-# may be introduced in the hybrid organosilica framework via a precursor

(Z)₃Si—R³-L2-R⁴—Si(Z)₃;

wherein L₂, R³, and R⁴ are as defined generally and any variant above, which is chemically inserted within the framework of the hybrid organosilica matrix via sol-gel chemistry. In the above, Z may independently represent a hydrolysable or nonhydrolyzable group, provided that on each occurrence of Si, at least one occurrence of Z represents a hydrolysable group.

When occurrences of Z represent a hydrolysable group, it may be selected from the group comprising a C1-6 alkoxy, C1-6 acyloxy, halogen or amino moiety. Advantageously, when occurrences of Z represent a hydrolysable group, Z may represent Cl, -OMe, -OEt, -OiPr or -OtBu.

When occurrences of Z represent a nonhydrolyzable group, they may independently be selected from the group comprising an optionally substituted C1-20 alkyl, C2-20 alkenyl or C2-20 alkynyl moiety, an optionally substituted C1-20 heteroalkyl, C2-20 heteroalkynyl or C2-20 heteroalkynyl moiety, or an optionally substituted phenyl moiety, wherein the substituents on the phenyl, alkyl, alkenyl, alkynyl, heteroalkyl, heteroalkenyl and heteroalkynyl moieties may be independently selected from the group comprising halogen, —NO₂, —CN, isocyano, C1-6 alkoxy, an oxirane/epoxyde moiety, —N(R)₂ wherein each occurrence of R is independently selected from the group comprising H or C1-6 alkyl.

Advantageously, when occurrences of Z represent a nonhydrolyzable group, Z may represent C1-6 alkyl or C2-6 alkenyl; preferably -Me, -Et or —CH═CH₂; most preferably -Me or -Et.

The insertion of the responsively cleavable linker #-R³-L₂-R⁴-# within the framework of the hybrid organosilica matrix may be performed during the synthesis of the hybrid organosilica material itself, no additional step is required, if not the preparation of the required (Z)₃Si—R³-L₂-R⁴—Si(Z)₃ precursor, which may also be carried out in situ.

Advantageously, the following may be used as (Z)₃Si—R³-L₂-R⁴—Si(Z)₃ precursor:

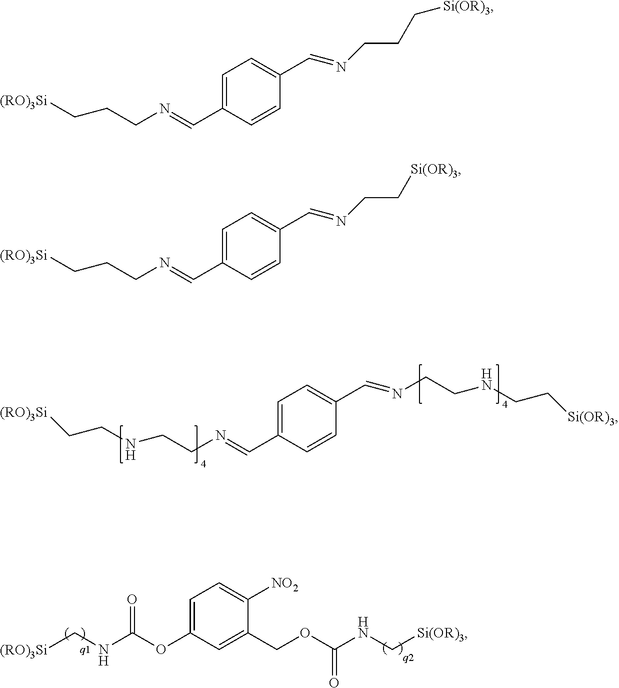

wherein q and D are as as defined generally and in any variant above;

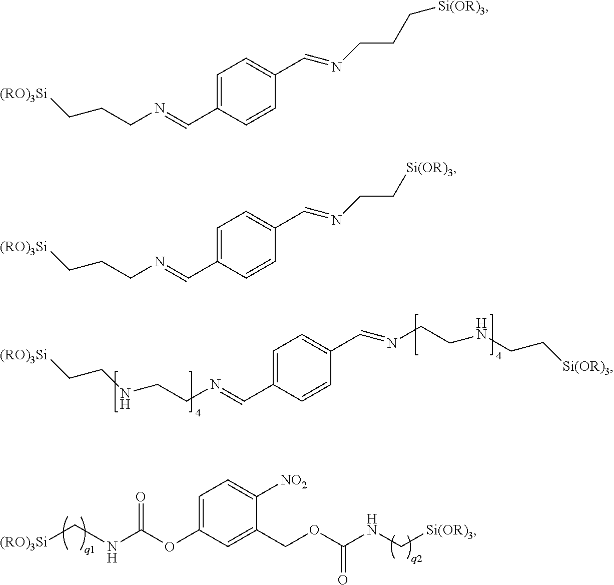

wherein q1 and q2 are as as defined generally and in any variant above;

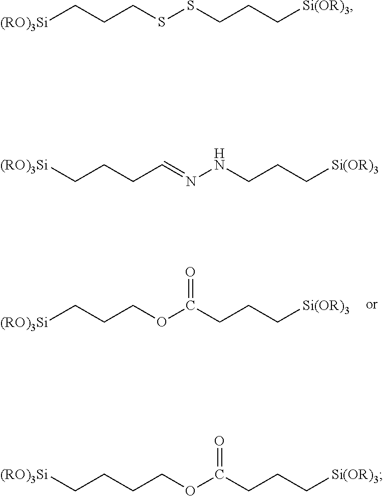

wherein each occurrence of R may independently represent Me, Et, iPr or tBu.

Advantageously, R⁵ independently represents a C1-20 alkyl moiety, for example a C1-6alkyl, for example CH₂—, —(CH₂)₂—, —(CH₂)₃—, —(CH₂)₄—.

Advantageously, R⁶ independently represents N, preferably —N(Rf)2 wherein each occurrence of Rf independently represents H or C1-6alkyl, for example —NH₂.

Advantageously, when the outer surface of the nanocapsule comprises a group of formula #-R⁵R⁶, it improves the attachment of the nanocapsule to the first hydrogel framework. In particular, as illustrated herein, when a group of formula #-R⁵R⁶ as defined herein comprises an amino group, it allows to covalently link the nanocapsule to the first hydrogel framework.

Functionalized organosilane chemistry is well known, and the reader may refer to the following citations for illustrative synthetic guidance that may be readily adapted in the context of the present invention. [2]

Advantageously, the amount of organosilica (nano)particles or core/shell nanocapsules with the encapsulated active molecules can vary between 0.1 and 20% w/v (weight of the silica nanoparticles vs volume of the pre-hydrogel). For example, it may be 1%, 2%, 5%, 0.1% depending on the elasticity and delivery that is desired.

Advantageously, the process may further comprise in step a) or after step a) the addition of a solution of alginate, for example an aqueously solution of sodium alginate. Advantageously, gelation of the hybrid hydrogel may be effected by addition of an alkali earth metal salt aqueous solution, such as a calcium salt solution (e.g., calcium chloride).

Advantageously, the alginate may be any alginate known to the person of ordinary skill in the art. The alginate may be as defined generally or in any variant above. For example, the alginate may have formula (UU) as defined herein.

Advantageously, the solution of alginate may be any suitable solution known to the skilled artisan. The amount and/or concentration of alginate in solution may range anywhere from 0.1% to 100% w/v. For example, it may be an aqueous solution (water or hydroalcoholic, preferably water) with concentration from 0.01% to 5% w/v.

Advantageously, when the process further comprises the addition of a solution of alginate in step a) or after step a), it allows to prepare a hybrid hydrogel.

Advantageously, the amount and/or concentration of alginate added in solution of step a) or after step a) may be from 0 to 50% w/v, for example 0 to 40% w/v, for example 0 to 30% w/v, for example 0 to 20% w/v, for example 0 to 10%, preferably from 0.01 to 5% w/v.

Advantageously, the stirring in step b) may be carried out at any suitable temperature with any suitable process and/or device known to the person of ordinary skill in the art.

Advantageously, a pH adjusting agent may be used to modulate the pH to the desired value, for example in step b). The pH of the solution may be adjusted using any suitable technique. As the pH-adjusting agent, there can be mentioned, for example, acids such as sulfuric acid, hydrochloric acid and the like; and alkalis such as sodium hydroxide, ammonia and the like. Advantageously, when disintegrable hybrid organosilica nanoparticles or core/shell nanocapsules nanocapsules are used, the pH of the reaction system may be preferably adjusted to >7, for example 7.5-10, more preferably 8-9, most preferably about 8.

Advantageously, the organic solvent in step c) may be any suitable organic solvent known to the person of ordinary skill in the art. It may be for example an organic solvent selected from the group comprising methanol, ethanol, n-propanol and/or any other protic solvent, or mixture of two or more thereof.

Advantageously, the hybrid hydrogel comprising or not the nanoencapsulated bioactive macromolecule or bioactive macromolecule cluster and/or another molecule of interest that may or may not have biological activity and/or pharmaceutical or cosmetic activity, obtained with the process of the invention may be transparent.

Advantageously, the hybrid hydrogel comprising or not the nanoencapsulated bioactive macromolecule or bioactive macromolecule cluster and/or another molecule of interest that may or may not have biological activity and/or pharmaceutical or cosmetic activity, may be obtained at room temperature, for example between 20 to 35° C., in an aqueous solvent.

Advantageously, the hybrid hydrogel of the invention may be obtained according to a catalyst-free Michael -type addition.

Advantageously, the hybrid hydrogel may be formed in-situ and does not need any external agent and/or supplemental agent for the reticulation/crosslinking process.

Advantageously, the hybrid hydrogel may be formed in-situ under physiological condition.

Another object of the present invention is a hydrogel obtainable by a method of the invention.

Another object of the present invention is a hybrid hydrogel obtainable by the method of the invention.

Hybrid hydrogels described herein are useful for any medical application where it is desirable to fill a hole, for example a lesion, a wound, etc.

Hybrid hydrogels described herein are also useful for any application in the gastrointestinal field where it is desirable to fill a vessel, a tract, a hole, or an opening, to prevent, stop or alleviate/lessen/relieve the effects of physiological leakages, and anywhere a wound can be healed.

For example, hybrid hydrogels described herein are useful for any application where it is desirable to fill a vessel, such as in the treatment of fistulas, in particular gastrointestinal fistulas (by occluding the fistula tract).

Hybrid hydrogels described herein are also useful for the treatment or prevention of gastro-esophageal reflux disease (GERD) by restoring the lower esophageal sphincter pressure.

Hybrid hydrogels described herein are also useful for the treatment or prevention of insulin-resistance/metabolic syndrome, by creating a physical barrier to the absorption of nutrients in crucial segments of the small bowel.

Other applications in the GI field include treatment or prevention of bowel and urinary incontinence by sphincter augmentation, treatment or prevention of anastomotic leaks by intraluminal chemical sealing, treatment or prevention of SEMS (self expandable metal stents) migration, regenerative application for tissue regeneration.

Hybrid hydrogels described herein as mentioned above are also useful for any application where controlled release of a molecule of interest, bioactive molecule or biomolecule cluster is desired.

Hybrid hydrogels described herein are also particularly adapted for uses of this type of materials where the self-destructive behavior that characterizes the core/shell silica nanocapsules and the hybrid hydrogels of the invention provides an advantage, and for applications where preservation of the biological activity of the biomacromolecule is needed.

In particular, in contrast to conventional hydrogel materials known in the art, the hybrid hydrogels described herein have the unexpected property of being formed in-situ without any external stimuli.

In addition, in contrast to conventional hydrogel materials known in the art, the hybrid hydrogels described herein allow to provide a physical support, notably for in vivo medical applications, and also be biodegradable.

Moreover, hybrid hydrogels described herein may completely lose their structural integrity (disintegration) upon application of a suitable stimuli and/or under the biological activity of proteins, for example enzymes. In other words, for biomedical applications, for example application onto tissue and/or after injection, this means less bio-accumulation, better elimination, and less toxicity.

Owing to their disintegrable properties, hybrid hydrogel comprising core/shell silica nanocapsules prove much more efficient in releasing and delivering macromolecules that they encapsulate (e.g., therapeutically and/or cosmetically active macromolecular principles). In other words, release of the macromolecules trapped/encapsulated in the core/shell silica nanocapsules occurs much more efficiently. For biomedical applications (e.g., when the framework metal is Si), this means less bio-accumulation, better elimination, and less toxicity.

Accordingly, there is provided compositions comprising hybrid hydrogel described generally and in any variants herein and any compound and/or additive suitable for any one or more of the material's intended use describe above.

For example, there is provided a pharmaceutical composition comprising hybrid hydrogel described generally and in any variants herein, and a pharmaceutically acceptable carrier, adjuvant or vehicle. In exemplary embodiments, these compositions optionally further comprise one or more additional therapeutic agents.

The person of ordinary skill in the art, taking into consideration the common technical knowledge in the medical field, would know and/or select the additional therapeutic agents in light of the disease/condition to be treated.

In another example, there is provided a cosmetic composition comprising hybrid hydrogel described generally and in any variants herein, and a cosmetically acceptable carrier, adjuvant or vehicle. In certain embodiments, these compositions optionally further comprise one or more additional cosmetically useful agents.

In yet another example, there is provided a veterinary composition comprising hybrid hydrogel described generally and in any variants herein, and a pharmaceutically acceptable carrier, adjuvant or vehicle. In exemplary embodiments, these compositions optionally further comprise one or more additional therapeutic agents.

In another aspect, there is provided a hybrid hydrogel described generally and in any variants herein, for use as medicament.

In another aspect, there is provided a hybrid hydrogel described generally and in any variants herein, for use as medicament for sealing a wound, for enhancing tissue regeneration, fillers for example for submucosal fluid cushion for surgery, tissue reconstitution in a subject-in-need thereof.

In yet another aspect, there is provided a hybrid hydrogel described generally and in any variants herein, for use as medicament for treating diabetes or spinal cord injury.

In yet another aspect, there is provided a hybrid hydrogel described generally and in any variants herein, for use as medicament for treating hernia or ulcers.

In yet another aspect, there is provided a hybrid hydrogel described generally and in any variants herein, for use as medicament in cardiac repair.

In another aspect, there is provided a hybrid hydrogel described generally and in any variants herein, in a cosmetic composition.

In another aspect, there is provided a hybrid hydrogel described generally and in any variants herein, or a cosmetic composition described generally and in any variants herein, for delivering a cosmetically bioactive macromolecule and/or a cosmetically bioactive macromolecule to the skin.

The cosmetically bioactive macromolecule may be any cosmetically bioactive macromolecule and/or a cosmetically bioactive macromolecule known in the art. It may be, for example, selected from the group comprising collagen, keratin, elastin, calcitonin, hyaluronic acid, amino acids, retinol, antioxidants, vitamins or silk proteins.

In another aspect, there is provided a hybrid hydrogel described generally and in any variants herein for use as a medicament in the treatment of cancer, preferably tumors. Specifically, hybrid hydrogels described herein may be injected under a tumor to be excised, preferably a solid tumor, thereby allowing the resection of the tumor with minimal lesion to the surrounding tissue. The person of ordinary skill in the art, taking into consideration the common technical knowledge in the medical field, would know and/or select suitable therapeutic agents that may be used in association with the hybrid hydrogel for optimizing therapeutic success of the procedure. In particular, the person of ordinary skill in the art would select which therapeutic agent should be included into the hybrid hydrogel, for example in the pores and/or core of organosilica particles (plain nanoparticles or core/shell nanoparticles) that may be mixed in the hybrid hydrogel network and/or covalently conjugated to the first hydrogel network, as detailed supra. It may be for example any anti-cancerous drug or any suitable palliative drug appropriate for this type of surgical treatment (e.g., antiinflammatory) known from a person of ordinary skill in the art that could be linked and/or included into the hybrid hydrogel and/or encapsulated into the nanoparticles.

In another aspect, there is provided hybrid hydrogel described generally and in any variants herein, for delivering a cosmetically bioactive macromolecule to the skin. In exemplary embodiments, the cosmetically bioactive macromolecule may be collagen, keratin, elastin, calcitonin or silk proteins.

In another aspect, there is provided a method for systemically delivering a bioactive macromolecule, in a biologically active form, to a subject in need thereof, the method comprising, administering to the subject a therapeutically effective amount of a hybrid hydrogel described generally and in any variants herein. In exemplary embodiments, the bioactive macromolecule may be selected from the group comprising proteins, oligonucleotides, antibodies, peptides, PNA, DNA, RNA, gene fragments, a hormone, a growth factor, a protease, an extra-cellular matrix protein, an enzyme, an infectious viral protein, an antisense oligonucleotide, a dsRNA, a ribozyme, a DNAzyme, antibiotics, antinflammatory, steroids, chemiotherapeutics. In exemplary embodiments, the bioactive macromolecule may be an enzyme and said biological activity is a catalytic activity. In exemplary embodiments, the bioactive macromolecule may be a hormone and said biological activity is a ligand binding activity.

In another aspect, there is provided a unit dosage form for local delivery of a molecule to a tissue of a subject, the unit dosage form comprising, a therapeutically effective amount of a hybrid hydrogel described generally and in any variants herein or a pharmaceutical composition described generally and in any variants herein. In exemplary embodiments, the molecule may be selected from the group comprising proteins, oligonucleotides, antibodies, peptides, PNA, DNA, RNA, gene fragments, a hormone, a growth factor, a protease, an extra-cellular matrix protein, an enzyme, an infectious viral protein, an antisense oligonucleotide, a dsRNA, a ribozyme and a DNAzyme.

In another aspect, there is provided a method for treating a disease in a subject in need thereof, the method comprising administering to the subject a therapeutically effective amount of hybrid hydrogel described generally and in any variants herein, thereby treating the disease in the subject.

In another aspect, there is provided a delivery system for sealing a wound, for enhancing tissue regeneration, fillers for example for submucosal fluid cushion for surgery, tissue reconstitution in a subject-in-need thereof, said system comprising hybrid hydrogel described generally and in any variants herein.

In another aspect, there is provided a method of using hybrid hydrogel described generally and in any variants herein as controlled-release agents or carriers for macromolecular drug, protein, and vaccine delivery.

In another aspect, there is provided a method of using hybrid hydrogel described generally and in any variants herein for sealing acute and/or chronic wounds and/or perforation in a subject-in-need thereof, the method comprising administering to the subject a therapeutically effective amount of a hybrid hydrogel according to the invention or a pharmaceutical composition according to the invention, thereby sealing the wound and/or perforation.

In another aspect, there is provided a method for treating a disease, preferably cancer, most preferably cancer tumor, in a subject-in-need thereof, the method comprising administering to the subject a therapeutically effective amount of a hybrid hydrogel according to the invention or a pharmaceutical composition according to the invention, thereby treating the disease in the subject. Advantageously, hybrid hydrogels described herein may be injected under a tumor to be excised, preferably a solid tumor, in an amount sufficient to substantially detach/disengage the tumor from surrounding tissue, thereby allowing the resection of the tumor with minimal lesion to the surrounding tissue.

In another aspect, there is provided a method for treating diabetes, in a subject-in-need thereof, the method comprising administering to the subject a therapeutically effective amount of a hybrid hydrogel according to the invention or a pharmaceutical composition according to the invention, thereby treating the disease in the subject. The injected hybrid hydrogel may be advantageously loaded with insulin, for example in the pores and/or core of organosilica particles (plain nanoparticles or core/shell nanoparticles) that may be mixed in the hybrid hydrogel network and/or covalently conjugated to the first hydrogel network, as detailed supra, for sustained release of insulin.

In another aspect, there is provided a method for treating spinal cord injury, in a subject-in-need thereof, the method comprising administering to the subject a therapeutically effective amount of a hybrid hydrogel according to the invention or a pharmaceutical composition according to the invention. Advantageously, the administration may be carried out by locally injecting the hybrid hydrogel near the site of spinal cord injury. The injected hybrid hydrogel may be advantageously loaded with any drug useful for treating spinal cord injury, such as methylprednisolone, for example in the pores and/or core of organosilica particles (plain nanoparticles or core/shell nanoparticles) that may be mixed in the hybrid hydrogel network and/or covalently conjugated to the first hydrogel network as detailed supra, for sustained release of the drug.

In another aspect, there is provided a method for treating hernia or ulcers, in a subject-in-need thereof, the method comprising administering to the subject a therapeutically effective amount of a hybrid hydrogel according to the invention or a pharmaceutical composition according to the invention. Advantageously, the administration may be carried out by locally injecting the hybrid hydrogel at the site of hernia or ulcer, preferably at the hernia opening to close it. The injected hybrid hydrogel may be advantageously loaded with any drug useful for ancillary treating hernia or ulcers, such as anti-infection agents or anti-inflammatory drugs, for example in the pores and/or core of organosilica particles (plain nanoparticles or core/shell nanoparticles) that may be mixed in the hybrid hydrogel network and/or covalently conjugated to the first hydrogel network, as detailed supra, for sustained release of the drug.

In another aspect, there is provided a method for cardiac repair, in a subject-in-need thereof, the method comprising administering to the subject a therapeutically effective amount of a hybrid hydrogel according to the invention or a pharmaceutical composition according to the invention. The injected hybrid hydrogel may be advantageously loaded with any drug useful for cardiac repair surgeries and/or treatment, for example in the pores and/or core of organosilica particles (plain nanoparticles or core/shell nanoparticles) that may be mixed in the hybrid hydrogel network and/or covalently conjugated to the first hydrogel network, as detailed supra, for sustained release of the drug.

In another aspect, there is provided a method for treating fistulas in a subject-in-need thereof, the method comprising administering to the subject a therapeutically effective amount of a hybrid hydrogel according to the invention, or pharmaceutical composition thereof.

In another aspect, there is provided a hybrid hydrogel according to the invention, or pharmaceutical composition thereof, for use in the treatment of fistulas.

Hybrid hydrogels according to the invention therefore can find applications in in vitro and in vivo diagnostics, therapy, in cosmetics, in drug delivery, and in any other application where a release can be envisaged or prove useful.

Advantageously, unlike previous materials obtained by photo-crosslinking, or thermal gelation, the hybrid hydrogels described generally and in any variants herein may be advantageously formed in situ via Michael-type addition reaction under physiological conditions from mixing of the monomers in aqueous solution through the formation of amine bonds.

Advantageously, hybrid hydrogels described generally and in any variants herein can advantageously deliver active molecules, for example during the hydrogel degradation phase, and for example potentially assisting the healing of surrounding tissue at the site of injection.

Advantageously, hybrid hydrogels described generally and in any variants herein are preferably injectable and biodegradable.

Advantageously, hybrid hydrogels described generally and in any variants herein may undergo degradation responding to cell-secreted molecules through reductive cleavage of the linker, for example of disulfide moieties, incorporated either in the first hydrogel polymer structure; or in the organosilica matrix of particles (plain nanoparticles or core/shell nanoparticles) that may be mixed in the hybrid hydrogel network and/or covalently conjugated to the first hydrogel network; or both.

Advantageously, hybrid hydrogels described generally and in any variants herein may release molecules of interest, for example proteins, for example from the nanocapsules, through the degradation of the nanocapsule shell.

Advantageously, hybrid hydrogels described generally and in any variants herein show advantageously a rapid gelation when injected in vivo, and for example may afforded a long-lasting high mucosal elevation.

In a variant, silicone particles, preferably silicon nanoparticles, most preferably porous silicon nanoparticles, may be used in place of or in addition to the organosilica particles mentioned in any variant herein. The outer surface of silicon particles will oxidize to silicon oxide when exposed to water or an aqueous environment. As such, hybrid hydrogels of the invention may comprise silicone particles, preferably silicon nanoparticles, most preferably porous silicon nanoparticles, mixed in with the hybrid hydrogel matrix or covalently bound thereto much like the organosilica particles described herein.

Silicon porous particles are fully degradable and have the same role of the organosilicates systems (cf J. Mater. Chem. B, 2016, 4, 7050-7059; and Nature Materials 8, 331-336 (2009)). [32]

Porous silicon has exhibited considerable potential for biological applications owing to its biocompatibility, biodegradability, and the possible surface functionalization. For in vivo use, silicon nanoparticles provide attractive chemical alternatives to other quantum dots, which have been shown to be toxic in biological environments. In addition, silicon is a common trace element in humans and a biodegradation product of porous silicon, orthosilicic acid (Si(OH)₄), is the form predominantly absorbed by humans and is naturally found in numerous tissues. Furthermore, silicic acid administered to humans is efficiently excreted from the body through the urine. Porous silicon particles have been filled with therapeutics and they can be engineered to degrade in vivo into benign components that clear renally. Therefore porous silicon particles, in particular porous silicon nanoparticles, can replace or add as component of hybrid hydrogels according to the invention.

With respect with each of the uses and methods described above, any hybrid hydrogel described generally and in any variant herein may be used.

### BRIEF DESCRIPTION OF THE DRAWING

FIG. 1. Scheme of the synthesis and functionalization of BNCs containing disulfide moiety in the framework and loaded with Cyt-C inside the silica capsule (a); SEM image of the monodispersed functionalized BNCs, in the insert SEM picture of a naked nanoparticle (b); scheme of degradation after GSH exposure and release of Cyt-C (c).

FIG. 2. Synthesis of a PAAm hydrogel covalently conjugated to redox-responsive degradable organosilica nanocapsules prepared in Example 3.1 (a); scheme of the network (b); FTIR trace of dPAA (c); SEM showing the porosity of the nanocomposite (d).

FIG. 3. Mechanism of network degradation of a PAAm polymer used as first hydrogel polymer of formula (I) in the hybrid hydrogels of the present invention, upon exposure to GSH, schematic representation and pictures of the hydrogel network before and after degradation. The yellow lines represent the disulfide units; the degradation of the hydrogel

FIG. 4. Injection of a solution of PAAM and alginate (hybrid hydrogel of the present invention) stained with Methylene Blue via a surgical 23-gauge needle (a); formation of a mucosal elevation (b); gelation occurs in less than 10 minutes, achieving a solid and elastic hydrogel, adhered to the tissue.

FIG. 5. ¹H NMR spectrum of DCNS before and after light irradiation (cf. Example 1.3).

FIG. 6. Characterization of model spherical MSPs, illustrated in Example 1.3.

FIG. 7. Complete characterization of the hybrid light-sensitive spherical MSPs with light-induced cleavable linkers within the organosilica matrix, illustrated in Example 1.3.

FIG. 8. Schematic representation of the light-induced cleavability experiments and SEM images of the investigated organosilica particles comprising light-induced cleavable linkers within the organosilica matrix.

FIG. 9. FIG. 9A represents a schematic representation of the application of hybrid hydrogel according to the invention onto the duodenal mucosa, in order to interfere with nutrients adsorption and, particularly, with glucose metabolism, which is particularly active at the level of the foregut. The endoscope is advanced in the duodenum and the hybrid hydrogel is sprayed in order to cover the duodenal mucosa, while moving backwards. FIG. 9B represents endoscopic injection of a hybrid hydrogel according to the invention at the level of the lower esophageal sphincter (LES) to obtain a sphincter augmentation (increased closure strength to prevent reflux’ episodes).

FIG. 10 represents a general scheme for the treatment of fistula using a hybrid hydrogel according to the invention.

FIG. 11 represents CT scans performed in Example 6. FIG. 11A represents comparative CT scans of water, Iomeron 400, 2% sodium alginate solution, hybrid hydrogel prepared in Example 6 (with Iomeron as solvent), and hybrid hydrogel prepared according to Example 2.2 (water as solvent). FIG. 11A represents a CT scan of the fistula after injection of the hybrid hydrogel.

FIG. 12 represents comparative uniaxial compression test of 2% sodium alginate crosslinked with calcium chloride, 2% alginate/calcium/PAAm and pure PAAm hydrogel.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

The present invention and its applications can be understood further by the examples that illustrate some of the embodiments by which the inventive product and medical use may be reduced to practice. It will be appreciated, however, that these examples do not limit the invention. Variations of the invention, now known or further developed, are considered to fall within the scope of the present invention as described herein and as hereinafter claimed.

### EXAMPLES

### Example 1: Synthesis of Organosilica Particles

### 1.1. Redox-Cleavable Core/Shell Nanocapsules Synthesis

The nanocapsules used are those disclosed in E. A. Prasetyanto, A. Bertucci, D. Septiadi, R. Corradini, P. Castro-Hartmann, L. De Cola, Angew. Chem. Int. Ed. 2016, 55, 3323. [21]. This platform is composed of a silica shell able to encapsulate functional proteins in their active folding and it is engineered to degrade upon contact with a reducing agent, such as GSH present in the biological environment with a complete release of the loading.

Cytochrome C (Cyt-C) was chosen as model cargo, since its strong absorption in the visible region allowed us to investigate the release kinetics during the hydrogel degradation.

The synthesis of the BNCs, in order to prevent denaturation of the loaded active molecule, was performed following the reported reverse nano-emulsion procedure. In particular, the silica precursor, tetraethyl orthosilicate (TEOS) was added to bis[3-(triethoxysilyl)propyl]disulfide in a ratio 7:3 TEOS:bispropyldisulfide derivative, in order to introduce the redox-sensitive moiety. Well-defined and monodispersed spherical nanocapsules with a diameter of around 60±10 nm were obtained.

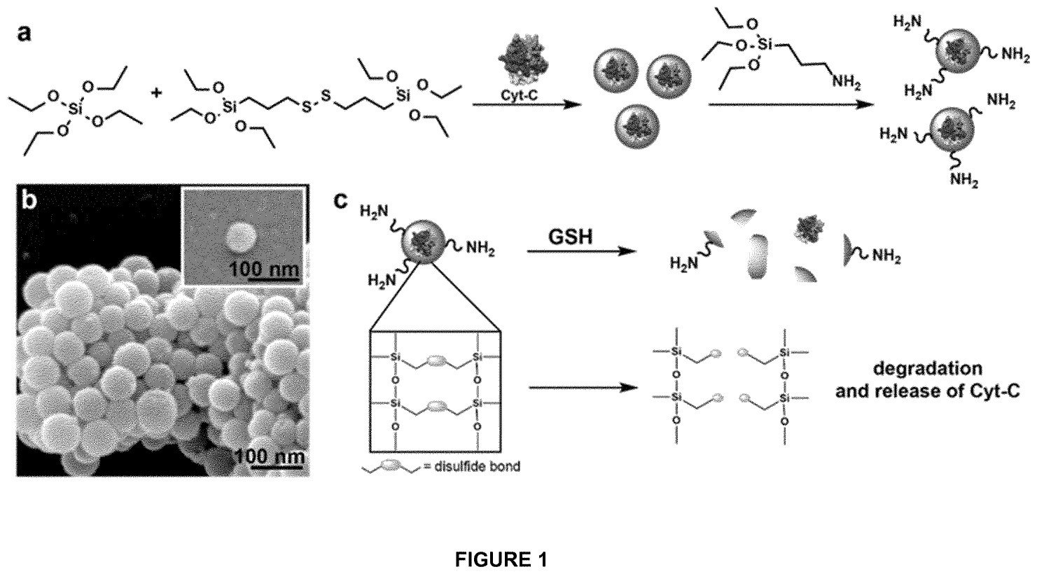

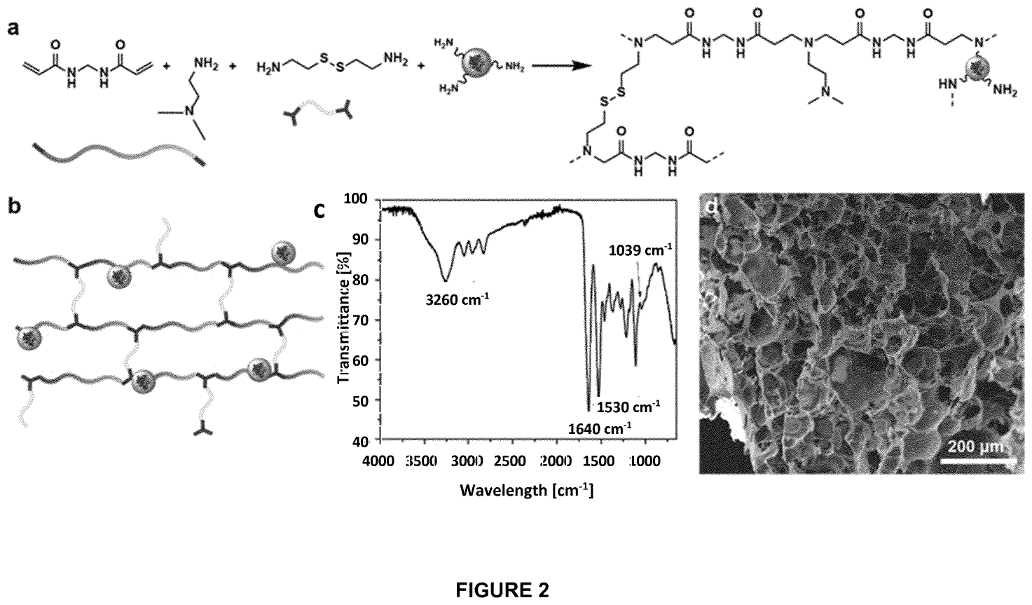

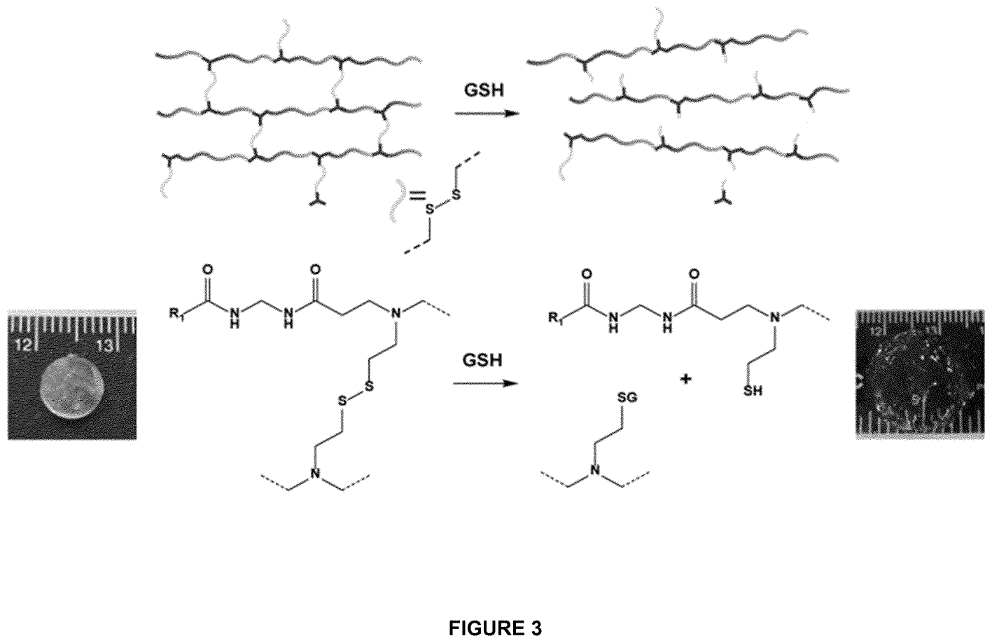

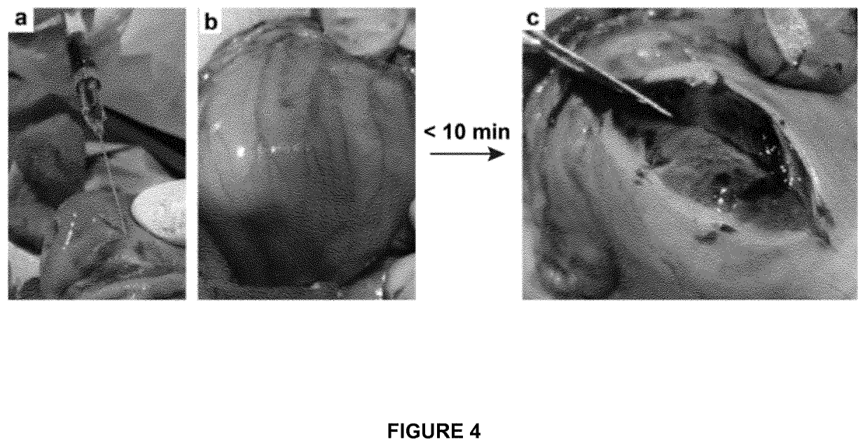

Then, the obtained pristine BNCs were functionalized with 3-aminopropyltriethoxysilane, to be able to covalently link the BNCs to the polymeric hydrogel network. A scheme of the synthesis and functionalization, as well as the SEM of the pristine and functionalized BNCs and of the degradation via GSH is displayed in FIG. 1.

The surface functionalization was confirmed by the shift from negative to positive values of the ζ-potential, from −10.5 mV of the pristine nanocapsules to +2.2 mV.

Then, the functionalized BNCs (1 mg/ml) were used to synthetize the dPAA nanocomposite hydrogel through surface-grafting of the aminated BNCs to the polyamidoamine backbone of the scaffold.

Briefly, the protocol was as follows:

Triton X-100 (7.08 mL) and n-hexanol (7.20 mL) were dissolved in Cyclohexane (30 mL). Separately, 1.20 mL of a 5 mg/mL aqueous solution of Cytochrome C from equine heart were mixed with 0.16 μL of tetraethyl orthosilicate and 0.24 mL of bis[3 (triethoxysilyl)propyl] disulfide.

After shaking, this mixture was added to the former organic medium. Eventually, 200 μL of 30% ammonia aqueous solution were added and the water-oil emulsion was stirred overnight at room temperature. After that, 80 mL of pure acetone were added to precipitate the NPs and the material was recovered by means of centrifugation, washing five times with water and one with ethanol.

The procedure can be adapted for the encapsulation of different globular proteins.

### 1.2. Redox-Cleavable Core/Shell Nanocapsules Functionalization

40 mg of breakable nanocapsules prepared in Example 1.1. are suspended in 5 mL of ethanol. 44 μL of 3-aminopropyltriethoxysilane (PM=221.37, d=0.946, 0.094 mmol) and 20 μL of triethylamine (TEA) are added to the suspension, that is stirred at R.T. for 18 hours.

The resulting core/shell nanocapsules bearing aminopropyl tether moieties at the outer surface (NPs, also designated NH2-CytC@BNPs) are then washed five times with distilled water and dried.

### 1.3. Photo-Cleavable Nanoparticles Synthesis

### 1.3.1 2-nitro-5-(((3-(triethoxysilyl)propyl)carbamoyl)oxy)benzyl (3-(triethoxysilyl)propyl)carbamate, (DCNS)

This photosensible molecule was synthesized by the reaction of the alcohol groups of the 5-hydroxy-2-nitrophenyl alcohol and Iscocyanopropyltriethoxysilane by the presence of triethylamine as catalyst (see scheme 1)

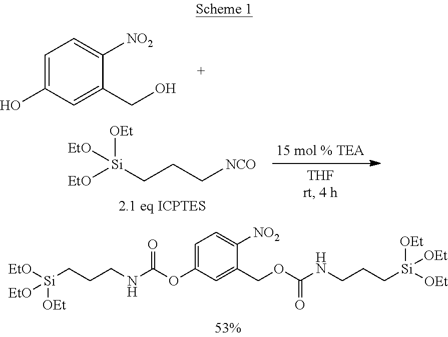

The reaction product could be obtained in a 53% of yield and had been characterized by ¹H-NMR and ¹³C-NMR, FTIR spectroscopy and ESI-mass spectrometry. Furthermore the absorption spectra had been recorded for further light breakability experiments of the linker itself.

### 1.3.2. Breakability Test on DCNS

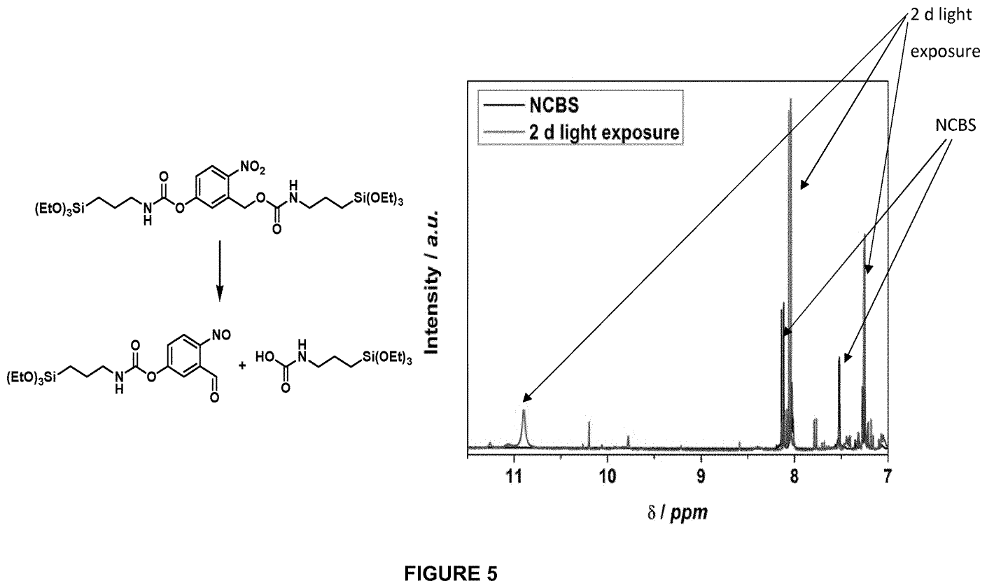

The light-induced breakability of the DCNS compound had been performed by irradiating the compound with light produced by a Hg lamp. To this purpose, the compound was dissolved in DMSO-d₆ in a NMR tube. In this way the photo degradation could be followed by recording ¹H-NMR spectra over a certain period of time. Indeed the photogradation reaction could be observed and it is indicated by the signal derived from the aldehyde proton at 10.92 ppm (FIG. 5).

### 1.3.3. Synthesis of Hybrid MSPs

Firstly, model spherical MSPs were synthesised. The model particles were synthesized according to a modified Stöber synthesis, shown in Scheme 2

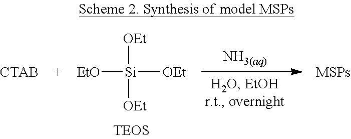 [SMILES: CCO[Si](OCC)(OCC)OCC]

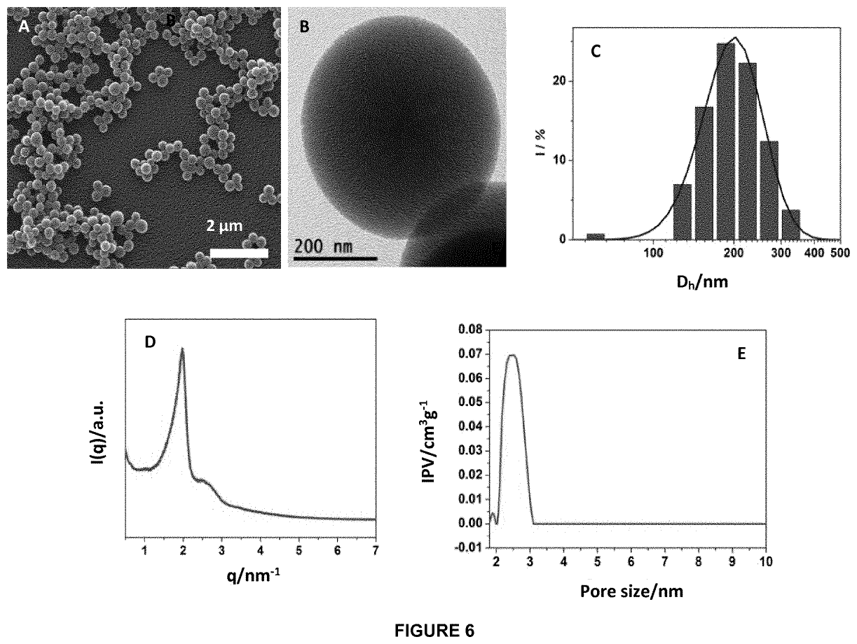

The model particles obtained were spherical characterized by an average diameter of ca 200 nm (SEM, TEM and DLS analysis in FIG. 6). Furthermore these model particles possess a hexagonal mesostructure with an estimated average pore size of 2.5 nm (see SAXS and pore size distribution in FIG. 6)

Once the standard synthesis protocol had been established, hybrid silica particles were synthesised by the co-condensation of DCNS into the silica structure. (see scheme 3)

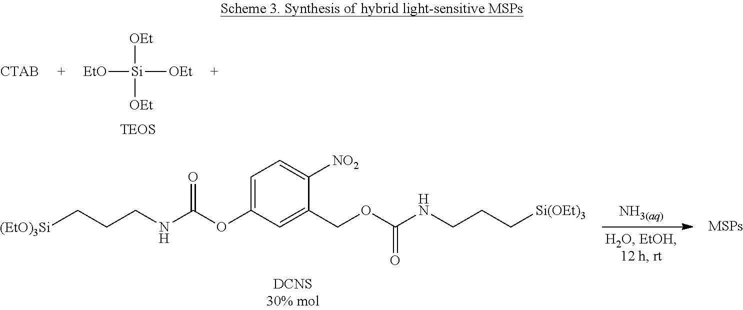

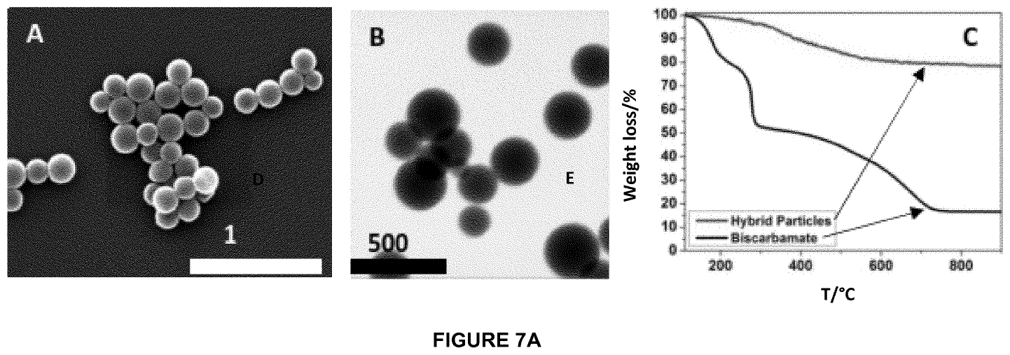

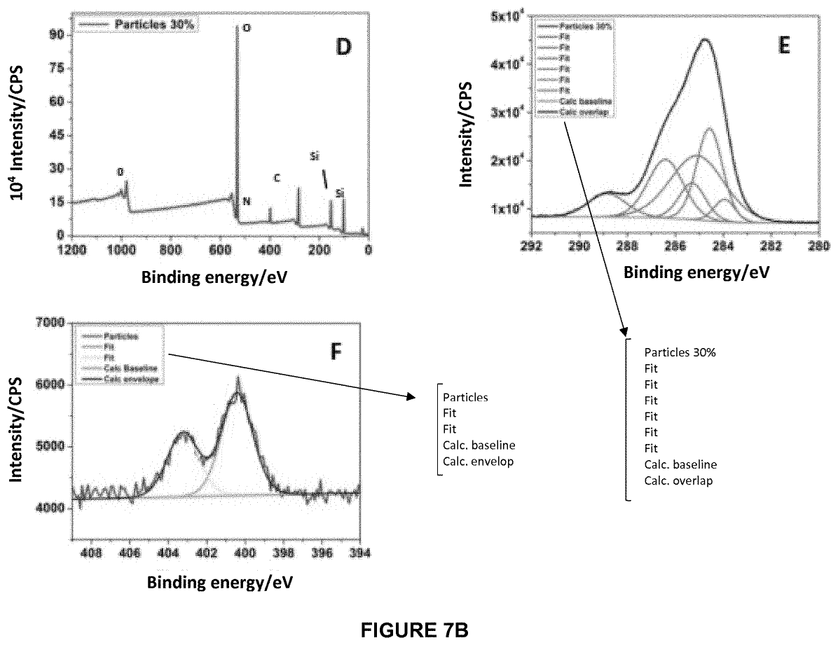

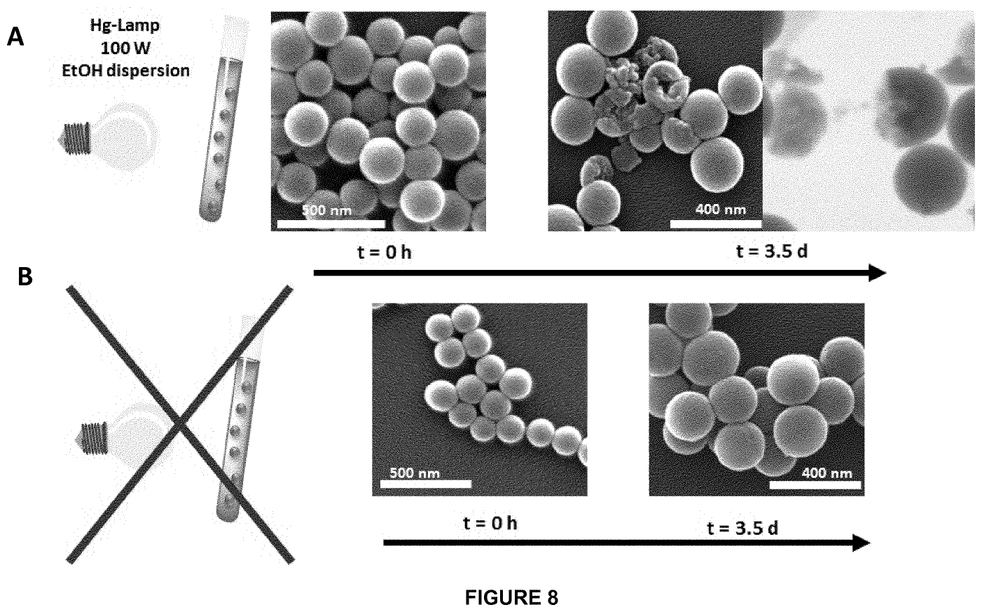

The hybrid silica particles obtained by this synthetic approach were spherical and characterized by satisfactorily monodispersity and diameter of ca. 200 nm and 20 wt. % of organic material as determined by TGA. The incorporation of the DCNS linker was proven by XPS analysis. The deconvolution of high resolution scans of the C(1s) and N(1S) indicated the presence of peaks characteristic for the functional groups present in the linker (FIG. 7).

A pH measurement of the reaction mixture before and after the addition of DCNS linker confirmed the hypothesis that the hydrolysis of the carbamate occurs in these conditions. In fact, the pH value changes from 11 to 7. The hydrolysis product could be extracted from the aqueous mixture and the recorded ¹H NMR spectrum showed the presence of a carbamic acid derivative.

The hybrid light-sensitive MSPs may be further functionalized, as described for core/shell nanocapsules above, for covalent incorporation as crosslinkers into hydrogel networks.

For example, 40 mg of hybrid light-sensitive MSPs are suspended in 5 mL of ethanol. 44 μL of 3-aminopropyltriethoxysilane (PM=221.37, d=0.946, 0.094 mmol) and 20 μL of triethylamine (TEA) are added to the suspension, that is stirred at R.T. for 18 hours. The resulting NH₂-functionalized hybrid light-sensitive MSPs (NPs, also designated NH2-MSPs herein) are then washed five times with distilled water and dried.

### 1.3.4. Light Breakability Experiments of Hybrid MSPs

In order to evaluate the light-induced breakability of hybrid light breakable silica particles a suspension of these particles in ethanol was irradiated with a Hg lamp (FIG. 7). As a control experiment, particles were kept in the dark. The SEM and STEM analyses showed that only the particles exposed to light break partially apart, whereas the non-irradiated particles showed no change in morphology. The partial breaking of particles could be explained by the fact, that during the particle formation some of the photolabile linker was hydrolized, while the remaining non-hydrolized linker was incorporated to a certain extent in the hybrid particles.

### 1.3.5. Synthesis of triethoxy(3-(4-nitro-3-((3-(triethoxysilyl)propoxy)methyl)phenoxy)propyl)silane

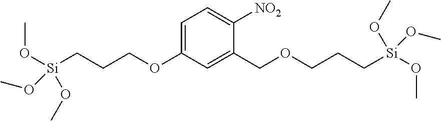

The diether compound can be prepared from 5-hydroxy-2-nitrobenzylalchol through allylation and subsequent hydrosilylation reaction, as depicted in Scheme 4. The synthetic steps are described in detail in Scheme 5.

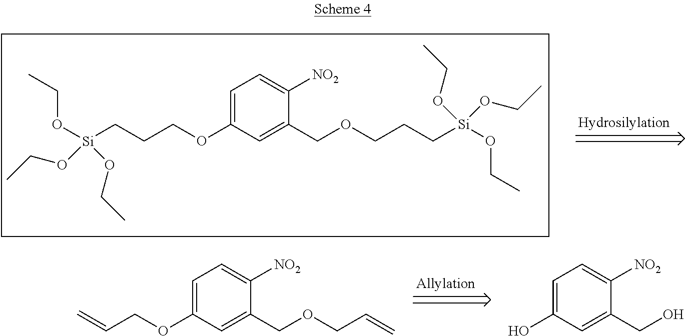

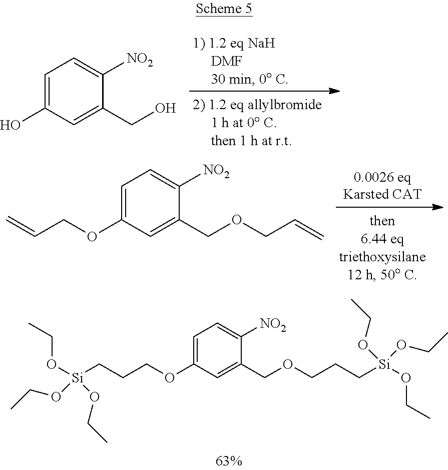

### 1.4. Photo-Cleavable Organosilica Nanoparticles Functionalization

40 mg of photo-cleavable organosilica nanoparticles prepared in Example 1.3. are suspended in 5 mL of ethanol.

44 μL of 3-aminopropyldimethoxysilane (PM=221.37, d=0.946, 0.094 mmol) and 20 μL of triethylamine (TEA) are added to the suspension, that is stirred at R.T. for 18 hours.

The resulting nanoparticles bearing aminopropyl tether moieties at the outer surface are then washed five times with distilled water and dried.

### Example 2: Synthesis of Hybrid PAAm-Alginate Hydrogels

### 2.1. pH-Degradable PAAm Hydrogels

1 g of MBA and 250 mg of GABA were weighted in a 50-ml round bottom flask. 0.85 g of diimPEHA were dissolved in 7.5 mL of distilled water and the solution was added to the flask at 45° C. under magnetic stirring until the suspension become clear. The fluid is placed in a glass vial and the temperature is then raised to 60° C.

### 2.2. pH-Degradable Hybrid Alginate-PAAm Hydrogel

The pH-degradable PAAm hydrogel prepared in Example 2.1 is mixed with a solution of sodium alginate in water. A solution of calcium chloride or any other suitable calcium salt solution in water is added and the mixture is hand-shaken until it is completely solid.

| | Sodium | PAAm/ | Calcium salt | |
|---|---|---|---|---|
| | alginate | Alginate | Conc. M | Gelation |
| PAAm hydrogel | Conc. % w/v, | weight | volume | time |
| (quantity) mL | volume added | ratio | added | (second) |
| 5 mL | 1% in H₂O, 1 | 4.5:1 | 0.5M CaCl₂ | 8 |
| | mL | | in H₂O, 0.5 | |
| | | | mL | |
| 10 mL | 1% in H₂O, 1 | 9:1 | 0.5M CaCl₂ | 15 |
| | mL | | in H₂O, 0.5 | |
| | | | mL | |
| 5 mL | 2% in H₂O, 1 | 4:1 | 0.5M CaCl₂ | 5 |
| | mL | | in H₂O, 0.5 | |
| | | | mL | |
| 10 mL | 2% in H₂O, 1 | 8.5:1 | 0.5M CaCl₂ | 8 |
| | mL | | in H₂O, 0.5 | |
| | | | mL | |

### Example 3: Synthesis of Hybrid PAAm-Alginate Hydrogels Functionalized with Organosilica Particles

### 3.1. Preparation of PAAm Hydrogel Covalently Conjugated to Redox-Responsive Degradable Organosilica Nanocapsules

200 mg of methylenbisacrylamide (MBA), 65 mg of cystamine hydrochloride and 70 μL of N,N-dimethylethylendiamine are mixed together with 1 mL of a 1 mg/mL solution of NH₂-functionalized redox-cleavable organosilica core/shell nanocapsules prepared in Example 1.2. After 48 h, the hydrogel is formed.

The procedure can be modified and other NH₂-functionalized silica nanoparticles can be used, such as responsively cleavable or non-responsively cleavable mesoporous organosilica nanoparticles. The protocol can be reproduced using the amino-functionalized photo-cleavable organosilica nanoparticles prepared in Example 1.4.

When the hydrogels were needed for in vitro experiments (i.e. GSH degradation and cellular viability and degradation), the obtained solution was transferred to glass vials (500 μl per vial) and allowed to react in static conditions at r.t. Glass vials with inner diameter of 8 mm were used as molds. The hydrogels were obtained after 48 hours.

Once obtained, the disk-shaped hydrogels were freeze-dryed and weighted. Dryed hydrogels were used to study the swelling ratio at different pH and the degradation kinetics with different concentrations of GSH. This step allowed us as well to sterilized the materials for in vitro experiments.

Sterile and ultrafiltered water was used during hydrogel preparation for in vivo tests; the synthesis was carried out in closed sterile vials and protected from bacteria contamination, the final product was assumed to be free of bacterial contamination.

### 3.2. Hybrid Alginate-PAAm Hydrogel Covalently Conjugated to Redox-Cleavable Core/Shell Organosilica Nanocapsules (Method 1)

The PAAm hydrogel covalently conjugated to organosilica nanocapsules, prepared in Example 3.1., is mixed with a solution of sodium alginate in water. A solution of calcium chloride or any other suitable calcium salt solution in water is added and the mixture is hand-shaken until it is completely solid.

| | Sodium | PAAm/ | Calcium salt | |
|---|---|---|---|---|
| NP-conjugated | alginate | Alginate | Conc. M, | Gelation |
| PAAm hydrogel | Conc. % w/v, | weight | volume | time |
| (quantity) mL | volume added | ratio | added | (min.) |
| 5 mL | 1% in H₂O, 1 | 4.5:1 | 0.5M CaCl₂ | 7 |
| | mL | | in H₂O, 0.5 | |
| | | | mL | |
| 10 mL | 1% in H₂O, 1 | 9:1 | 0.5M CaCl₂ | 13 |
| | mL | | in H₂O, 0.5 | |
| | | | mL | |
| 5 mL | 2% in H₂O, 1 | 4:1 | 0.5M CaCl₂ | 5 |
| | mL | | in H₂O, 0.5 | |
| | | | mL | |
| 10 mL | 2% in H₂O, 1 | 8.5:1 | 0.5M CaCl₂ | 8 |
| | mL | | in H₂O, 0.5 | |
| | | | mL | |

### 3.3. Hybrid Alginate-PAAm Hydrogel Covalently Conjugated to Redox-Cleavable Core/Shell Organosilica Nanocapsules (Method 2)

Hybrid PAAm-alginate hydrogels covalently conjugated to organosilica nanocapsules are prepared as previously reported (cf. Example 3.1.) using a solution of 1 mg/mL of redox-cleavable core/shell nanocapsules in sodium alginate. As such, 200 mg of methylenbisacrylamide (MBA), 65 mg of cystamine hydrochloride and 70 μL of N,N-diethylethylendiamine are mixed together with 1 mL of a 1 mg/mL solution of NH₂-functionalized redox-cleavable organosilica core/shell nanocapsules prepared in Example 1.2. in sodium alginate. When the pre-gel solution become homogeneous, a water solution of calcium chloride is added to trigger gelation.

| | Sodium | PAAm/ | Calcium salt | |
|---|---|---|---|---|
| | alginate | Alginate | Conc. M | Gelation |
| PAAm hydrogel | Conc. % w/v, | weight | volume | time |
| (quantity) mL | volume added | ratio | added | (second) |
| 5 mL | 1% in H₂O, 1 | 4.5:1 | 0.5M CaCl₂ | 8 |
| | mL | | in H₂O, 0.5 | |
| | | | mL | |
| 10 mL | 1% in H₂O, 1 | 9:1 | 0.5M CaCl₂ | 15 |
| | mL | | in H₂O, 0.5 | |
| | | | mL | |
| 5 mL | 2% in H₂O, 1 | 4:1 | 0.5M CaCl₂ | 5 |
| | mL | | in H₂O, 0.5 | |
| | | | mL | |
| 10 mL | 2% in H₂O, 1 | 8.5:1 | 0.5M CaCl₂ | 8 |
| | mL | | in H₂O, 0.5 | |
| | | | mL | |

### Example 4: Hydrogels Characterization and Uses in Non Invasive Surgery Procedures

### Degradation Kinetic of Stimuli-Responsive Hybrid Hydrogels

For redox-responsive materials, a 1 mm thick hydrogel cylinders is lyophilized and its dry weight is recorded. The hybrid hydrogel is then placed in a vial and 5 mL of a 10 μM solution of reduced GSH are added. The swelling of the hybrid hydrogel is recorded at the appropriate time-points. The experiment is repeated in triplicated and then with a solution of GSH 10 mM and with a solution of PBS as a reference.

The same procedure can be applied for pH-responsive hybrid hydrogels, using pH=4 citrate buffer for degradation and PBS as a reference.

Degradation of hybrid hydrogels covalently conjugated to organosilica particles can be examined in the presence of reduced glutathione (GSH), a disulfide reducing agents. Briefly, the lyophilized hybrid hydrogel samples are incubated at 37° C. in 2 mL of a PBS solution with a GSH concentration of 10 μM. Hybrid hydrogels without organosilica particles are incubated in PBS alone as a control.

The degradation kinetics can then be evaluated via swelling ratio (SR) measurements in time.

SR are measured by a gravimetric method. In brief, lyophilized hybrid hydrogel samples are immersed in PBS at 37° C. Then, the samples are removed from PBS at set time points (after 1 h, 6 h, 12 h, 24 h, 48 h, 72 h, 144 h), blotted free of surface water using filter paper and their swollen weights are measured on an analytical balance. The SR are then calculated as a ratio of weights of swollen hybrid hydrogel (Ws) to dried hybrid hydrogel (W), using the following equation:

$$S R = \frac{{W}_{s} - {W}_{d}}{{W}_{d}}$$

Degradation time is defined as the time where there were no longer sufficient crosslinks to maintain the 3D network and the material was completely disintegrated. Experimentally, complete degradation can be determined when a limpid solution can be observed, without solid residues.

In Vitro Cell Culturing

Cryopreserved human dermal fibroblast, adult (HDFa) are purchased from Thermo Fisher and the culture is initiated as suggested on the protocol. HDFa are grown in Medium 106 supplemented with Low Serum Growth Supplement (LSGS, Thermo Fisher). Cells are kept in 75 cm² culture flasks (Corning Inc., NY, USA) at 37° C. with a controlled atmosphere of 5% CO₂ and are grown until reaching 80 to 85% of confluence. Then, they are washed twice with PBS and treated with trypsin/EDTA solution to detach them from the flask surface. Cells are split every 2-3 days; the medium is changed every other day.

In Vitro Cell Culturing onto Hybrid Hydrogels

The hybrid hydrogel scaffolds are equilibrated by adding culture media at 37° C. HDFa are detached from the culture flask by trypsination and approximately 2.5×10⁵ cells are seeded onto the hybrid hydrogel scaffolds. Then, the samples are placed in the incubator (37° C., 5% CO₂) for about 30 minutes and fresh media is cautiously added on the top of the hybrid hydrogel to supply cells with nutrients. This is done to allow anchorage of the cells onto the scaffolds.

Cell Staining and Viability Studies

Cell viability is assessed using alamarBlue assay. Briefly, the alamarBlue solution is added to the culture medium (1:10 dilution) of unstained cells growing onto hybrid hydrogel scaffolds. After 3 h incubation, 200 μL of the media are transferred to a 96-well plate and absorbance signals generated from the dye resazurin (dark blue) being reduced to resorufin (pink) by metabolically active cells are recorded using a VICTOR X5 Multilabel Plate Reader (Perkin Elmer).

Each sample is tested in three replicates and the results are expressed as percentage of reduced alamarBlue.

The viability of cells after complete degradation of the hybrid hydrogel was measured by with a TC20 (trade mark) Automated Cell Counter (Bio-Rad).

Where required (confocal fluorescence microscopy images), HDFa are stained with Vybrant DiD (Life Technologies, Thermo Fisher Scientific, Waltham, Mass., USA), following the reported protocol, prior to seeding them onto the scaffolds.

Cell-Mediated Degradation of Hydrogel

The hybrid hydrogels are freeze-dried and weighed (W). Then 2.5×10⁵ HDFa are seeded onto the samples (see above). The cell-laden samples are collected at pre-determined time points and were freeze-dried to obtain their dry weight after degradation (W).

The cell-mediated degradation of the hybrid hydrogels, D, is calculated using the following equation:

$$D ( \% ) = \frac{{W}_{i} - {W}_{f}}{{W}_{i}} \times 100$$

A cellular hydrogels are used as degradation control.

Evaluation of the Gelation and Formation of SFC Ex Vivo

Fresh porcine stomachs are used for the ex vivo tests. The hybrid hydrogels solution is injected into the submucosal layers of the pig stomach using a 23-gauge needle. The dose can be 2 ml for each sample and the stomach is kept to a temperature of about 37° C. with a lamp to ensure simulation of in vivo conditions. Gelation of the hybrid hydrogels samples is assessed by cutting open the tissue after the desired time. The experiment may be repeated three times.

Creating Submucosal Cushion and Performing ESD in a Living Pig

The pig is fasted for 1 day before operation.

Endoscopy is performed by the surgeon.

A standard endoscope (Karl Storz, Tuttlingen, Germany) is used in the pig under general anesthesia. Both the hybrid hydrogels solution and the NS used as control contains a small amount of Methylene Blue as a color agent in order to facilitate visualization of the SFC.

After setting appropriate lesion sizes of approx. 3 cm in diameter in the porcine stomach, 810 ml of hybrid hydrogels solution and NS are injected in the stomach submucosa through the endoscope accessory channel using a 23-gauge injection needle.

The mucosal elevation due to the injected hybrid hydrogels at the target site is observed endoscopically before starting the ESD. It is compared under direct view with the elevation caused by NS during the procedure.

After injection, the ESD is performed and a circumferential mucosal incision is accomplished using a Needle knife (Olympus, Tokyo, Japan)

Injection of hybrid hydrogels and ESD may be repeated three times.

The animal is euthanized after completion of experiments; the whole procedure is followed and recorded using a Silver Scope™ Video Gastroscope (Karl Storz, Tuttlingem, Germany).

The main outcome measures are (1) the rapid gelation of hybrid hydrogels when injected into the submucosa and (2) the long-lasting SFC formed; (3) the feasibility of the dissection procedure during ESD; (3) the adhesion of hybrid hydrogels to the muscolaris layer and thus the increase of protection during the procedure and after it.

### Example 5: Treatment of Fistula and Leaks

Digestive leaks and fistulas are mostly the result of inflammatory bowel diseases or surgical manipulation of the gastrointestinal (GI) tract and their management remains challenging. Despite recent progress in interventional endoscopy that provides a minimally invasive alternative to surgery, complex acute leaks and chronic fistula remain the most difficult to treat: the healing rate is still insufficient, in particular for complex fistulas or large anastomotic leaks.

In order to validate the use of hybrid hydrogels of the invention to treat digestive leaks and chronic fistula experimental models in a large animal were created.

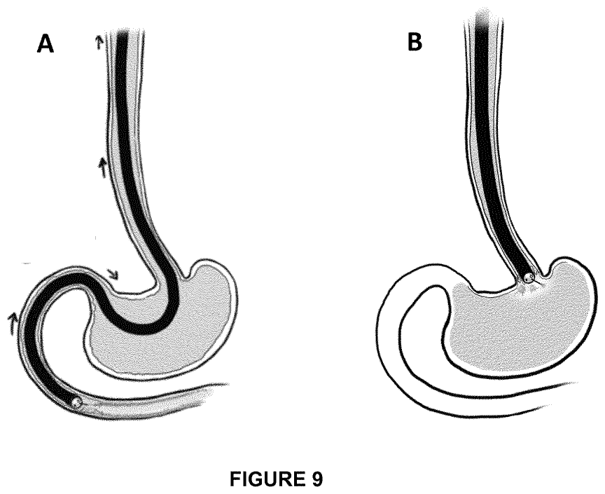

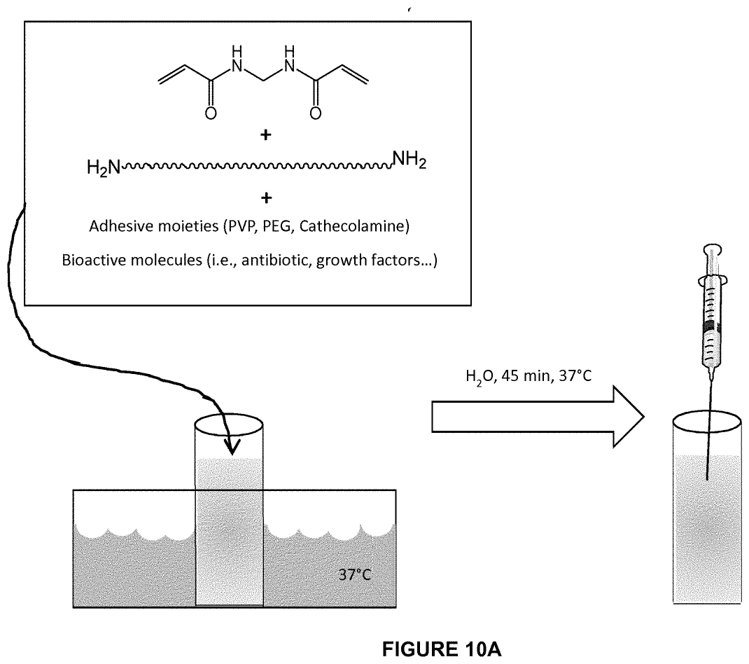

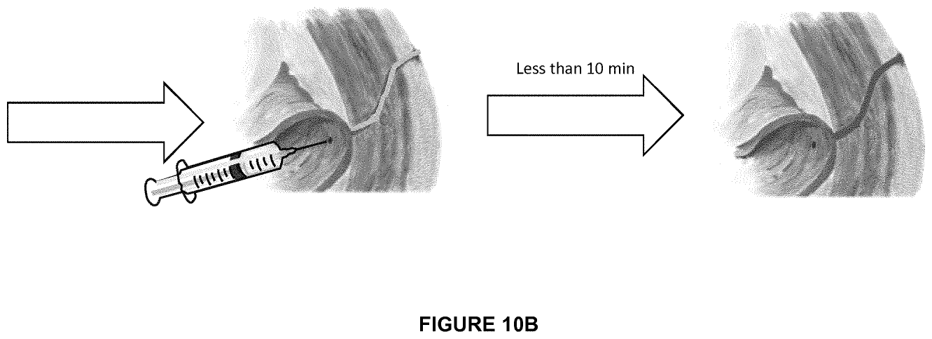

On-lay based application of the hybrid hydrogel can be used to treat gastrointestinal perforations and to create a chemical film barrier to bypass areas of the gut responsible for metabolic diseases (FIG. 9A).

Injection based hydrogel therapy can be used as filling agent to restore, heal and treat mechanical, functional and metabolic diseases: gastro-esophageal reflux disease (GERD) by restoring the lower esophageal sphincter pressure (FIG. 9B), GI fistulas by occluding the fistula tract and insulin-resistance/metabolic syndrome, by creating a physical barrier to the absorption of nutrients in crucial segments of the small bowel.

### 5.1. Treatment of Chronic Fistula

The first step of the procedure was the dissection of the lateral side of the neck of the animal to be treated (e.g. a pig).

A 5 cm skin incision was made on the neck. After dissection layer by layer the esophagus was identified and a convenient spot on the cervical esophagus 30 cm from the dental arches was chosen by transillumination using a light of the gastroscope.

A large bore needle was introduced into the esophageal lumen under endoscopic view and a guide wire fed into the needle in the esophageal lumen and retrieved by the endoscope.

A9-Fr T-tube was inserted over the guide and retrieved from the cervicotomy with the distal T part sitting into the esophagus. The catheter was then tunneled subcutaneously and secured to the skin. The same procedure was performed on the opposite side.

The T-tubes are left in place 4 weeks in order to create permanent communication between the esophageal lumen and the skin.

The following procedure was followed to treat digestive fistulas by hybrid hydrogel filling, in the above animal model of upper gastrointestinal tract fistulas. A) A drain is placed in the upper esophagus, through a cervicotomy approach, as previously described. B) The fistula path is obtained after 30 days survival. C) The fistula tract is filled with the hydrogel.

### 5.2. Treatment of Acute Fistula

In-vivo acute digestive gastro-jejunal fistula tracts were created by tubulisation of a segment of small bowel (3 cm long and 4 mm in diameter) which was then attached to the gastric wall. The small bowel cylinders were then closed at their distal end with a surgical suture.

A gastroscopy was performed by using a standard single channel endoscope to access the fistulas endoscopically.

In-vivo injection of the components of a hybrid hydrogel according to the invention was performed in 2 steps using a plug through the scope 2.8 mm plastic delivery catheter connected to a three-way valve. The hybrid hydrogel components were sodium Alginate 2%, and PAAm hydrogel (hydrogel polymer of formula (I), as described generally herein), which were injected concomitantly with Ca²⁺ to effect gelation. The PAAm hydrogel of Example 2.1 was used as hydrogel polymer of formula (I). The hybrid hydrogel gelled in vivo in a few minutes (<10 min.), thereby efficiently filling the fistula tract (and treating the fistula).

Step 1. The endoscopic delivery catheter was placed inside the proximal orifice of the fistula during the injection and removed after 2 minutes.

Step 2. A second injection was done by means of an extraction biliary catheter equipped with an inflatable balloon at its tip. The balloon was inflated in correspondence of the proximal opening of the fistula after the injection procedure and kept inflated for 2 minutes.

This allowed the components to have sufficient time to react and avoided the percolation of the solution in its liquid phase. The balloon was then deflated and the device extracted from the fistula. A careful endoscopic look was performed to confirm the presence of the gel inside the fistula.

A gastrectomy was then performed to examine the internal orifice of the fistula. The hydrogel was formed and solid and could only be removed by milking forcefully the fistula tract, which demonstrated the successful treatment of the fistula.

### 5.3. Examples

A huge challenge with existing hydrogels is the inadequate gelation time (too long, >10 minutes) which is not adapted for non-invasive surgical procedures, notably for the treatment of fistulas, because it hinders the possibility of a simple endoscopic injection of the material, considering that the hydrogel would just percolate outside the fistula.

The use of a hybrid hydrogel according to the invention, which is able to solidify in an extremely short time (less than five second), allowed to address the problem.

### 5.3.1. Comparative Example: Mixing of PAAm Hydrogel with Plasma and then Coagulation with Fibrinogen

In-vivo tests were performed to check the gelation properties of a bi-component hydrogel system made of blood-containing PAAm hydrogel of Example 2.1 and thromboplastin from rabbit (sigma). Thromboplastin was reconstituted as recommended by the producer in 10 mM CaCl₂.

A section of about 1 cm of length of the small bowel of a pig was sealed with surgical thread at the extremities. 1.3 mL of PAAm hydrogel of Example 2.1, 1 mL of porcine blood and 1 mL of reconstituted thromboplastin were mixed together and immediately injected in the bowel section. No leakage from the injection site or from the sealing was observed. After 10 minutes the section was opened to check hydrogel gelation and adhesion. The hydrogel was not formed and only small blood clots were observed.

### 5.3.2. Hybrid Hydrogel According to the Invention and Gelation with Ca²⁺

Ex-vivo tests were conducted on porcine small bowel. The bowel was explanted the day before, carefully washed, frozen for the night, de-frozen just before the tests and washed again. The bowel then divided in 1 cm long subsection with surgical thread, and reverted to have the mucosa in the external part and the mucosa inside the lumen. Hybrid hydrogels of the invention (mixture of PAAm hydrogel of Example 2.1/sodium alginate with different compositions) were injected into each section, followed by injection of the solution of Ca²⁺. Good gelation and adhesion was observed.

| | Sodium | PAAm/ | Calcium salt | |
|---|---|---|---|---|
| | alginate | Alginate | Conc. M | Gelation |
| PAAm hydrogel | Conc. % w/v, | weight | volume | time |
| (quantity) mL | volume added | ratio | added | (second) |
| 5 mL | 1% in H₂O, 1 | 4.5:1 | 0.5M CaCl₂ | 8 |
| | mL | | in H₂O, 0.5 | |
| | | | mL | |
| 10 mL | 1% in H₂O, 1 | 8.5:1 | 0.5M CaCl₂ | 15 |
| | mL | | in H₂O, 0.5 | |
| | | | mL | |
| 5 mL | 2% in H₂O, 1 | 4:1 | 0.5M CaCl₂ | 5 |
| | mL | | in H₂O, 0.5 | |
| | | | mL | |
| 10 mL | 2% in H₂O, 1 | 8:1 | 0.5M CaCl₂ | 8 |
| | mL | | in H₂O, 0.5 | |
| | | | mL | |

In contrast, No gelation was observed with injection of only PAAm hydrogel. With only alginate/Calcium gelation is observed, but no adhesion.

### 5.3.3. Injection of Pre-Gel (PAAm+Alginate) and Gelation with Ca²⁺

In-vivo tests were conducted on two fistula models obtained linking two 3 cm long sections of the intestine to the stomach of a pig. The fistulas were then accessed endoscopically.

The fistula model was prepared as described above and the distal extremity was closed. The stomach was then cut and the proximal opening of the fistula exposed. With a three-way valve, a mixture of PAAm hydrogel of Example 2.1 and 1% sodium alginate was injected inside the fistula. Then 0.1 M Ca²⁺ were injected inside the fistula. Exemplary amounts of PAAm hydrogel, sodium alginate and Ca²⁺ used in this Example are detailed in the Table in section 5.3.2 above. After one minute, hydrogel formation was checked by observing the possible percolation of fluids. The hybrid hydrogel was formed and solid. The stomach is the removed from the animal and the hydrogel is removed from the fistula applying pression to the closed extremity.

### 5.3.4. Sequential Injection of Alginate, PAAm and Gelation with Ca²⁺

### In Vitro

In the following experiment, in-vitro injection of the components of the hybrid hydrogel (Sodium Alginate 2%, PAAm hydrogel of Example 2.1) was performed through a 2.8 mm standard endoscope-compatible plastic endoscopic sheath. The procedure was to inject the solution of alginate first, then the hydrogel to clean the sheath from the alginate, and then injection of the Ca²⁺ solution was done to effect gelation of the hybrid hydrogel. The sheath was then washed with water to avoid gelation. Results were excellent, with no blocking observed and a fast in-vial gelation. Exemplary amounts of PAAm hydrogel, sodium alginate and Ca²⁺ used in this Example are detailed in the Table in section 5.3.2 above.

### In Vivo

This approach was tested in-vivo through endoscopy. A model of fistula was prepared as described above, and then Alginate, PAAm hydrogel of Example 2.1 and Ca²⁺ were sequentially injected using the procedure tested in-vial. The results were good and we observed gel formation and no blocking of the catheter.

| | Sodium | PAAm/ | Calcium salt | |
|---|---|---|---|---|
| | alginate | Alginate | Conc. M | Gelation |
| PAAm hydrogel | Conc. % w/v, | weight | volume | time |
| (quantity) mL | volume added | ratio | added | (second) |
| 5 mL | 1% in H₂O, 1 | 4.5:1 | 0.5M CaCl₂ | 8 |
| | mL | | in H₂O, 0.5 | |
| | | | mL | |
| 10 mL | 1% in H₂O, 1 | 9:1 | 0.5M CaCl₂ | 15 |
| | mL | | in H₂O, 0.5 | |
| | | | mL | |
| 5 mL | 2% in H₂O, 1 | 4:1 | 0.5M CaCl₂ | 5 |
| | mL | | in H₂O, 0.5 | |
| | | | mL | |
| 10 mL | 2% in H₂O, 1 | 8.5:1 | 0.5M CaCl₂ | 8 |
| | mL | | in H₂O, 0.5 | |
| | | | mL | |

### Example 6: Radiopacity Tests

A hybrid hydrogel according to the invention containing a contrast solution was also used to check the visualization via CT scan.

Having a material that is radiopaque is of great interest: this allows the surgeons to check if the fistula is completely filled with the material and to follow in time the degradation of the hydrogel.

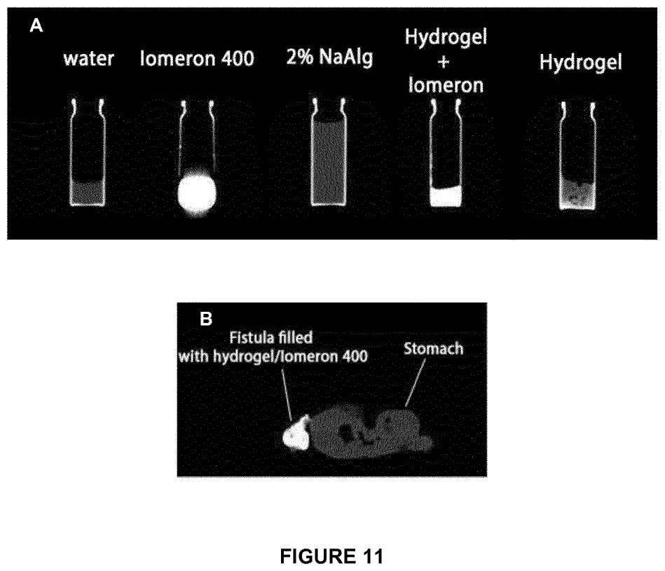

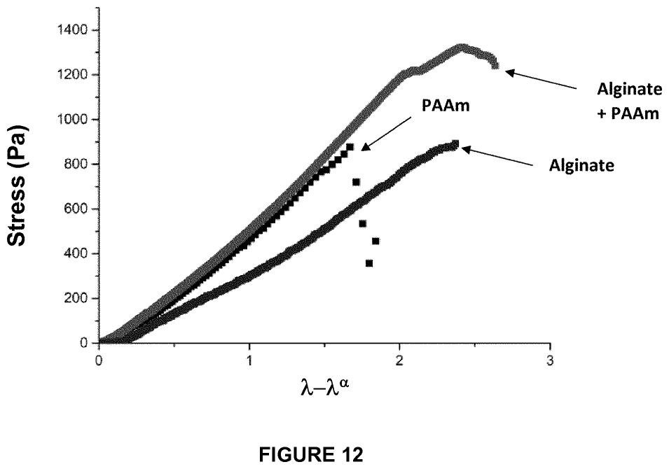

A conventional contrast agent, Iomeron (iodium-based contrast agent), was used as a solvent for the synthesis of the hybrid hydrogel, which was prepared according to Example 2.2, replacing water with Iomeron. The resulting pre-gel was injected inside a fistula (model fistula described above), followed by CaCl₂ for gelation. The hybrid hydrogel formed showed good contrast compatible with the real application, as evidenced in FIG. 11B.

| | Sodium | PAAm/ | Calcium salt | |
|---|---|---|---|---|
| | alginate | Alginate | Conc. M | Gelation |
| PAAm hydrogel | Conc. % w/v, | weight | volume | time |
| (quantity) mL | volume added | ratio | added | (second) |
| 5 mL | 1% in H₂O, 1 | 4.5:1 | 0.5M CaCl₂ | 7 |
| | mL | | in H₂O, 0.5 | |
| | | | mL | |
| 10 mL | 1% in H₂O, 1 | 9:1 | 0.5M CaCl₂ | 15 |
| | mL | | in H₂O, 0.5 | |
| | | | mL | |
| 5 mL | 2% in H₂O, 1 | 4:1 | 0.5M CaCl₂ | 5 |
| | mL | | in H₂O, 0.5 | |
| | | | mL | |
| 10 mL | 2% in H₂O, 1 | 8.5:1 | 0.5M CaCl₂ | 8 |
| | mL | | in H2O, 0.5 | |
| | | | mL | |

### LIST OF REFERENCES

[1] J. Ferlay, H. R. Shin, F. Bray, D. Forman, C. Mathers, D. M. Parkin, Int. J. Cancer 2010, 127, 2893.
[2] a) H. H. Hartgrink, E. P. M. Jansen, N. C. T. van Grieken, C. J. H. van de Velde, The Lancet, 374, 477; b) J. A. Ajani, D. J. Bentrem, S. Besh, T. A. D'Amico, P. Das, C. Denlinger, M. G. Fakih, C. S. Fuchs, H. Gerdes, R. E. Glasgow, J. A. Hayman, W. L. Hofstetter, D. H. Ilson, R. N. Keswani, L. R. Kleinberg, W. M. Korn, A. C. Lockhart, K. Meredith, M. F. Mulcahy, M. B. Orringer, J. A. Posey, A. R. Sasson, W. J. Scott, V. E. Strong, T. K. Varghese, G. Warren, M. K. Washington, C. Willett, C. D. Wright, N. R. McMillian, H. Sundar, J. Natl. Compr. Canc. Netw. 2013, 11, 531; c) A. G. Zauber, S. J. Winawer, M. J. O'Brien, I. Lansdorp-Vogelaar, M. van Ballegooijen, B. F. Hankey, W. Shi, J. H. Bond, M. Schapiro, J. F. Panish, E. T. Stewart, J. D. Waye New Engl. J. Med. 2012, 366, 687.
[3] S. Coda, S.-Y. Lee, T. Gotoda, Gut and Liver 2007, 1, 12.
[4] a) S. Oka, S. Tanaka, I. Kaneko, R. Mouri, M. Hirata, T. Kawamura, M. Yoshihara, K. Chayama, Gastrointest. Endosc. 2006, 64, 877; b) T. Gotoda, H. Yamamoto, M. R. Soetikno, J. Gastroenterol. 2006, 41, 929.
[5] a) M. Fujishiro, N. Yahagi, K. Kashimura, T. Matsuura, M. Nakamura, N. Kakushima, S. Kodashima, S. Ono, K. Kobayashi, T. Hashimoto, N. Yamamichi, A. Tateishi, Y. Shimizu, M. Oka, M. Ichinose, M. Omata, Gastrointest. Endosc. 2005, 62, 933; b) K. B. Cho, W. J. Jeon, J. J. Kim, World Journal of Gastroenterology: WJG 2011, 17, 2611.
[6] a) L. Yu, W. Xu, W. Shen, L. Cao, Y. Liu, Z. Li, J. Ding, Acta Biomater. 2014, 10, 1251; b) M. Fujishiro, N. Yahagi, K. Kashimura, Y. Mizushima, M. Oka, S. Enomoto, N. Kakushima, K. Kobayashi, T. Hashimoto, M. Iguchi, Y. Shimizu, M. Ichinose, M. Omata, Endoscopy 2004, 36, 579.
[7] K. J. Kang, B.-H. Min, J. H. Lee, E. R. Kim, C. O. Sung, J. Y. Cho, S. W. Seo, J. J. Kim, Dig. Dis. Sci. 2013, 58, 1491.
[8] R. T. Tran, M. Palmer, S.-J. Tang, T. L. Abell, J. Yang, Gastrointest. Endosc. 2012, 75, 1092.
[9] A. O. Ferreira, J. Moleiro, J. Torres, M. Dinis-Ribeiro, Endoscopy International Open 2016, 4, E1.
[10] a) T. Uraoka, T. Fujii, Y. Saito, T. Sumiyoshi, F. Emura, P. Bhandari, T. Matsuda, K.-I. Fu, D. Saito, Gastrointest. Endosc. 2005, 61, 736; b) M. Fujishiro, N. Kakushima, S. Kodashima, K. Kashimura, T. Matsuura, Y. Muraki, A. Tateishi, M. Omata, Digestive Endoscopy 2007, 19, 26.
[11]A. B. Feitoza, C. J. Gostout, L. J. Burgart, A. Burkert, L. J. Herman, E. Rajan, Gastrointest. Endosc., 57, 41.
[12]N. Yoshida, Y. Naito, M. Kugai, K. Inoue, K. Uchiyama, T. Takagi, T. Ishikawa, O. Handa, H. Konishi, N. Wakabayashi, N. Yagi, S. Kokura, Y. Morimoto, K. Kanemasa, A. Yanagisawa, T. Yoshikawa, J. Gastroenterol. Hepatol. 2011, 26, 286.
[13] Y. Matsui, M. Inomata, K. Izumi, K. Sonoda, N. Shiraishi, S. Kitano, Gastrointest. Endosc. 2004, 60, 539.
[14] T. Hayashi, T. Matsuyama, K. Hanada, K. Nakanishi, M. Uenoyama, M. Fujita, M. Ishihara, M. Kikuchi, T. Ikeda, H. Tajiri, Journal of Biomedical Materials Research Part B: Applied Biomaterials 2004, 71B, 367.
[15] I. Kumano, M. Ishihara, S. Nakamura, S. Kishimoto, M. Fujita, H. Hattori, T. Horio, Y. Tanaka, K. Hase, T. Maehara, Gastrointest. Endosc. 2012, 75, 841.
[16] J. L. Ifkovits, J. A. Burdick, Tissue Eng. 2007, 13, 2369.
[17] F. Fiorini, E. A. Prasetyanto, F. Taraballi, L. Pandolfi, F. Monroy, I. López-Montero, E. Tasciotti, L. De Cola, Small, accepted.
[18] L. Maggini, I. Cabrera, A. Ruiz-Carretero, E. A. Prasetyanto, E. Robinet, L. De Cola, Nanoscale 2016, 8, 7240.
[19] S. Mura, J. Nicolas, P. Couvreur, Nat Mater 2013, 12, 991.
[20] a) F. Yang, J. Wang, G. Peng, S. Fu, S. Zhang, C. Liu, J. Mater. Sci. Mater. Med. 2012, 23, 697; b) M. Kar, Y.-R. Vernon Shih, D. O. Velez, P. Cabrales, S. Varghese, Biomaterials 2016, 77, 186.
[21] E. A. Prasetyanto, A. Bertucci, D. Septiadi, R. Corradini, P. Castro-Hartmann, L. De Cola, Angew. Chem. Int. Ed. 2016, 55, 3323.
[22] a) G. D. Nicodemus, S. J. Bryant, Tissue Engineering. Part B, Reviews 2008, 14, 149; b) H. Henning Winter, in Encyclopedia of Polymer Science and Technology, John Wiley & Sons, Inc., 2002.
[23] A. J. Parker, N. Kharasch, Chem. Rev. 1959, 59, 583.
[24] M. E. Smithmyer, L. A. Sawicki, A. M. Kloxin, Biomaterials Science 2014, 2, 634.
[25] P. Ferruti, S. Bianchi, E. Ranucci, F. Chiellini, V. Caruso, Macromol. Biosci. 2005, 613.
[26] M. P. Lutolf, J. A. Hubbell, Biomacromolecules 2003, 4, 713.
[27] C. D. Pritchard, T. M. O'Shea, D. J. Siegwart, E. Calo, D. G. Anderson, F. M. Reynolds, J. A. Thomas, J. R. Slotkin, E. J. Woodard, R. Langer, Biomaterials 2011, 32, 587.
[28] M. K. Nguyen, D. K. Park, D. S. Lee, Biomacromolecules 2009, 10, 728.
[29] S. J. Rowan, S. J. Cantrill, G. R. L. Cousins, J. K. M. Sanders, J. F. Stoddart, Angew. Chem. Int. Ed., 2002, 41, 898.
[30] Voigt J et al. “Hyaluronic acid derivatives and their healing effect on burns, epithelial surgical wounds, and chronic wounds: a systematic review and meta-analysis of randomized controlled trials.” Wound Repair Regen. 2012 May-June; 20(3):317-31.
[31] Kuen Yong Lee et al. “Alginate: properties and biomedical applications” Prog Polym Sci. 2012 January; 37(1): 106-126.
[32] (a) J. Mater. Chem. B, 2016, 4, 7050-7059; and (b) Nature Materials 8, 331-336 (2009).

1. A hybrid hydrogel comprising:
A) A first hydrogel polymer comprising monomers of formula (I): 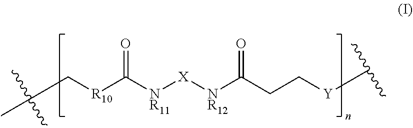
wherein
n is an integer representing the number of monomers (I) in the hydrogel polymer;
for each occurrence of the bracketed structure n, Y independently represents:
a molecular crosslinker for connecting at least a monomer of formula (I) in the framework to at least another monomer of formula (I) in another framework through a linker having the following structure:
*-R¹-L₁-R²-*;
wherein:
each occurrence of *-R¹-L₁-R²-* independently represents a responsively cleavable moiety or a non-cleavable moiety;
each occurrence of * denotes a point of attachment of the linker to a monomer of formula (I) in the hydrogel's framework;
L₁ represents a responsively cleavable covalent bond, a moiety containing a responsively cleavable covalent bond and/or a stable covalent bond;
R¹ and R² independently represent an optionally substituted C1-20 alkylenyl moiety, an optionally substituted C1-20heteroalkylenyl moiety, an optionally substituted ethenylenyl moiety, —C≡C— or an optionally substituted phenyl moiety, wherein the C1-20 alkylenyl, C1-20 heteroalkylenyl or ethenylenyl moiety may bear one or more substituents selected from halogen or —OR where R may represent H or C1-6 alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;
wherein *-R¹-L₁-R²-* may independently comprise sugar derivatives such as mannose, hyaluronic acid derivatives, collagene, aminoacids or peptides;
or
a group of formula
*-R₇(R₈)-*
wherein
the * symbols denote the points of attachment of Y within the monomer backbone of formula (I);
R⁷ represents N,
R⁸ represents an optionally substituted C1-20 alkyl, C1-20alkenyl or C1-20alkynyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety, wherein each of the foregoing C1-20 alkyl, C1-20alkenyl, C1-20alkynyl, C1-20heteroalkyl or C1-20alkylphenyl moieties may bear one or more substituents selected from halogen, —OR, —CO₂R or —N(Rp)2; where R may represent H or C1-6alkyl and each occurrence of Rp may independently represent H or C1-6alkyl; and the phenyl moiety may bear one or more substituents independently selected from halogen, C1-6alkyl, —NO₂, —CN, isocyano, —ORp, —N(Rp)2 wherein each occurrence of Rp independently represents H, C1-6alkyl or C1-6 alkoxy; wherein R⁸ may be optionally crosslinked to another monomer of formula (I) in another hydrogel polymer chain; or
a hyaluronic acid, alginic acid, peptide, cellulose, amino acid, sugar such as glucose, lactose or mannose derivatives, or oligonucleotide moiety;
for each occurrence of the bracketed structure n, R₁₀ independently represents an optionally substituted C1-20 alkylenyl moiety, wherein the C1-20 alkylenyl moiety may bear one or more substituents selected from halogen or —OR where R may represent H or C1-6alkyl;
for each occurrence of the bracketed structure n, R₁₁ and R₁₂ independently represent H, an optionally substituted C1-20 alkyl, C1-20alkenyl or C1-20alkynyl moiety, an optionally substituted C1-20heteroalkyl moiety, or an optionally substituted phenyl moiety, wherein each of the foregoing C1-20 alkyl, C1-20alkenyl, C1-20alkynyl or C1-20heteroalkyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;
for each occurrence of the bracketed structure n, X independently represents an optionally substituted C1-20 alkylenyl moiety, wherein the C1-20 alkylenyl moiety may bear one or more substituents selected from halogen or —OR where R may represent H or C1-6alkyl; and
B) at least a polysaccharide-based hydrogel, preferably an alginate-based hydrogel, preferably a hydrogel based on an alginate polymer having formula (II): 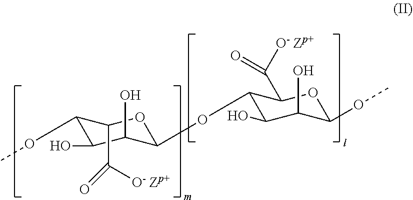
wherein each occurrence of Z independently represents a counterion such as Ca, Mg, Na, K, Li, Rb and m, 1, p are independently integers.

2. Hybrid hydrogel according to claim 1, wherein at least in a subset of bracketed structures n:
L₁ represents independently a responsively cleavable covalent bond selected from: 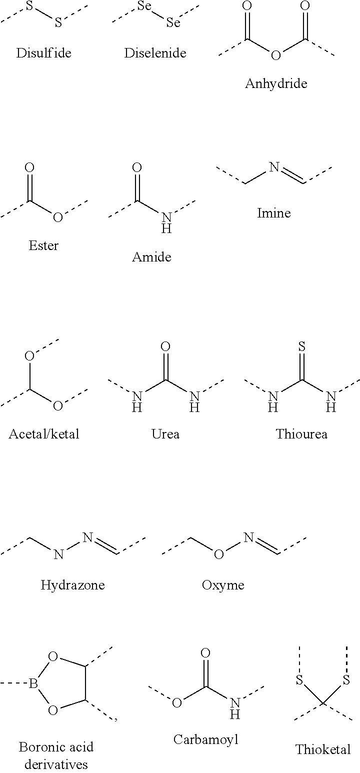
a light-induced breakable group or a photo-responsive group; or
*-R¹-L₂-R²-* represents:
i) a pH-cleavable linker comprising to imine groups conjugated with an aromatic group such as phenyl, preferably a linker comprising a para di-imino phenyl group;
ii) a pH-cleavable linker of formula: 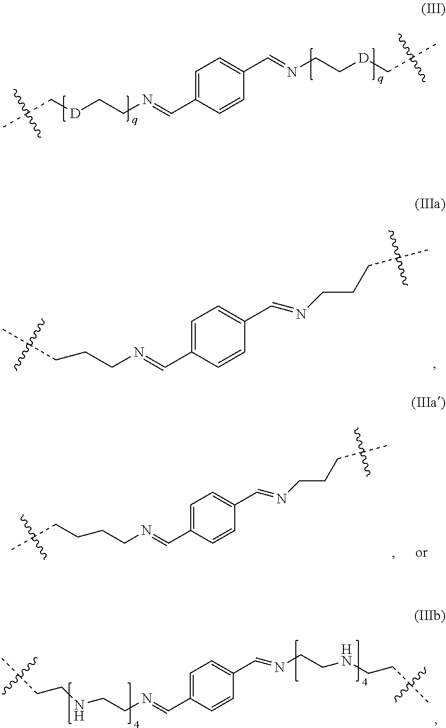
wherein each occurrence of q independently represents an integer, for example 1-6; and
D represents independently for each occurrence a C1-C3 alkylenyl moiety, or —N(Rz)— wherein Rz represents H or C1-6alkyl;
iii) a light-induced cleavable linker having formula: 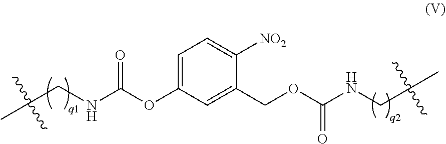
wherein q1 and q2 independently represent an integer from 1 to 6, preferably from 1 to 3. For example, q1 and q2 may both represent an integer from 1 to 6, preferably from 1 to 3, more preferably q1=q2=3; or
iv) a responsively cleavable moiety selected from: 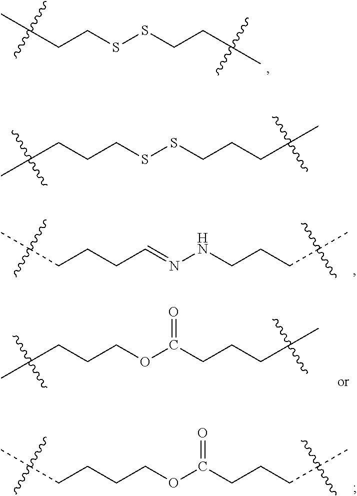
v) a moiety comprising a sugar derivative such as mannose, a hyaluronic acid derivative, collagene, an aminoacid or a peptide moiety.

3. Hybrid hydrogel according to claim 1, wherein in the linker having the structure *-R¹-L₁-R²-*, R¹ and R² are identical, and each represent —CH₂—, —(CH₂)₂—, —(CH₂)₃—, —(CH₂)₄—, or phenyl.

4. Hybrid hydrogel according to any one of claims 1 to 3 wherein in the group of formula
*-R₇(R₈)-*,
R₇ is N and
R⁸ represents a C1-C6 alkyl substituted with a carboxyl moiety, a C1-C6 alkyl substituted with one or more hydroxyl groups, C1-C6 alkoxy, C1-C6 alkyl substituted with —N(Rp)2 wherein each occurrence of Rp independently represents a C1-6alkyl.

5. Hybrid hydrogel according to any one of claims 1 to 4 wherein in the group of formula *-R₇(R₈)-*, R⁷ may be may be N and and R⁸ may be independently selected from the group comprising:

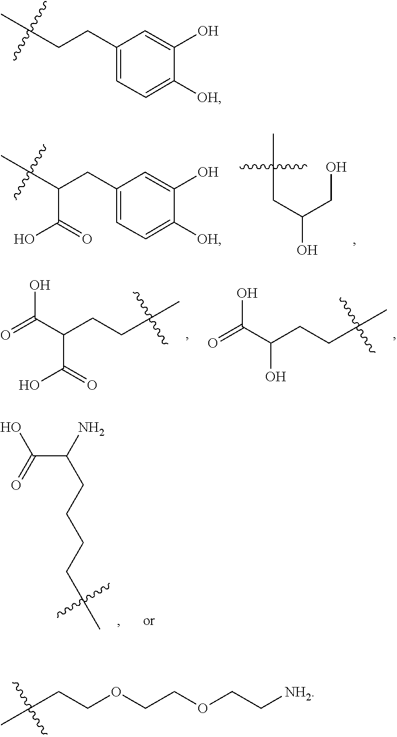

6. Hybrid hydrogel according to any one of claims 1 to 5 wherein at least a subset of occurrences of Y in the first hydrogel polymer represents *-N(R⁸)-* wherein R⁸ represents a C1-20alkyl or C1-20heteroalkyl moiety, preferably C1-6alkyl or C1-6heteroalkyl, most preferably C1-6alkyl, bearing:
(i) an organosilica nanoparticle; or
(ii) an organosilica nanocapsule having a core/shell structure, and a molecule of interest or bioactive macromolecule or bioactive macromolecule cluster encapsulated within said nanocapsule, wherein the bioactive macromolecule(s) or macromolecule cluster(s) within the nanocapsule is/are preferably in an active conformation;
wherein the organosilica matrix of the nanoparticle or nanocapsule may be disintegrable and may contain responsively cleavable bridges #-R³-L₂-R⁴-# between Si atoms within the organosilica framework;
preferably the organosilica matrix of the disintegrable organosilica nanoparticle or core/shell nanocapsule may be porous, most preferably mesoporous;
wherein:
each occurrence of # denotes a point of attachment to a Si atom in the organosilica material's framework;
L₂ represents a responsively cleavable covalent bond; and
R³ and R⁴ independently represent an optionally substituted C1-20 alkylenyl moiety, an optionally substituted C1-20 heteroalkylenyl moiety, an optionally substituted ethenylenyl moiety, —C≡C— or an optionally substituted phenyl moiety, wherein the C1-20alkylenyl, C1-20 heteroalkylenyl or ethenylenyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —OR^{p}, —N(R^{p})₂ wherein each occurrence of R^{p} independently represents H or C1-6alkyl; and
wherein the nanoparticle or nanocapsule outer surface comprises one or more groups of formula
#-R⁵R⁶
wherein
each occurrence of # denotes a point of attachment to a Si atom at the outer surface of the hybrid organosilica material's framework;
each occurrence of R⁵ independently represents an optionally substituted C1-20alkylenyl moiety, an optionally substituted C1-20heteroalkylenyl moiety, an optionally substituted ethenylenyl moiety, —C≡C— or an optionally substituted phenyl moiety, wherein the C1-20alkylenyl, C1-20heteroalkylenyl or ethenylenyl moiety may bear one or more substituents selected from halogen or —OR where R may represent H or C1-6alkyl, and the phenyl moiety may bear one or more substituents independently selected from halogen, C1-6alkyl, —NO2, —CN, isocyano, —ORp, —N(Rp)2 wherein each occurrence of Rp independently represents H or C1-6alkyl; and
each occurrence of R⁶ independently represents —OR, —SR or —N(Rf)₂;
preferably —N(Rf)₂; wherein each occurrence of R and Rf independently represents H or C1-6alkyl.

7. Hybrid hydrogel according to any one of claims 1 to 6 wherein
R₁₀ represents CH— or CH—CH₂; and
R₁₁ and R₁₂ independently represent H or C1-C6 alkyl.

8. Hybrid hydrogel according to 6 wherein at least a subset of nanocapsules bound to the first hydrogel polymer are further crosslinked via one or more #-R⁵R⁶ groups to another first hydrogel polymer of formula I.

9. Hybrid hydrogel according to claim 6 or 8 wherein the nanoencapsulated molecule is selected from proteins, enzymes, antibodies, peptides, DNA, RNA, PNA, gene fragments and small molecules with or without pharmaceutical activity; preferably proteins, enzymes, antibodies, peptides, DNA, RNA, PNA and gene fragments.

10. Hybrid hydrogel according to any one of claims 6 to 9, wherein L₂ represents independently a responsively cleavable covalent bond selected from: 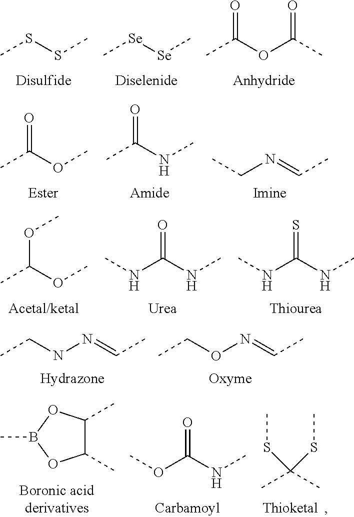
a light breakable group or a photo-responsive group, or
#-R³-L₂-R⁴-# represents:
i) a pH-cleavable linker comprising to imine groups conjugated with an aromatic group such as phenyl, preferably a linker comprising a para di-imino phenyl group;
ii) a pH-cleavable linker of formula: 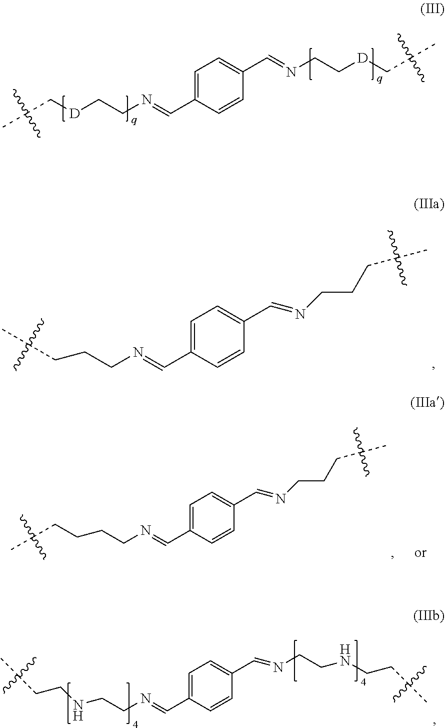
wherein each occurrence of q independently represents an integer, for example 1-6; and
D represents independently for each occurrence a C1-C3 alkylenyl moiety, or —N(Rz)— wherein Rz represents H or C1-6alkyl;
iii) a light-induced cleavable linker having formula: 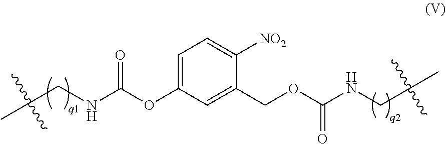
wherein q1 and q2 independently represent an integer from 1 to 6, preferably from 1 to 3. For example, q1 and q2 may both represent an integer from 1 to 6, preferably from 1 to 3, more preferably q1=q2=3; or
iv) a responsively cleavable moiety selected from:

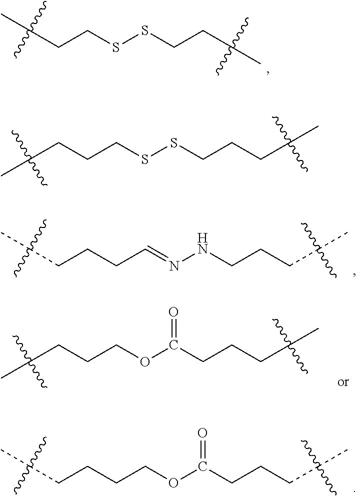

11. Hybrid hydrogel of any one of claims 1 to 10, wherein the organosilica particles bound to the hydrogel polymer has a diameter between 25 nanometers and 500 nanometers.

12. Hybrid hydrogel of any one of claims 1 to 11, wherein the hydrogel is non covalently mixed with
(i) an organosilica nanoparticle; and/or
(ii) an organosilica nanocapsule having a core/shell structure, and a molecule of interest or bioactive macromolecule or bioactive macromolecule cluster encapsulated within said nanocapsule, wherein the bioactive macromolecule(s) or macromolecule cluster(s) within the nanocapsule is/are preferably in an active conformation;
wherein the organosilica nanoparticle or nanocapsule is as defined in claim 6.

13. A pharmaceutical or cosmetic composition comprising a hydrogel of any one of claims 1 to 12, and a pharmaceutically or cosmetically acceptable carrier.

14. A method for preparing a hybrid hydrogel of any one of claims 1 to 12, comprising steps of:
a) dissolving in water or alcoholic solutions:
a monomer precursor of formula (IV) 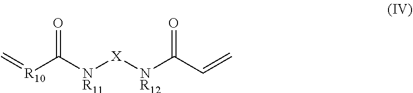
at least one molecular crosslinker precursor having the structure A-R¹-L₁-R²-A,
optionally organosilica nanoparticles optionally bearing amino-containing tether groups at the outer surface; or organosilica core/shell nanocapsules optionally bearing amino-containing tether groups at the outer surface and encapsulating a bioactive macromolecule or bioactive macromolecule cluster, and/or another molecule of interest that may or may not have biological activity and/or pharmaceutical or cosmetic activity; wherein the bioactive macromolecule or bioactive macromolecule cluster encapsulated within the nanocapsule is preferably in active conformation; and
Optionally, a selected precursor of formula B—R⁸;
b) adding a solution of alginate, for example an aqueous solution of sodium alginate, which may be added concomitantly with step a) or separately from step a);
c) Stirring the solution obtained in step b), at any appropriate temperature, thereby allowing the polymerization carried out to form the hydrogel,
d) Optionally adding a suitable organic solvent, thereby precipitating the hydrogel:
wherein:
each occurrence of A independently represents a nucleophilic moiety, preferably —N(Rf)₂ wherein each occurrence of Rf may represent H or C1-6alkyl;
B independently represents a nucleophilic moiety, preferably —N(Rf)₂ wherein each occurrence of Rf may represent H or C1-6alkyl;
L₁ independently represents a responsively cleavable covalent bond, a moiety containing a responsively cleavable covalent bond and/or a stable covalent bond; and
R¹ and R² independently represent an optionally substituted C1-20 alkylenyl moiety, an optionally substituted C1-20heteroalkylenyl moiety, an optionally substituted ethenylenyl moiety, —C≡C— or an optionally substituted phenyl moiety, wherein the C1-20 alkylenyl, C1-20 heteroalkylenyl or ethenylenyl moiety may bear one or more substituents selected from halogen or —OR where R may represent H or C1-6 alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl; wherein *-R¹-L₁-R²-* may independently comprise a sugar derivative such as mannose, a hyaluronic acid derivative, collagene, an aminoacid or a peptide moiety;
R₁₀ independently represents an optionally substituted C1-20 alkylenyl moiety, wherein the C1-20 alkylenyl moiety may bear one or more substituents selected from halogen or —OR where R may represent H or C1-6alkyl;
R₁₁ and R₁₂ independently represent an optionally substituted C1-20 alkyl, C1-20alkenyl or C1-20alkynyl moiety, an optionally substituted C1-20heteroalkyl moiety, or an optionally substituted phenyl moiety, wherein each of the foregoing C1-20 alkyl, C1-20alkenyl, C1-20alkynyl or C1-20heteroalkyl moiety may bear one or more substituents selected from halogen or —OR where R may represent H or C1-6alkyl, and the phenyl moiety may bear one or more substituents independently selected from halogen, C1-6alkyl, —NO₂, —CN, isocyano, —ORp, —N(Rp)2 wherein each occurrence of Rp independently represents H or C1-6alkyl;
X independently represents an optionally substituted C1-20 alkylenyl moiety, wherein the C1-20 alkylenyl moiety may bear one or more substituents selected from halogen or —OR where R may represent H or C1-6alkyl; and
R⁸ may independently represent:
an optionally substituted C1-20 alkyl moiety, a C1-20 alkyl optionally substituted with carboxyl moiety, an optionally substituted C1-20heteroalkyl moiety, an optionally substituted C1-20alkylphenyl moiety or an optionally substituted phenyl moiety, wherein each of the foregoing C1-20 alkyl, C1-20heteroalkyl or C1-20alkylphenyl moieties may bear one or more substituents selected from halogen, —OR, —CO₂R or —N(Rp)2 where R may represent H or C1-6alkyl, and each occurrence of Rp may independently represent H or C1-6alkyl; and the phenyl moiety may bear one or more substituents independently selected from halogen, C1-6alkyl, —NO2, —CN, isocyano, —ORp, —N(Rp)2 wherein each occurrence of Rp independently represents H, C1-6alkyl or C1-6 alkoxy;
the residue of the corresponding amino acid H₂NR⁸;
a C1-C6 alkyl substituted with a carboxyl moiety, a C1-C6 alkyl substituted with one or more hydroxyl groups, C1-C6 alkoxy, C1-C6 alkyl substituted with —N(Rp)2 wherein each occurrence of Rp independently represents a C1-6alkyl;
a C1-C6 alkyl substituted with —N(Rp)2 wherein each occurrence of Rp independently represents a C1-6alkyl;
a C2 alkyl substituted with-N(Rp)2 wherein each occurrence of Rp independently represents a C1 alkyl;
a C1-20alkylphenyl moiety optionally substituted with one or more —OR wherein R may represent H or C1-6alkyl;
a group of any one of the following formulae: 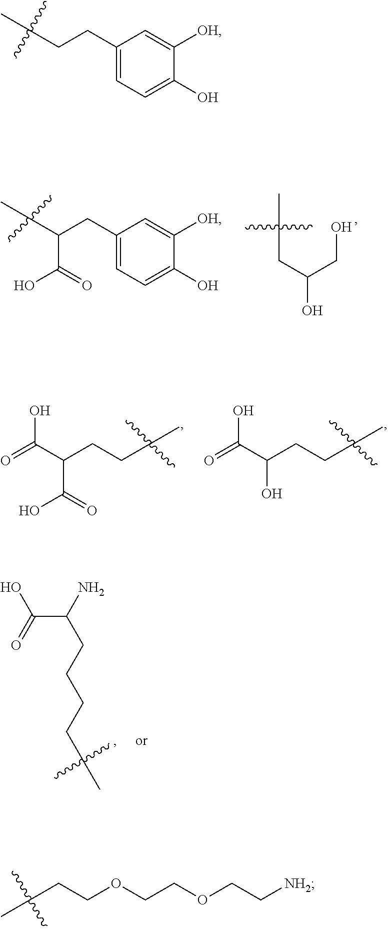
a hyaluronic acid, alginic acid, peptide, cellulose, amino acid, sugar (for example glucose, lactose or mannose derivatives) or oligonucleotide moiety; or
a C1-20alkyl or C1-20heteroalkyl moiety, preferably C1-6alkyl or C1-6heteroalkyl, most preferably C1-6alkyl, bearing an organosilica particle, preferably organosilica nanoparticles or core-shell nanocapsules, preferably the organosilica matrix may be porous, most preferably mesoporous, and may contain responsively cleavable bonds L₂ or responsively cleavable linkers #-R³-L₂-R⁴-# within the organosilica framework as defined in claim 6.

15. The method of claim 14, wherein the monomer precursor is of formula (IVa)

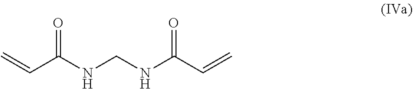

16. The method of claim 14 or 15, wherein the linker L₁ and *-R¹-L₁-R₂-* are as defined in claim 2.

17. The method of any one of claims 14 to 16 wherein the molecular crosslinker precursor A-R¹-L₁-R²-A is of formula

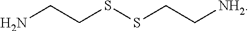

18. The method of any one of claims 14 to 17 wherein the selected precursor of formula B—R⁸ is of formula

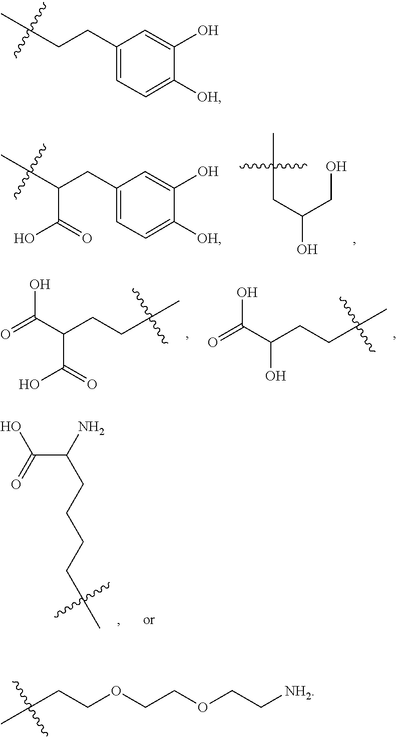

19. A hybrid hydrogel covalently non-covalently mixed with, or covalently conjugated to, organosilica nanoparticles or organosilica nanocapsules having a core/shell structure, obtainable by a method of any one of claims 14 to 18; wherein the organosilica matrix of the organosilica nanoparticles or core/shell nanocapsules may preferably be porous, most preferably mesoporous, and wherein the organosilica matrix of the nanoparticles or nanocapsules may be disintegrable and may contain responsively cleavable bridges #-R³-L₂-R⁴-# between Si atoms within the organosilica framework as defined in claim 6.

20. A hybrid hydrogel of any one of claims 1 to 12 or a pharmaceutical composition of claim 13, for use as medicament.

21. Hybrid hydrogel according to claim 20 for use in sealing a wound, for enhancing tissue regeneration, as fillers for example for submucosal fluid cushion for surgery, tissue reconstitution in a subject-in-need thereof, for the treatment of diabetes, for the treatment of spinal cord injury.

22. Hybrid hydrogel according to claim 20 for use as a medicament for the treatment of cancer, preferably tumor, more preferably for the resection of solid tumors.

23. A method for sealing acute and/or chronic wounds and/or perforation in a subject-in-need thereof, the method comprising administering to the subject a therapeutically effective amount of a hybrid hydrogel of any one of claims 1 to 12 or a pharmaceutical composition of claim 13, thereby sealing the wound and/or perforation.

24. A method for treating a disease, preferably cancer tumor, in a subject-in-need thereof, the method comprising administering to the subject a therapeutically effective amount of a hybrid hydrogel of any one of claims 1 to 12 or a pharmaceutical composition of claim 13, thereby treating the disease in the subject.

25. Use of a hybrid hydrogel of any one of claims 1 to 12, in a cosmetic composition.

26. Use of a hybrid hydrogel of any one of claims 1 to 12 or a cosmetic composition of claim 25, for delivering a cosmetically bioactive macromolecule to the skin.

27. Use according to claim 25 or 26, wherein the cosmetically bioactive macromolecule is collagen, keratin, elastin, calcitonin, hyaluronic acid, aminoacids, retinol, antioxidants, vitamins or silk proteins.

28. A method for systemically delivering a drug, or a bioactive macromolecule in a biologically active form, to a subject in need thereof, the method comprising, administering to the subject a therapeutically effective amount of a hybrid hydrogel of any one of claims 1 to 12 or a pharmaceutical composition of claim 13.

29. The method of claim 28, wherein said bioactive macromolecule is selected from proteins, oligonucleotides, antibodies, peptides, PNA, DNA, RNA, gene fragments, a hormone, a growth factor, a protease, an extra-cellular matrix protein, an enzyme, an infectious viral protein, an antisense oligonucleotide, a dsRNA, a ribozyme, a DNAzyme, antibiotics, antinflammatory, steroids, chemiotherapeutics.

30. A unit dosage form for local delivery of a molecule to a tissue of a subject, the unit dosage form comprising, a therapeutically effective amount of a hybrid hydrogel of any one of claims 1 to 12 or a pharmaceutical composition of claim 13, wherein said macromolecule is selected from proteins, oligonucleotides, antibodies, peptides, PNA, DNA, RNA, gene fragments, a hormone, a growth factor, a protease, an extra-cellular matrix protein, an enzyme, an infectious viral protein, an antisense oligonucleotide, a dsRNA, a ribozyme and a DNAzyme.

31. A delivery system for enhancing wound healing, tissue regeneration and/or tissue regeneration in vivo, said system comprising a hybrid hydrogel of any one of claims 1 to 12 or a pharmaceutical composition of claim 13.

32. A hybrid hydrogel according to any one of claims 1 to 12, for use in the treatment of fistula.

33. Hybrid hydrogel for use according to claim 32, wherein the hybrid hydrogel is non-covalently mixed with, or covalently conjugated to, organosilica nanoparticles or organosilica nanocapsules having a core/shell structure; wherein the organosilica matrix of the organosilica nanoparticles or core/shell nanocapsules may preferably be porous, most preferably mesoporous, and wherein the organosilica matrix of the nanoparticles or nanocapsules may be disintegrable and may contain responsively cleavable bridges #-R³-L₂-R⁴-# between Si atoms within the organosilica framework as defined in claim 6.

34. Hybrid hydrogel for use according to claims 32 or 33, in the treatment of acute or chronic fistula.

35. A method for treating fistula in a subject in need thereof, the method comprising administering to the subject a therapeutically effective amount of a hybrid hydrogel as defined any one of claims 1 to 12, or a composition according to claim 13 comprising a pharmaceutically acceptable carrier.

36. Method according to claim 35 for the treatment of acute or chronic fistula.
